(19) United States
(12) Patent Application Publication
(10) Pub. No.: US 2004/0005613 A1
(43) Pub. Date: Jan. 8, 2004

(54) Methods, probes, and accessory molecules for detecting single nucleotide polymorphisms

(76) Inventor: Michael L. Norton, Huntington, WV (US)

Correspondence Address: STEPTOE & JOHNSON PLLC, Bank One Center, Sixth Floor, P.O. Box 2190, Clarksburg, WV, 26302-2190, US

(21) Appl. No.: 10/441,495

(22) Filed: May 20, 2003

Related U.S. Application Data
(60) Provisional application No. 60/383,291, filed on May 22, 2002.
(60) Provisional application No. 60/387,831, filed on Jun. 10, 2002.

(51) Int. Cl. C12Q 1/68

(52) U.S. Cl. 435/6

(57) ABSTRACT

The present invention concerns the detection of single nucleotide polymorphisms in a sample. The present invention discloses methods for detecting single nucleotide polymorphisms in a sample. The present invention further discloses nucleic acid probes and accessory molecules useful in the methods of the invention.

The present application claims benefit of priority to the following applications, which are incorporated by reference in their entirety herein: U.S. Provisional Patent Application No. 60/383,291 to Norton, entitled “Method and apparatus for DNA sequence recognition and signaling”, filed on May 22, 2002, and U.S. Provisional Patent Application No. 60/387,831 to Norton, entitled “Method and apparatus for DNA sequence recognition and signaling”, filed on Jun. 10, 2002.

### TECHNICAL FIELD

The present invention relates generally to the field of molecular biology, and more specifically concerns methods, probes, and accessory molecules for detecting single nucleotide polymorphisms.

### BACKGROUND

Single nucleotide polymorphisms (SNPs) are nucleic acid sequence variations where a single nucleotide at a specific locus in a given nucleic acid sequence is changed, resulting in two or more allelic variants. A polymorphic locus is termed an SNP generally when the allele frequency of the most common allelic variant is less than 99%, that is, a given allelic variant must occur in at least 1% of the population. SNPs occur every few hundred nucleotides in the human genome and tend to be very stably inherited. Their prevalence and genetic stability make SNPs useful markers in genetic analysis. Applications include SNP analyses of specific sequence variations that are associated with a particular disease, and genetic screening of individual susceptibility to a disease associated with a particular SNP.

### BRIEF DESCRIPTION OF THE FIGURES

FIG. 1 depicts schematic, non-limiting examples of nucleic acid probes useful in the first and second methods of the present invention. Solid lines indicate structures that include a nucleic acid or nucleic acid mimic sequence or both. Dashed lines indicate structures that do not include a nucleic acid or nucleic acid mimic sequence or both. Legend: 1, first recognition sequence; 2, second recognition sequence; 3, linking element; star, first reporter moiety; and diamond, second reporter moiety. Drawings are not intended to be to scale. As shown in this figure, a nucleic acid probe may be linear (as in FIGS. 1A, 1B, 1C, and 1D) or non-linear (as in FIGS. 1E and 1F). The first recognition sequence need not be at a terminus of the nucleic acid probe. The second recognition sequence need not be at a terminus of the nucleic acid probe. The first reporter moiety may be located anywhere on the first recognition sequence, not necessarily on a terminus of the first recognition sequence. The second reporter moiety may be located anywhere on the second recognition sequence (as in FIGS. 1A, 1B, 1D, and 1E) or, alternatively, anywhere on the linking element (as in FIGS. 1C and 1F). The linking element may be attached, directly or by an intervening segment (which may be anywhere on the linking element, not necessarily at a terminus of the linking element), to a terminus of the first or second recognition sequences or to an internal location of the first or second recognition sequences. Nucleic acid probes useful in the third method of the present invention are similar, except that they do not include a second reporter moiety as part of their structure.

FIG. 2 depicts schematic, non-limiting examples of configurations of the first and second methods of the present invention, wherein the nucleic acid probe is hybridized to a target (FIGS. 2A, 2B, and 2G) in a two-stranded configuration, or wherein the nucleic acid probe is hybridized to a target and interacts (for example, by base-pairing) with an accessory molecule of the second method of the invention (FIGS. 2C, 2D, 2E, and 2F) in a three-stranded configuration. Solid lines indicate structures that include a nucleic acid or nucleic acid mimic sequence or both. Dashed lines indicate structures that do not include a nucleic acid or nucleic acid sequence or both. Arrowheads indicate directionality (either 5′ to 3′, or 3′ to 5′) of a nucleic acid or nucleic acid mimic sequence or both, and thus indicate the anti-parallel orientations of base-paired structures 1 and 4, 2 and 5, or 3 and 6. Legend: 1, first recognition sequence of the nucleic acid probe; 2, second recognition sequence of the nucleic acid probe; 3, linking element of the nucleic acid probe; star, first reporter moiety of the nucleic acid probe; diamond, second reporter moiety of the nucleic acid probe; 4, first site of a target (representing a first site of a target allelic variant of a single nucleotide polymorphism, containing the polymorphic locus); 5, second site of a target (representing a second site of a target allelic variant of a single nucleotide polymorphism); and 6, a portion of the accessory molecule that interacts (in this example, by base-pairing) with the linking element of the nucleic acid probe. Drawings are not intended to be to scale. Configurations of the third method of the present invention are similar, except that the second reporter moiety is located on the accessory molecule of the third method of the invention. FIG. 2A depicts a two-stranded configuration where the target sites 4 and 5 are contiguous, and the second reporter is located on the second recognition sequence 2. FIG. 2B depicts a two-stranded configuration where the target sites 4 and 5 are non-contiguous, and the second reporter is located on the linker element 3. FIG. 2C depicts a three-stranded configuration where the target sites 4 and 5 are non-contiguous, the second reporter is located on the linker element 3, and the accessory molecule is base-paired with the linker element. FIG. 2D depicts a three-stranded configuration where the target sites 4 and 5 are non-contiguous, the second reporter is located on the linker element 3, the accessory molecule is base-paired with the linker element, and the accessory molecule is attached to an internal location of the second recognition sequence. FIG. 2E depicts a three-stranded configuration where the target sites 4 and 5 are non-contiguous, the second reporter is located on the linker element 3, the accessory molecule is base-paired with the linker element, and the accessory molecule is attached to a terminus of the second recognition sequence. FIG. 2F depicts a three-stranded configuration where the target sites 4 and 5 are contiguous, the second reporter is located on the second recognition sequence 2, the accessory molecule is base-paired with the linker element, and the first recognition sequence, second recognition sequence, and accessory molecule form a continuous nucleic acid or nucleic acid mimic sequence. FIG. 2G depicts a two-stranded configuration where the target sites 4 and 5 are contiguous, and the second reporter is located on the second recognition sequence 2.

FIG. 3 depicts a double-crossover, antiparallel, even spacing (DAE) DNA nanoarray unit, the Block A unit, which consists of five strands of DNA as described in Example 1. The annealing processes of a self-assembling model system representing three of the five strands of Block A were examined using a nucleic acid probe, a target DNA strand, and an accessory molecule DNA strand.

FIG. 4 depicts the base-pairing between a nucleic acid probe (SEQ ID NO. 1), a target DNA strand (SEQ ID NO. 2), and an accessory molecule (SEQ ID NO. 3), as described in detail in Example 1. Base-paired sequences are indicated by the underlined nucleotides. The 3′ and 5′ termini of each strand are indicated by numbers. The nucleic acid probe is depicted in a circular arrangement, and the nucleotides to which the reporter moieties are attached indicated by bold letters. When fully hybridized, the nucleic acid probe is base-paired to both the target DNA strand and to the accessory molecule.

FIG. 5 depicts representative fluorescence spectra of the nucleic acid probe (SEQ ID NO. 1) alone (FIGS. 5A through 5E), or in combination with a target DNA strand (SEQ ID NO. 2) (FIGS. 5F through 5J), or in combination with an accessory molecule (SEQ ID NO. 3) (FIGS. 5K through 50). Fluorescence intensity is given in counts per second (cps). These spectra were obtained in the first set of experiments described in Example 1.

FIG. 6 depicts representative fluorescence spectra of the nucleic acid probe (SEQ ID NO. 1) alone (FIGS. 6A through 6E), or in combination with a target DNA strand (SEQ ID NO. 2) (FIGS. 6F through 6J, or in combination with an accessory molecule (SEQ ID NO. 3) (FIGS. 6K through 6O). Fluorescence intensity is given in counts per second (cps). These spectra were obtained in the second set of experiments described in Example 1.

FIG. 7 depicts temperature-dependent plots of the ratios of tetramethylrhodamine intensity to fluorescein intensity (FIG. 7A), the FRET efficiency (FIG. 7B), and the distance between the two fluorophores (FIG. 7C), calculated from fluorescence intensity values obtained in the first set of experiments described in Example 1.

FIG. 8 depicts temperature-dependent plots of the ratios of tetramethylrhodamine intensity to fluorescein intensity (FIG. 8A), the FRET efficiency (FIG. 8B), and the distance between the two fluorophores (FIG. 8C), calculated from fluorescence intensity values obtained in the second set of experiments described in Example 1.

FIG. 9 depicts a nucleic acid probe (SEQ ID NO. 1) and four target DNA strands representing different target allelic variants of a single nucleotide polymorphism (SNP), as used in experiments which demonstrated the sensitivity of the probe to a mismatch between the first recognition sequence of the nucleic acid probe and a first site of a target allelic variant of an SNP (see Example 3). The first target DNA strand.(SEQ ID NO. 10) represents an allelic variant of an SNP that perfectly complements the nucleic acid probe, with no base-pairing mismatches. The second, third, and fourth target DNA strands (SEQ ID NO. 11, SEQ ID NO. 12, and SEQ ID NO. 13) represent allelic variants of three SNPs (each with a polymorphic locus at a different site, as indicated by the arrow). Mismatched bases are indicated by italics. Base-paired sequences are indicated by the underlined nucleotides. The 3′ and 5′ termini of each strand are indicated by numbers. The nucleic acid probe is depicted in a circular arrangement, and the nucleotides to which the reporter moieties are attached indicated by bold letters.

FIG. 10 depicts representative fluorescence spectra of the nucleic acid probe (SEQ ID NO. 1) and the first target DNA strand (SEQ ID NO. 10) (FIG. 10A through 10E), where there is no single base-pairing mismatch, or of the nucleic acid probe (SEQ ID NO. 1 and the fourth target DNA strand (SEQ ID NO. 13) (FIGS. 10F through 10J), where there is a single base-pairing mismatch, as described in Example 3. Fluorescence intensity is given in counts per second (cps).

FIG. 11 depicts temperature-dependent plots of the ratios of tetramethylrhodamine intensity to fluorescein intensity, calculated from fluorescence intensity values of the nucleic acid probe (SEQ ID NO. 1) and the first, second, third, and fourth target DNA strands (SEQ ID NO. 10, SEQ ID NO. 11, SEQ ID NO. 12, and SEQ ID NO. 13), obtained in the experiments described in Example 3. In each of the three cases where there was a single base-pairing mismatch (nucleic acid probe and SEQ ID NO. 11, SEQ ID NO. 12, or SEQ ID NO. 13), a surprisingly large decrease in FRET efficiency was observed, relative to the case where there was no mismatch (nucleic acid probe and SEQ ID NO. 10).

FIG. 12 depicts a representative, high magnification AFM micrograph, as described in Example 4. Brighter portions of the image represent raised or elevated locations in the sample surface that are approximately 0.7 nanometers higher than the darkest features in the image. These bright image portions were attributed to individual nucleic acid probe molecules, bound to a single-stranded long RCA strand that is not visible in the image. Measurement lines overlaid on the image connect the centers of each bright image portion, and were estimated to have lengths, reading from left to right, of 38, 56, and 28 nanometers, respectively.

FIG. 13 depicts schematic, non-limiting examples of different systems employing methods and probes of the present invention as applied to various assay formats of two-stranded or three-stranded configurations, as described in Example 5. The heavy black line with a star and diamond represents a nucleic acid probe. The light line represents a target (such as a target DNA strand containing an SNP). The heavy grey line represents an accessory molecule. The dotted line and shaded rectangle represents a capture molecule attached to a solid substrate. Drawings are not intended to be to scale. FIG. 13A depicts a two-strand assay performed with all components in solution phase. The sample that may contain an SNP of interest is contacted with the nucleic acid probe. Under appropriate hybridization conditions, the nucleic acid probe hybridizes to the SNP and the resulting signal detected. FIG. 13B depicts a two-strand assay performed with one component on a solid substrate. The nucleic acid probe is affixed, via a capture molecule, to the surface of a solid substrate, and the SNP in solution is allowed to contact and hybridize to the nucleic acid probe. FIG. 13C depicts a three-strand assay performed with all components in solution phase. The nucleic acid probe is contacted with the accessory molecule, and the linking element of the nucleic acid probe base-pairs with a sequence of the accessory molecule. The resulting two-strand “capture device” (the nucleic acid probe/accessory molecule complex) is contacted with the sample containing an SNP of interest. Under appropriate hybridization conditions, the nucleic acid probe hybridizes to the SNP and the resulting signal detected. A suitable signal could also be generated in a parallel case where the first reporter moiety is located on the nucleic acid probe and the second reporter moiety is located on the accessory molecule. FIG. 13D depicts a three-strand assay performed on a solid substrate. A capture DNA strand is affixed to the surface of a solid substrate and binds and immobilizes the SNP. A complex including the nucleic acid probe hybridized to an accessory molecule is contacted with the SNP/capture DNA strand complex, and under appropriate hybridization conditions, the nucleic acid probe/accessory molecule complex hybridizes to the SNP and the resulting signal detected. FIG. 13E depicts an assay wherein multiple probes (of one type or of more than one type) on a single accessory molecule may be used to analyze a sample for one or more target allelic variants of an SNP of interest. Assays using the third method of the present invention are similar, except that the second reporter moiety is located on the accessory molecule of the third method of the invention.

FIG. 14 depicts a nucleic acid probe (SEQ ID NO. 20) and two target DNA strands representing the wild type allele (SEQ ID NO. 23) and the mutant allele (SEQ ID NO. 24) of the human hemochromatosis single nucleotide polymorphism, respectively, as described in Example 5. The nucleic acid probe (SEQ ID NO. 20) was designed to base-pair perfectly with the wild-type allele (SEQ ID NO. 23), and to base-pair with a single base-pairing mismatch with the mutant allele (SEQ ID NO. 24). Base-paired sequences are indicated by the underlined nucleotides. The 3′ and 5′ termini of each strand are indicated by numbers. The nucleic acid probe is depicted in a circular arrangement, and the nucleotides to which the reporter moieties are attached indicated by bold letters. Nucleotides at the polymorphic locus of the wild-type and mutant alleles are italicized and indicated by the arrow.

### SUMMARY

The present invention provides a first method for detecting a single nucleotide polymorphism in a sample, which includes the steps of contacting a sample with a nucleic acid probe including a first recognition sequence, a second recognition sequence, a linking element, and two reporter moieties, and allowing the probe and sample to hybridize, whereby the spatial arrangement of the two reporter moieties relative to each other changes and causes a change in a detectable signal that thus indicates the presence or absence of a single nucleotide mismatch between the probe and a target allelic variant of a single nucleotide polymorphism present in the sample. The first reporter moiety is located on the first recognition sequence of the nucleic acid probe, and the second reporter moiety may be located on the second recognition sequence of the nucleic acid probe or on the linking element of the nucleic acid probe. A change in the spatial arrangement of the first reporter moiety relative to the second reporter moiety results in a change in a detectable signal, whereby the relative change in detectable signal indicates the presence or absence of a single base-pairing mismatch between the nucleic acid probe and the target allelic variant of the single nucleotide polymorphism.

The present invention also provides a second method for detecting a single nucleotide polymorphism in a sample, which includes the steps of contacting a nucleic acid probe including a first recognition sequence, a second recognition sequence, a linking element, and two reporter moieties, with an accessory molecule and with a sample, and allowing the probe and sample to hybridize, whereby the spatial arrangement of the two reporter moieties relative to each other changes and causes a change in a detectable signal that thus indicates the presence or absence of a single nucleotide mismatch between the probe and a target allelic variant of a single nucleotide polymorphism present in the sample. The first reporter moiety is located on the first recognition sequence of the nucleic acid probe, and the second reporter moiety may be located on the second recognition sequence of the nucleic acid probe or on the linking element of the nucleic acid probe. A change in the spatial arrangement of the first reporter moiety relative to the second reporter moiety results in a change in a detectable signal, whereby the relative change in detectable signal indicates the presence or absence of a single base-pairing mismatch between the nucleic acid probe and the target allelic variant of the single nucleotide polymorphism.

The present invention further provides a third method for detecting a single nucleotide polymorphism in a sample, which includes the steps of contacting a nucleic acid probe including a first recognition sequence, a second recognition sequence, a linking element, and a reporter moieties, with an accessory molecule including a second reporter moiety and with a sample, and allowing the probe and sample to hybridize, whereby the spatial arrangement of the two reporter moieties relative to each other changes and causes a change in a detectable signal that thus indicates the presence or absence of a single nucleotide mismatch between the probe and a target allelic variant of a single nucleotide polymorphism present in the sample. The first reporter moiety is located on the first recognition sequence of the nucleic acid probe, and the second reporter moiety is located on the accessory molecule. A change in the spatial arrangement of the first reporter moiety relative to the second reporter moiety results in a change in a detectable signal, whereby the relative change in detectable signal indicates the presence or absence of a single base-pairing mismatch between the nucleic acid probe and the target allelic variant of the single nucleotide polymorphism.

Nucleic acid probes and accessory molecules for carrying out these methods are also provided. These probes and accessory molecules can include a deoxyribonucleic acid, a ribonucleic acid, a nucleic acid mimic, a peptide nucleic acid, a polypeptide, a polymer, or a combination thereof.

### DETAILED DESCRIPTION OF THE INVENTION

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. Conventional methods are used for these procedures, such as those provided in the art and various general references. Where a term is provided in the singular, the inventors also contemplate the plural of that term. The nomenclature used herein and the laboratory procedures described below are those well known and commonly employed in the art. Where there are discrepancies in terms and definitions used in references that are incorporated by reference, the terms used in this application shall have the definitions given herein. Other technical terms used herein have their ordinary meaning in the art that they are used, as exemplified by a variety of technical dictionaries (for example, Chambers Dictionary of Science and Technology, Peter M. B. Walker (editor), Chambers Harrap Publishers, Ltd., Edinburgh, UK, 1999, 1325 pp.). The inventors do not intend to be limited to a mechanism or mode of action. Reference thereto is provided for illustrative purposes only.

I. A First Method for Detecting a Single Nucleotide Polymorphism

The present invention provides a first method for detecting a single nucleotide polymorphism in a sample. The method can include the steps of: a) providing at least one sample suspected of containing a single nucleotide polymorphism; b) providing at least one nucleic acid probe, said at least one nucleic acid probe including: (i) a first recognition sequence that is complementary to a first site of a target allelic variant of said single nucleotide polymorphism, wherein said first site of a target allelic variant of said single nucleotide polymorphism includes a nucleotide at the polymorphic locus of said single nucleotide polymorphism; (ii) a second recognition sequence that is complementary to a second site of said target allelic variant of said single nucleotide polymorphism; (iii) a linking element that links said first and second recognition sequences, that is not complementary to either said recognition sequence; and (iv) a first reporter moiety, located on said first recognition sequence, and a second reporter moiety, wherein said first reporter moiety and said second reporter moiety are capable of interacting to produce a detectable signal; and a change in the spatial arrangement of said first reporter moiety relative to said second reporter moiety results in a change in said detectable signal; c) contacting said at least one sample with said at least one nucleic acid probe; d) incubating said at least one sample under hybridizing conditions with said at least one nucleic acid probe for a period of time sufficient to permit hybridization between said at least one nucleic acid probe and said target allelic variant of said single nucleotide polymorphism present in said at least one sample, wherein said hybridization changes said spatial arrangement of said first reporter moiety relative to said second reporter moiety; and relative said change in said spatial arrangement of said first reporter moiety relative to said second reporter moiety is different when there is a single nucleotide mismatch between said at least one nucleic acid probe and said target allelic variant of said single nucleotide polymorphism present in said at least one sample than when there is no single nucleotide mismatch; and e) detecting said change in said detectable signal, wherein relative said change in said detectable signal under said hybridization conditions is an indicator of the presence or absence of a single nucleotide mismatch between said at least one nucleic acid probe and said target allelic variant of said single nucleotide polymorphism present in said at least one sample. Preferably, the presence or absence of a given target allelic variant of said single nucleotide polymorphism is detected in the at least one sample.

Single Nucleotide Polymorphism

The single nucleotide polymorphism to be detected by a method of the invention can be any single nucleotide polymorphism (SNP) of interest. The term “single nucleotide polymorphism” encompasses any nucleic acid sequence, whether deoxyribonucleic acid (DNA) or ribonucleic acid (RNA), having a polymorphic, single-nucleotide locus, that is to say, any nucleic acid sequence variation where a single nucleotide at a specific locus (the “polymorphic locus”) in a given nucleic acid sequence may be any of at least two of the possible nucleotides (adenine, guanine, thymine, or cytosine for DNA; adenine, guanine, cytosine, or uracil for RNA) and thus gives rise to more than one allelic variant of that nucleic acid sequence. Although the term “single nucleotide polymorphism” is generally applied only to a naturally occurring nucleic acid sequence containing a polymorphic locus only when a given allelic variant of that nucleic acid sequence occurs in at least 1% of the population, the term as used herein encompasses not only such naturally occurring nucleic acid sequences that meet this limitation, but any naturally occurring or non-naturally occurring nucleic acid having a polymorphic, single-nucleotide locus, regardless of the occurrence rates of the allelic variants in a population. In some cases, the single nucleotide polymorphism to be detected by a method of the invention can be a non-nucleic acid analogue of an SNP, for example, a nucleic acid mimic SNP analogue, wherein the nucleic acid mimic SNP analogue includes a nucleic acid mimic sequence (such as a peptide nucleic acid sequence), having a polymorphic, single-base locus.

Sample

The sample to be subjected to a method of the present invention may be any sample of interest that is suspected of containing a single nucleotide polymorphism. The sample may include deoxynucleic acid or ribonucleic acid or both. The sample may be of entirely natural origin, of entirely non-natural origin (such as of synthetic origin), or a combination of natural and non-natural origins. A sample can be an environmental sample. A sample may include whole cells (such as prokaryotic cells, bacterial cells, eukaryotic cells, plant cells, fungal cells, or cells from multicellular organisms including invertebrates, vertebrates, mammals, and humans), tissues, organs, or biological fluids (such as, but not limited to, blood, serum, plasma, urine, semen, and cerebrospinal fluid). A sample may be an extract, containing a nucleic acid molecule, made from biological materials, such as from prokaryotes, bacteria, eukaryotes, plants, fungi, multicellular organisms or animals, invertebrates, vertebrates, mammals, non-human mammals, and humans. A sample may be an extract, containing a nucleic acid molecule, made from whole organisms or portions of organisms, cells, organs, tissues, fluids, whole cultures or portions of cultures, or environmental samples or portions thereof. A sample may include a plasmid, a cosmid, a fosmid, a phage, a bacterium, a virus, a bacterial artificial chromosome, a yeast artificial chromosome, or other nucleic acid vector. A sample may include a crude or semi-purified or purified nucleic acid or nucleic acid mimic preparation (for example, a phenol-chloroform-extracted nucleic acid, an ethanol-precipitated nucleic acid, a recombinant nucleic acid, a nucleic acid amplification reaction product, a nucleic acid transcription reaction product, a nucleic acid replication reaction product, a restriction fragment of a nucleic acid, a nucleic acid concentrated or purified by affinity chromatography or gel electrophoresis, or a synthetic peptide nucleic acid) such as may be prepared by methods known in the art (Molecular Cloning: A Laboratory Manual, Joseph Sambrook et al., Cold Spring Harbor Laboratory, 2001, 999 pp.; Short Protocols in Molecular Biology, Frederick M. Ausubel et al. (editors), John Wiley & Sons, 2002, 1548 pp.). A sample may be a product of an amplification reaction, such as, but not limited to, a polymerase chain reaction product, a reverse transcriptase amplification product, an antisense RNA amplification product (Phillips and Eberwine (1996) Methods, 10:283-288), a strand displacement amplification product (Walker et al. (1992), Nucleic Acids Res., 20:1691-1696), a Q-beta replicase-mediated amplification product (Lomeli et al. (1989) Clin. Chem., 35:1826-1831), a linked linear amplification product (Reyes et al. (2001) Clin. Chem., 47:31-40), a self-sustained sequence replication (3SR) product (Fahy et al. (1991) Genome Res., 1:25-33), or other nucleic acid amplification methods known in the art (Andras et al. (2001) Mol. Biotechnol., 19:29-44). A sample may include a nucleic acid located in situ within a cell or a tissue, such as, but not limited to, an in situ amplified nucleic acid (Long (1998) Eur. J Histochem., 42:101-109), or a chromosome, plasmid, or other cellular structure that contains a nucleic acid (Lichter et al. (1990), Science, 247:64-69). A sample may need minimal preparation (for example, collection into a suitable container) for use in a method of the present invention, or more extensive preparation (such as, but not limited to: removal, inactivation, or blocking of undesirable material, such as contaminants, undesired nucleic acids, or endogenous enzymes; filtration, size selection, or affinity purification; tissue or cell fixation, embedding, or sectioning; chromosome preparation and spreading; tissue permeabilization or cell lysis; methods to obtain nucleic acid molecule preparations such as nucleic acid amplification, concentration, or dilution; and preliminary denaturation of a nucleic acid sample).

Nucleic Acid Probe

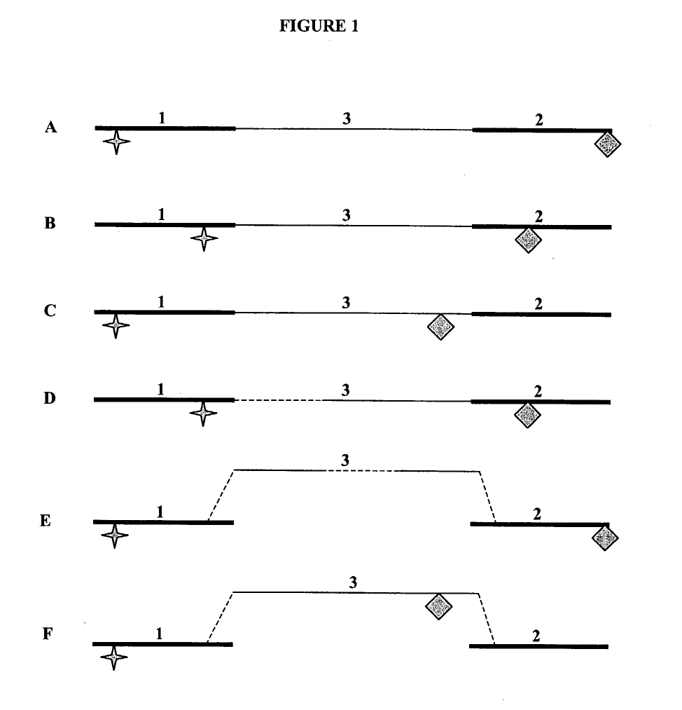

The nucleic acid probe used in the first method of the invention includes a first recognition sequence, a second recognition sequence, a linking element, and a first reporter moiety and a second reporter moiety. Representative, non-limiting nucleic acid probe designs are shown in FIG. 1. Preferably, the nucleic acid probe includes a deoxyribonucleic acid, a ribonucleic acid, a nucleic acid mimic (such as, but not limited to, a peptide nucleic acid), a polypeptide, a polymer, or a combination thereof. Most preferably, the nucleic acid probe includes a deoxyribonucleic acid, a ribonucleic acid, a nucleic acid mimic (such as, but not limited to, a peptide nucleic acid), or a combination thereof. Adjacent bases of the nucleic acid probe may be joined by a bond other than a phosphodiester bond (for example, adjacent modifided nucleotides or modified bases may be joined by an amide bond, a phosphonate bond, a phosphorothioate bond, phosphorodithionate bond, a phosphoroamidite bond, a phosphate ester bond, a siloxane bond, a carbonate bond, an ester bond, a thioester bond, an acetamide bond, a carbamate bond, an acrylamide bond, an ethyleneimine bond, an ether bond, a thioether bond, or a boron-containing bond such as a P-boranomethylphosphonate bond), as is known in the art (see, for example, Hamma and Miller (2003) Antisense Nucleic Acid Drug Dev., 13:19-30; Greenberg and Kahl (2001) J. Org. Chem., 66:7151-7154; Lin and Shaw (2001) Nucleosides Nucleotides Nucleic Acids, 20:1325-1328; Freier and Altmann (1997), Nucleic Acids Res., 25:4429-4443; Rice and Gao (1997) Biochemistry, 36:399-411; Agrawal et al. (1990), Proc. Natl. Acad. Sci. USA, 87:1401-1405; and Shabarova (1988), Biochimie, 70:1323-1334, which are herein incorporated in their entirety). Nucleic acid mimics are artificial molecules that are structurally and functionally analogous to naturally occurring nucleic acids (deoxyribonucleic acids and ribonucleic acids). Nucleic acid mimics used in the method of the invention include bases that are analogous to the nucleotides found in naturally occurring nucleic acids, and that are capable of complementary base pairing with the nucleotides in a naturally occurring nucleic acid. Non-limiting examples of a nucleic acid mimic include a nuclease-resistant boron-modified nucleotide polymer (Porter et al. (1997) Nucleic Acids Res., 25:1611-1617), and a peptide nucleic acid (PNA), which contains purine and pyrimidine bases, and which has an aminoethylglycine backbone in place of the sugar-phosphate backbone of a naturally occurring nucleic acid (Ganesh and Nielsen (2000) Curr. Org. Chem., 4:931-943; Ray and Norden (2000) FASEB J., 14:1041-1060; Egholm et al. (1992) J. Am. Chem Soc., 114:1895-1897).

The nucleic acid probe of the invention may be made by any technique suitable to the composition of the particular nucleic acid probe. For example, a nucleic acid probe may include only a nucleic acid (DNA or RNA) or only a nucleic acid mimic, and such a probe may be made by any suitable DNA, RNA, or nucleic acid mimic synthesis method. See, generally, Braasch and Corey (2001) Methods, 23:97-107; Hyrup and Nielsen (1996) Bioorg. Med. Chem., 4:5-23; Sprout (1993) Curr. Opin. Biotechnol., 4:20-28; and Gait (1991) Curr. Opin. Biotechnol., 2:61-68, which are herein incorporated in their entirety. The nucleic acid probe may be a hybrid or chimera, preferably including a nucleic acid (DNA or RNA or both) or a nucleic acid mimic (such as, but not limited to, a peptide nucleic acid) or both; the nucleic acid probe may further include a polypeptide, a polymer (such as polymeric plastics, silicones, fluorocarbons, polysaccharides, and the like), or a combination thereof. For example, a nucleic acid probe may include a first recognition sequence and a second recognition sequence composed of DNA, each connected by means of an intervening polypeptide segment to a linking element that includes a peptide nucleic acid, and first and second reporter moieties that make up a FRET pair. A nucleic acid probe that is such a hybrid or chimera may be manufactured by a combination of methods, including synthetic, semi-synthetic, enzymatic, recombinant, biological, or a combination thereof. See, generally, U.S. Pat. No. 6,204,326, issued Mar. 20, 2001, to Cook et al.; U.S. Pat. No. 5,539,083, issued Jul. 23, 1996, to Cook et al.; Tian and Wickstrom (2002) Org. Lett., 4:4013-4016; Niemeyer (2002) Trends Biotechnol., 20:395-401; Beier and Hoheisel (1999) Nucleic Acids Res., 27:1970-1977; Efimov et al. (1999) Nucleic Acids Res., 27:4416-4426; Koppitz et al. (1998) J. Am. Chem. Soc., 120:4563-4569; and Misra et al. (1998) Biochemistry, 37:1917-1925, which are herein incorporated in their entirety.

First Recognition Sequence of the Nucleic Acid Probe

The first recognition sequence of the nucleic acid probe is a sequence that is complementary to a first site of a target allelic variant of the single nucleotide polymorphism (SNP) of interest, and that includes a deoxyribonucleic acid, a ribonucleic acid, a nucleic acid mimic (such as, but not limited to, a peptide nucleic acid), or a combination thereof. The “first site of a target allelic variant” includes a nucleotide at the polymorphic locus of the SNP nucleic acid sequence, that is to say, a nucleotide at the specific locus in the given SNP nucleic acid sequence where the nucleotide may be any of at least two of the possible nucleotides (adenine, guanine, thymine, or cytosine for DNA; adenine, guanine, cytosine, or uracil for RNA), thus giving rise to more than one allelic variant of that nucleic acid sequence. Each base of the first recognition sequence of the nucleic acid probe is complementary to a nucleotide at a corresponding locus in the sequence of the first site of a target allelic variant of the SNP of interest. By “complementary” is meant that stable hydrogen bonding occurs between a purine base and a pyrimidine base according to Watson-Crick base-pairing rules, such as is seen in double-stranded naturally occurring nucleic acids where the pair of bases consists of a purine base (adenine or guanine) on one strand of nucleic acid and a pyrimidine base (thymine, cytosine, or uracil) on a second and opposite-running strand of nucleic acid. According to Watson-Crick base-pairing rules, adenine base-pairs with thymine (in deoxyribonucleic acids) or with uracil (in ribonucleic acids), and guanine base-pairs with cytosine. Analogous complementary base-pairing may also occur between bases of a nucleic acid mimic (such as, but not limited to, a peptide nucleic acid) and nucleotides of a naturally occurring nucleic acid. As a non-limiting example, if the sequence of the first site of a target allelic variant of the SNP of interest includes the 4 nucleotides ATCG (in the 5′ to 3′ direction), where the third nucleotide cytosine is located at the polymorphic locus and can occur as thymine in another allelic variant of the SNP of interest, then the first recognition sequence of the nucleic acid probe includes the 4 bases TAGC (in the 3′ to 5′ direction). The first recognition sequence of the nucleic acid probe may include any number of bases that permit the nucleic acid probe, under a given set of hybridization conditions, to differentially hybridize to any two particular allelic variants of an SNP, that is to say, that permit the first recognition sequence of the nucleic acid probe to base-pair more readily with one sequence of the first site of a target allelic variant of the SNP of interest than with a different sequence of the first site of the target allelic variant of the SNP, whereby the resulting detectable signals permit the particular allelic variants of an SNP to be distinguished from each other. In certain cases, the first recognition sequence of the nucleic acid probe may, under a given set of hybridization conditions, differentially hybridize to more than two particular allelic variants of an SNP, whereby the resulting detectable signals permit more than two particular allelic variants of an SNP to be separately distinguished from each other. In such situations, the nature of the mismatch, that is to say, the exact identity of the bases that form the mismatched pair, may influence the hybridization between the nucleic acid probe and the SNP.

Preferably, the first recognition sequence of the nucleic acid probe may include between about 4 and about 30 bases, or between about 4 and about 25 bases, or between about 4 and about 20 bases, or between about 4 and about 15 bases, or between about 4 and about 12 bases, or between about 4 and about 10 bases, or between about 4 and about 8 bases, or between about 4 and about 6 bases. However, the first recognition sequence of the nucleic acid probe may include any number of bases that allow the nucleic acid probe to differentially hybridize, resulting in a differential detectable signal upon hybridization, between any two target allelic variants of an SNP of interest, wherein there is no single base-pairing mismatch between the first recognition sequence and the first site of one target allelic variant of the SNP, and there is a single base-pairing mismatch between the first recognition sequence and the first site of the other target allelic variant of the SNP. The first recognition sequence of the nucleic acid probe need not be at a terminus of the nucleic acid probe. The exact sequence of any one first recognition sequence of a nucleic acid probe preferably takes into account the length of the first recognition sequence, the location and nature of the first reporter moiety, and the location of the mismatch in the sequence of the first site of a target allelic variant of the SNP of interest. For example, it is known that when an oligonucleotide probe of 8 bases binds to a DNA target, a mismatch located at either the 5′ or 3′ end of the probe is relatively less destabilizing than a mismatch located at an internal position, and when an oligonucleotide probe of 11 bases binds to a DNA target, a mismatch at a position 2 to 3 bases from either end of the probe is detectable (Fodor et al. (1993) Proceedings of the Robert A. Welch Foundation 37^{th} Conference on Chemical Research, 40 Years of the DNA Double Helix, 25-26 October 1993, Houston, Tex., USA, pp. 3-9, which is herein incorporated in its entirety). Generally, it is preferable to design a nucleic acid probe, specific for a target allelic variant of an SNP of interest, that, under a given set of hybridization conditions, is capable of hybridizing to the SNP of interest and producing a detectable signal that unambiguously or nearly unambiguously indicates the presence or absence of a single base mismatch between the nucleic acid probe and the target allelic variant of said single nucleotide polymorphism.

Second Recognition Sequence of the Nucleic Acid Probe

The second recognition sequence of the nucleic acid probe is a sequence that is complementary to a second site of a target allelic variant of the single nucleotide polymorphism (SNP) of interest, and that includes a deoxyribonucleic acid, a ribonucleic acid, a nucleic acid mimic (such as, but not limited to, a peptide nucleic acid), or a combination thereof. The “second site of a target allelic variant” includes a nucleotide sequence of the SNP of interest that does not include the polymorphic locus of the SNP. Each base of the second recognition sequence of the nucleic acid probe is complementary to a nucleotide at a corresponding locus in the sequence of the second site of a target allelic variant of the SNP of interest. As a non-limiting example, if the sequence of the second site of a target allelic variant of the SNP of interest includes the 4 nucleotides ATCG (in the 5′ to 3′ direction), where none of the nucleotides occurs at the polymorphic locus of the SNP, then the second recognition sequence of the nucleic acid probe includes the 4 bases TAGC (in the 3′ to 5′ direction). The first site of a target allelic variant of the SNP and the second site of the target allelic variant of the SNP may be a continuous nucleic acid sequence of the SNP, or, alternatively, may be a discontinuous nucleic acid sequence of the SNP wherein the first site of a target allelic variant of the SNP may be separated from the second site of the target allelic variant of the SNP by one or more bases. Preferably, the first site of a target allelic variant of the SNP does not include a sequence or sequences that are significantly complementary to a sequence or sequences of the second site of the target allelic variant of the SNP. Preferably, the first site of a target allelic variant of the SNP does not include an internal significantly complementary sequence or sequences, nor does the second site of the target allelic variant of the SNP include an internal significantly complementary sequence or sequences, wherein such an internal significantly complementary sequence allows an internal hairpin structure to form. Preferably, the second site of a target allelic variant of the SNP includes at least about 4 nucleotides. Preferably, the second recognition sequence of the nucleic acid probe includes at least 4 bases. Preferably, the second recognition sequence of the nucleic acid probe can include between about 4 and about 150 bases, or between about 4 and about 120 bases, or between about 4 and about 90 bases, or between about 4 and about 60 bases, or between about 4 and about 40 bases, or between about 4 and about 30 bases, or between about 4 and about 20 bases. However, the second recognition sequence of the nucleic acid probe may include any number of bases that permits the nucleic acid probe, when hybridized to the target allelic variant of the SNP of interest, to assume a configuration that permits the first reporter moiety to interact with the second reporter moiety and produce a detectable signal, such as is described in detail below. The second recognition sequence of the nucleic acid probe need not be at a terminus of the nucleic acid probe.

Linking Element of the Nucleic Acid Probe

The linking element of the nucleic acid probe is an element that links the first recognition sequence of the nucleic acid probe to the second recognition sequence of the nucleic acid probe. The linking element can include a deoxyribonucleic acid, a ribonucleic acid, a nucleic acid mimic (such as, but not limited to, a peptide nucleic acid), a polypeptide, a polymer, a combination thereof, or any moiety that serves to connect the two recognition sequences by covalent bonds or by non-covalent bonds. The linking element of the nucleic acid probe may be designed to be capable of complementary base-pairing with another nucleic acid sequence or nucleic acid mimic sequence (for example, a sequence of the accessory molecule of the second or third method of the invention, as described below). In such a case, the linking element of the nucleic acid probe can include at least one segment containing a deoxyribonucleic acid, a ribonucleic acid, a nucleic acid mimic (such as, but not limited to, a peptide nucleic acid), or a combination thereof, wherein the direction of any such-nucleic acid or nucleic acid mimic segment that is included in the linking element of the nucleic acid probe is anti-parallel to (that is, runs in the opposite direction from) the nucleic acid or nucleic acid mimic sequence that the linking element is intended to complement. Where the linking element of the nucleic acid probe is intended to base-pair with a nucleic acid sequence or nucleic acid mimic sequence of the accessory molecule, the linking element of the nucleic acid probe preferably includes at least one nucleic acid or nucleic acid mimic sequence of at least about 4 bases, or at least about 8 bases, or at least about 15 bases, and can include a sequence of up to about 60 bases, or up to about 120 bases, or up to about 300 bases. However, the linking element of the nucleic acid probe may include any number of bases that permits the nucleic acid probe, when hybridized to the target allelic variant of the SNP of interest, to assume a configuration that permits the first reporter moiety to interact with the second reporter moiety and produce a detectable signal, such as is described in detail below.

The linking element of the nucleic acid probe and the first recognition sequence of the nucleic acid probe may be a continuous sequence of the nucleic acid probe, or, alternatively, may be a discontinuous sequence of the nucleic acid probe wherein the linking element of the nucleic acid probe may be separated from the first recognition sequence of the nucleic acid probe by an intervening segment. The linking element may be attached, directly or by an intervening segment (which may be anywhere on the linking element), to a terminus of the first recognition sequence or to an internal location of the first recognition sequence. Where the linking element of the nucleic acid probe is separated from the first recognition sequence of the nucleic acid probe by an intervening segment, the intervening segment may include a deoxyribonucleic acid, a ribonucleic acid, a nucleic acid mimic (such as, but not limited to, a peptide nucleic acid), a polypeptide, a polymer, a combination thereof, or any moiety that serves to connect the two sequences by covalent bonds or by non-covalent bonds. The linking element of the nucleic acid probe and the second recognition sequence of the nucleic acid probe may be a continuous nucleic acid sequence of the nucleic acid probe, or, alternatively, may be a discontinuous nucleic acid sequence of the nucleic acid probe wherein the linking element of the nucleic acid probe may be separated from the second recognition sequence of the nucleic acid probe by an intervening segment. The linking element may be attached, directly or by an intervening segment (which may be anywhere on the linking element), to a terminus of the second recognition sequence or to an internal location of the second recognition sequence. Where the linking element of the nucleic acid probe is separated from the second recognition sequence of the nucleic acid probe by an intervening segment, the intervening segment may include a deoxyribonucleic acid, a ribonucleic acid, a nucleic acid mimic (such as, but not limited to, a peptide nucleic acid), a polypeptide, a polymer, a combination thereof, or any moiety that serves to connect the two sequences by covalent bonds or by non-covalent bonds. Preferably, the linking element of the nucleic acid probe does not include a sequence or sequences that are significantly complementary to a sequence or sequences of either or both of the first recognition sequence and the second recognition sequence of the nucleic acid probe. Preferably, the linking element of the nucleic acid probe does not include an internal significantly complementary sequence or sequences.

Reporter Moieties of the Nucleic Acid Probe

The nucleic acid probe includes a first reporter moiety, located on the first recognition sequence, and a second reporter moiety. The first reporter moiety and the second reporter moiety are capable of interacting to produce a detectable signal, which may be any signal that is convenient or desirable to detect. The examples of detectable signals that follow are not intended to be limiting. The detectable signal can arise from resonance energy transfer. For example, the first reporter moiety and the second reporter moiety may be two members of a resonance energy transfer pair, such as but not limited to a fluorescence resonance energy transfer (FRET) pair (for example, a pair of identical or different fluorophores), a luminescence resonance energy transfer (LRET) pair (for example, a luminescent lanthanide and an organic dye molecule) (Selvin & Hearst (1994), Proc. Natl. Acad. Sci. USA, 91:10024-10028), a bioluminescence resonance energy transfer (BRET) pair (for example, a bioluminescent protein and a fluorophore), or a phosphorescence resonance energy transfer (PRET) pair (for example, a phosphorescent compound and a fluorophore). The detectable signal may be a nuclear magnetic resonance (NMR) signal (for example, a nuclear Overhauser effect between a ¹⁹F-labelled first reporter moiety and a ¹⁹F-labelled second reporter moiety, or spin-spin coupling between a pair of nuclei that have different NMR chemical shifts), an electron spin resonance (ESR) signal or an electron paramagnetic resonance (EPR) signal (for example, the electron paramagnetic signal caused by spin-spin interaction of a pair of spin-labelled reporter moieties, such as a pair of spin-labelled nucleotides or a pair of nitroxide-labelled reporter moieties) (Rabenstein and Shin (1995) Proc. Natl. Acad. Sci. USA, 92:8239-8243), or an electromagnetic radiation signal (such as, but not limited to, wavelengths in the ultraviolet, visible, infrared, and X-ray spectrum). The detectable signal may be a change in the physical dimensions of the nucleic acid probe structure, such as a change in size or shape of the nucleic acid probe when hybridized to the target allelic variant of the SNP of interest, that may be detected by methods sensitive to physical dimensions, such as atomic force microscopy. The detectable signal may be produced by an enzymatic reaction, for example, where the first and second reporter moieties include an enzyme and its cofactor, or include fragments or subunits of an enzyme that must be close to each other for the enzyme to be active, or include an enzyme and its inhibitor.

The interaction between the first reporter moiety of the nucleic acid probe and the second reporter moiety of the nucleic acid probe may result in a detectable signal even when the probe is not hybridized to the target allelic variant of the SNP of interest. Alternatively, one or both of the reporter moieties of the nucleic acid probe may individually be capable of producing a detectable signal, preferably a detectable signal that is different from that produced by the interaction of the two reporter moieties, and most preferably a detectable signal that is different from that produced by the interaction of the two reporter moieties when the nucleic acid probe is hybridized to the target allelic variant of the SNP of interest. For example, where the two reporter moieties of the nucleic acid probe are two different fluorophores that make up a FRET pair, either or both of the fluorophores may be detected prior to hybridization. In this and analogous cases, it is thus possible to interrogate a system containing the nucleic acid probe and detect and optionally quantify the amount of the unhybridized probe present in the system, separately from detecting the signal produced by the interaction of the two reporter moieties when the nucleic acid probe is hybridized to the target allelic variant of the SNP of interest.

A change in the spatial arrangement of the first reporter moiety of the nucleic acid probe relative to the second reporter moiety of the nucleic acid probe preferably results in a change in the detectable signal produced by the interaction of the two reporter moieties. The change in spatial arrangement may be in terms of the distance between the two reporter moieties, such as where the distance between two members of a resonance energy transfer pair changes and a change in amount or efficiency of resonance energy transfer is observed, or where the distance between the two reporter moieties changes and a change in a nuclear Overhauser effect between the two moieties is observed. The change in spatial arrangement may be in terms of an angle, such as a change in the angle between a dipole moment of the first reporter moiety and a dipole moment of the second reporter moiety, or a change in dihedral angle formed by two bonds (observable as a change in coupling constants).

Preferably the detectable signal produced by the interaction of the two reporter moieties is a signal with an acceptable signal-to-noise ratio, that is to say, with a signal-to-noise ratio that is clearly distinguishable from background noise. The change in the detectable signal produced by the interaction of the two reporter moieties, and caused by a change in the spatial arrangement of the first reporter moiety of the nucleic acid probe relative to the second reporter moiety of the nucleic acid probe, may be an increase in the detectable signal, a decrease in the detectable signal, or a change in the nature of the detectable signal (for example, a change in ratios between fluorescent emissions of a FRET pair, a change in excited state lifetime in a time-resolved fluorescent spectrum, a change in coupling constants in a nuclear magnetic resonance spectrum, or a structural or configurational change that is detectable by methods sensitive to physical dimensions).

The first reporter moiety of the nucleic acid probe is located on the first recognition sequence of the nucleic acid probe. The first reporter moiety may be a reporter moiety covalently or non-covalently bonded to a base of, or elsewhere on, the first recognition sequence, or alternatively, a base of the first recognition sequence may itself include or make up the first reporter moiety. The first reporter moiety may be located on a terminal base of the first recognition sequence, or on an internal base of the first recognition sequence, or may be attached to the first recognition sequence by a spacer arm. In some cases, the first reporter moiety may interrupt the base sequence of the first recognition sequence (see, for example, Strässler et al. (1999) Helv. Chim. Acta, 82:2160-2171; Kool et al. (2002), Proceedings of the 23rd Army Science Conference, Dec. 2-5, 2002, Orlando, Fla., USA, Poster KP-01, “Use of Multiple Fluorescent Labels in the Detection of Biomolecules”). An example of a first reporter moiety covalently bonded to a base of, or elsewhere on, the first recognition sequence is a fluorophore covalently bonded to a nucleotide base, or alternatively, to a nucleotide phosphate group, of the first recognition sequence, where the first recognition sequence includes a nucleic acid sequence. A specific example of a first reporter moiety covalently bonded to a base of the first recognition sequence is fluorescein-dt, a modified thymine wherein fluorescein is attached to position 5 of the thymine ring by a six-carbon spacer arm, allowing insertion of a fluorescein-labelled, internal thymine of a nucleotide sequence (see, for example, www.idtdna.com/program/catalog/modifications.asp?catid=58, accessed May 1, 2003). A specific example of a first reporter moiety covalently bonded elsewhere on the first recognition sequence (in this case, to a nucleotide phosphate group) is tetramethylrhodamine attached by means of an N-hydroxysuccinimide functional group to a nucleotide phosphate modified with an amino-bearing crosslinking agent (see, for example, www.idtdna.com/program/catalog/modifications.asp?catid=58, accessed May 1, 2003). An example of a first reporter moiety non-covalently bonded to a base of the first recognition sequence is a fluorophore-labelled avidin non-covalently bonded to a biotinylated base of the first recognition sequence. An example of a base of the first recognition sequence that itself includes or makes up the first reporter moiety is a base of the first recognition sequence that is isotopically enriched in a magnetic nucleus (such as ¹⁵N, ¹³C, or ³¹ p), which may be detected by heteronuclear magnetic resonance spectroscopy (SantaLucia et al. (1995), Nucleic Acids Res., 23:4913-4921).

The second reporter moiety of the nucleic acid probe may be located on the second recognition sequence of the nucleic acid probe, or, alternatively, may on the linking element of the nucleic acid probe. The second reporter moiety may be located on a terminal base of the second recognition sequence, or on an internal base of the second recognition sequence, or may be attached to the second recognition sequence by a spacer arm. In some cases, the second reporter moiety may interrupt the base sequence of the second recognition sequence or of the linking element. Where the second reporter moiety is located on the second recognition sequence of the nucleic acid probe, the second reporter moiety may be covalently or non-covalently bonded to a base of, or elsewhere on, the second recognition sequence, or alternatively, a base of the second recognition sequence may itself include or make up the second reporter moiety. Where the second reporter moiety is located on the linking element of the nucleic acid probe, the second reporter moiety may be a reporter moiety covalently or non-covalently bonded to a base of, or elsewhere on, the linking element, or alternatively, a base of the linking element may itself include or make up the second reporter moiety.

The methods used to affix the reporter moieties to the nucleic acid probe depend on the nature of a given reporter moiety and the nature of the nucleic acid probe. Such methods include, for example, covalent cross-linking as well as non-covalent linking methods such as are known in the art (see, for example, R. P. Haugland, “Handbook of Fluorescent Probes and Research Products”, 9^{th} edition, J. Gregory (editor), Molecular Probes, Inc., Eugene, Oreg., USA, 2002, 966 pp.; Seitz and Kohler (2001), Chemistry, 7:3911-3925; and Pierce Technical Handbook, Pierce Biotechnology, Inc., 1994, Rockford, Ill.), isotopic enrichment (SantaLucia et al. (1995), Nucleic Acids Res., 23:4913-4921), or inclusion of a spin label (Bobst et al. (1984) J. Mol. Biol., 173:63-74) or a heavy atom (Irani and SantaLucia (1999) Tetrahedron Lett., 40:8961-8964). Where desired, for example when increased flexibility is needed, a reporter moiety may be affixed using a spacer arm (Keyes et al. (1997) Biophys. J., 72:282-90; Hustedt et al. (1995) Biochemistry, 34:4369-4375; and Pierce Technical Handbook, Pierce Biotechnology, Inc., 1994, Rockford, Ill.).

Where the second reporter moiety is located on the second recognition sequence of the nucleic acid probe, the first reporter moiety is located within about 15 bases, or within about 13 bases, or within about 10 bases, or within about 8 bases, or within about 6 bases, or within about 5 bases, or within about 4 bases, or within about 3 bases, from a terminus of the first recognition sequence of the nucleic acid probe (which need not be a terminus of the nucleic acid probe), and the second reporter moiety is located within about 75 bases, or within about 60 bases, or within about 45 bases, or within about 30 bases, or within about 20 bases, or within about 15 bases, or within about 10 bases, from a terminus of the second recognition sequence of the nucleic acid probe (which need not be a terminus of the nucleic acid probe). Where the second reporter moiety is located on the linking element of the nucleic acid probe, the first reporter moiety is located within about 15 bases, or within about 13 bases, or within about 10 bases, or within about 8 bases, or within about 6 bases, or within about 5 bases, or within about 4 bases, or within about 3 bases, from a terminus of the first recognition sequence of the nucleic acid probe (which need not be a terminus of the nucleic acid probe), and the second reporter moiety may be located anywhere on the linking element of the nucleic acid probe.

Contacting and Incubating

The first method of the present invention includes the steps of contacting and incubating at least one sample suspected of containing a single nucleotide polymorphism with at least one nucleic acid probe of the invention. By contacting is meant bringing the sample in fluid contact, preferably in liquid contact, with the nucleic acid probe. Where the sample includes the product of a reaction (such as, but not limited to, a nucleic acid amplification reaction, a nucleic acid transcription reaction, or a nucleic acid replication reaction), the nucleic acid probe may be contacted with the sample prior to, or after, the completion of the reaction; where the nucleic acid probe is designed to hybridize rapidly with the sample, the method of the invention may optionally serve to monitor in real-time the progress of the reaction.

The sample and the nucleic acid probe may both or either be in liquid solution (for example, in liquid aqueous solution), in liquid suspension (for example, in liquid aqueous suspension, colloidal suspension, or a suspension of liposomes, micelles, or lipid complexes), or attached, directly or indirectly, to a solid substrate (such as, but not limited to, the sides of a chamber such as a well, a cuvette, or a capillary, or the surface of chips, slides, films, membranes, meshes, gels, matrices, grids, beads, microbeads, magnetic beads, fibers, particulates, nanoparticles, conductors, semiconductors, or a microarray) or to a molecular structure (such as, but not limited to, dendrimers, polymers, polypeptides, proteins, glycoproteins, carbohydrates, nucleic acids, nucleic acid mimics, nucleic acid complexes, lipid films or membranes, ceramics, metals or metal oxides, or combinations thereof). Affixing the nucleic acid probe to a solid surface increases the effective concentration of the probe in the region close to the solid surface, and may confer additional advantages (such as reducing the amount of reagents needed, increasing the number of assays that can be performed in a given space, and defining a discrete location to be monitored for a detectable signal). Preferably, affixing the nucleic acid probe to a solid surface does not significantly interfere with the ability of the probe to hybridize with the sample. Most preferably, affixing the nucleic acid probe to a solid surface enhances the rate or efficiency of the hybridization. One example of contacting is dispensing by an automated liquid handling device a volume of aqueous solution that contains the sample into a microtiter plate well that contains the nucleic acid probe attached, directly or indirectly, to the sides of the well. Another example is dispensing by pipette a volume of sample onto discrete spots on a glass slide, wherein each spot contains a nucleic acid probe, specific for a particular allelic variant of one or more SNPs of interest, affixed to the surface of the slide. Another example is in situ intracellular delivery of a nucleic acid probe, for example, of a nucleic acid probe in a suspension of liposomes, micelles, or lipid complexes (Byk et al. (1998) J. Med. Chem., 41:229-235; Fraley et al. (1981) Biochemistry, 20:6978-6987), to a sample contained in a whole cell or intact tissue. Where the sample or the nucleic acid probe is attached, directly or indirectly, to a solid substrate or to a molecular structure, the attachment may be by covalent or by non-covalent means or by both, and may include a spacer moiety, such as a spacer arm. Covalent means are well-known in the art and may include, for example, the use of reactive groups, chemical modification or activation, photoactivated cross-linking, or bifunctional or trifunctional cross-linking agents (Pierce Technical Handbook, Pierce Biotechnology, Inc., 1994, Rockford, Ill.). Non-covalent means include but are not limited to physical adsorption, electrostatic forces, ionic interactions, hydrogen bonding, hydrophilic-hydrophobic interactions, van der Waals forces, and magnetic forces. The nucleic acid probes may in some instances be reusable, for example, when a previous sample is removed by washing or by heating.

The ratio of the nucleic acid probe to the target allelic variant of the SNP of interest need not be equal. Thus, one or more nucleic acid probes (differing in their first recognition sequences) may be, individually and separately, contacted and incubated with a sample that may contain one or more allelic variants of the SNP. Alternatively, one or more nucleic acid probes (differing in their first recognition sequence and in the detectable signal produced upon hybridization) may be severally contacted and incubated with a sample that may contain one or more allelic variants of the SNP.

The sample is incubated with the nucleic acid probe under hybridizing conditions for a period of time sufficient to permit hybridization between the nucleic acid probe and the target allelic variant of the single nucleotide polymorphism (SNP) of interest, if the SNP is present in the sample. By hybridization is meant complementary base-pairing between a sequence of bases on a first nucleic acid (or nucleic acid mimic) strand and a sequence of bases on a second nucleic acid (or nucleic acid mimic) strand. Preferably, the hybridized structure includes at least 4 consecutive base pairs. Preferably, hybridization conditions are selected to achieve significant hybridization between the nucleic acid probe and the target allelic variant of the single nucleotide polymorphism SNP of interest. Most preferably, hybridization conditions are selected to achieve quantitative or near-quantitative hybridization between the nucleic acid probe and the target allelic variant of the SNP of interest.

Hybridization is dependent on factors known in the art (see for example, Non-radioactive In Situ Hybridization Application Manual, Roche Applied Science, 2002, Indianapolis, Ind., pp. 33-37), including, but not limited to, the length and specific sequence of the base sequences between which complementary base-pairing occurs, the effective concentrations of the nucleic acid probe and the target allelic variant of the SNP of interest, the temperature of the hybridization mixture, the nature of the solvent, the amount of any components (for example, inorganic ions, especially monovalent or divalent cations, or organic solutes such as formamide or dextran sulfate, included in the solvent). Certain factors may be more easily or more conveniently controlled, such as the temperature or the ionic strength of the hybridization mixture. The melting temperature (Tₘ, the temperature at which half of the strands of a complementary pair of nucleic acid strands are unpaired) of a complementary pair of nucleic acid strands may be calculated (for reactions where the monovalent cation concentration is from between 0.01 to 0.20 moles per liter) by the “percent GC method”, given in Equation 1:

Tₘ=16.6 log M+0.41(GC)+81.5-0.72(F) (Equation 1)

where Tₘ is given in degrees Celsius, M is the monovalent cation concentration in moles per liter, GC is the molar percentage of guanine plus cytosine bases, and F is the percentage of formamide in the solution. Where the monovalent cation concentration is high (greater than 0.4 moles per liter), the component M may be deleted from Equation 1. This method is based on the fact that guanine and cytosine are more strongly hydrogen bonded, and thus more strongly base-paired, than are adenine and thymine. Mismatching of base pairs reduces both hybridization rates and thermal stability of the resulting duplexes. For large (containing more than 500 nucleotides) probes, for example, Tm decreases about 1 degree Celsius per percent base mismatch. However, these general rules may not extrapolate to hybridization with shorter sequences (less than 500 nucleotides) or oligonucleotides, which may be less predictable because of their small size (Nonradioactive In Situ Hybridization Application Manual, Roche Applied Science, 2002, Indianapolis, Ind., pp. 33-37).

An alternative method for estimating the annealing temperature (T_{d}, the temperature at which half of the strands of a complementary pair of nucleic acid strands are unpaired), applicable to hybridization of oligonucleotides of fewer than 50 base pairs (preferably of 14 to 20 base pairs), is the Wallace rule, given by Equation 2:

T_{d}=2(AT)+4(GC) (Equation 2)

where T_{d} is given in degrees Celsius, AT is the sum of the number of adenine and thymine bases present, and CG is the sum of the number of cytosine and guanine bases present. It is recommended that 8 degrees Celsius be added to the calculated value for oligonucleotides with more than 20 base pairs to convert T_{d} to Tₘ.

The period of time of incubation is preferably sufficient to permit significant hybridization between the nucleic acid probe and the target allelic variant of the single nucleotide polymorphism SNP of interest, and most preferably sufficient to permit quantitative or near-quantitative hybridization between the nucleic acid probe and the target allelic variant of the SNP of interest. The period of time also depends on the nature of the sample. For example, a sample that is highly purified and concentrated DNA in solution may require only a short hybridization time (such as from between about 1 second to about 1 minute or between about 1 second and about 10 minutes), whereas a sample that is a nucleic acid in situ in a cell or a tissue may require an extended hybridization time (such as from about 4 hours to overnight or about 24 hours). For convenience, the period of time is most preferably the shortest period of time that permits a amount of hybridization between the nucleic acid probe and the target allelic variant of the SNP of interest that is satisfactory for a specific purpose. The preferred concentration of the reactants (in particular, of the nucleic acid probe and the sample), is one that allows a detectable signal, under the hybridization conditions selected for that particular combination, that gives an acceptable signal-to-noise (that is to say, the amount of signal due to the specific assay response divided by the background signal) ratio for the particular instrument or means of detecting the signal. Preferably, the concentration of the reactants is also chosen to minimize costs.

Hybridization

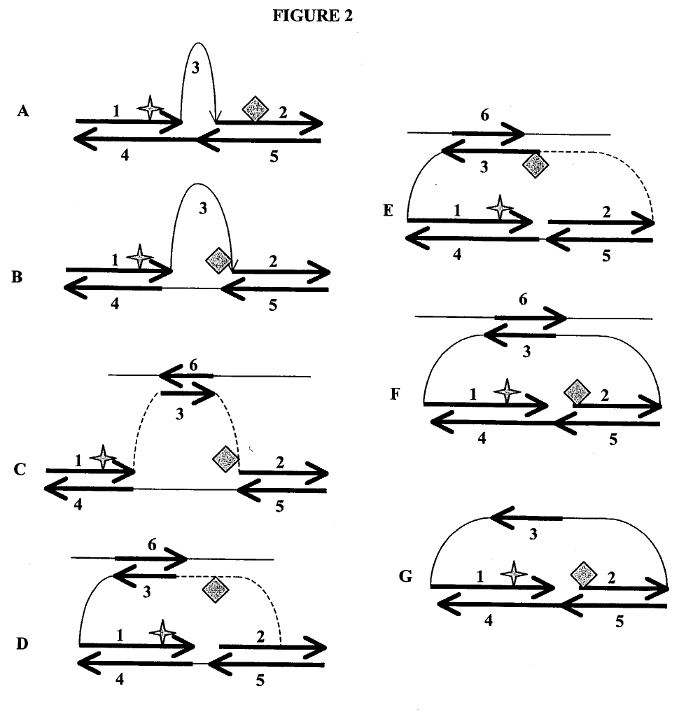

Under suitable hybridization conditions, the nucleic acid probe hybridizes with the target allelic variant of the single nucleotide polymorphism (SNP) of interest, if the SNP is present in the sample. When the nucleic acid probe is fully hybridized with the target allelic variant of the SNP, the first recognition sequence of the nucleic acid probe is hybridized to the first site of a target allelic variant of the SNP, and the second recognition sequence of the nucleic acid probe is hybridized to the second site of a target allelic variant of the SNP. This hybridization results in the nucleic acid probe and the target allelic variant of the SNP forming a configuration that may be described as a circular or looped structure, where the first and the second recognition sites of the nucleic acid probe are base-paired to the first and the second sites, respectively, of the target allelic variant of the SNP, and where the linking element of the nucleic acid probe is not base-paired to the target allelic variant of the SNP and thus forms the “open” portion of the circular or looped structure. Non-limiting examples of such circular or looped structures are shown in FIG. 2.

The direction (for example, whether 5′ to 3′, or 3′ to 5′, when referring to the hydroxyl groups at the 5′- and 3′ positions of the deoxyribose or ribose of a nucleic acid, of whether from amino to carboxyl, or carboxyl to amino, when referring to the modified glycine backbone of a peptide nucleic acid), of a nucleic acid or nucleic acid mimic portion of the nucleic acid probe need not be a single direction. Thus, a nucleic acid probe of the invention can have more than one or more segments including a nucleic acid or nucleic acid mimic, wherein each segment can run in the same (parallel) or different (anti-parallel) direction as another segment. The first and second recognition sequences and the linking element of the nucleic acid probe may include such segments which can run parallel or anti-parallel to each other. The direction of any such nucleic acid or nucleic acid mimic segment that is included in the first recognition sequence is anti-parallel to (that is, runs in the opposite direction from) the nucleic acid or nucleic acid mimic sequence of the first site of the target allelic variant of the SNP. The direction of any such nucleic acid or nucleic acid mimic segment that is included in the second recognition sequence is anti-parallel to (that is, runs in the opposite direction from) the nucleic acid or nucleic acid mimic sequence of the second site of the target allelic variant of the SNP.

When the nucleic acid probe is hybridized with the target allelic variant of the SNP, this hybridization preferably results in a change in the spatial arrangement of the first reporter moiety relative to the second reporter moiety, and thus changing the detectable signal that is a result of the interaction of the two reporter moieties. For example, where the first reporter moiety and the second reporter moiety are members of a fluorescence resonance energy transfer (FRET) pair, this hybridization may result in the two reporter moieties being brought within a distance sufficiently small to allow FRET to occur and to be detected. In another example, where the first reporter moiety and the second reporter moiety are, respectively, a base of the first reporter sequence and a base of the second reporter sequence that are isotopically enriched in a magnetic nucleus (such as ¹⁵N, ¹³C, or ³¹P), this hybridization may change the spatial arrangement (in terms of through-space distance or in terms of angle) of the isotopically enriched magnetic nuclei contained in these bases, resulting in a detectable change in the magnetic nuclei's NMR spectra. In yet another example, where the first reporter moiety is a labelled or unlabelled base of the first recognition sequence and the second reporter moiety is a labelled or unlabelled base of the second reporter sequence, this hybridization may result in a structural or configurational change that is detectable by methods sensitive to physical dimensions, such as by atomic force microscopy. More preferably, under a given set of hybridization conditions, the relative change in the spatial arrangement of the first reporter moiety relative to the second reporter moiety, and thus, the relative change in the detectable signal that is a result of the interaction of the two reporter moieties, is different when there is a single base-pairing mismatch between the nucleic acid probe and the target allelic variant of the SNP, than when there is no single base-pairing mismatch between the nucleic acid probe and the target allelic variant of the SNP. Most preferably, under a given set of hybridization conditions, the relative change in the spatial arrangement of the first reporter moiety relative to the second reporter moiety, and thus, the relative change in the detectable signal that is a result of the interaction of the two reporter moieties, is different when there is a single base-pairing mismatch between the first recognition sequence of the nucleic acid probe and the first site of a target allelic variant of the SNP, than when there is no single base-pairing mismatch between the first recognition sequence of the nucleic acid probe and the first site of a target allelic variant of the SNP. For example, where the first reporter moiety and the second reporter moiety are members of a fluorescence resonance energy transfer (FRET) pair that produce a detectable FRET signal only when the nucleic acid probe and target allelic variant of the SNP are significantly hybridized, and where, under a given set of hybridization conditions, the first recognition sequence hybridizes to the first site of a target allelic variant of the SNP only when there is no single base-pairing mismatch between the first recognition sequence and the first site of a target allelic variant of the SNP, then the appearance of a detectable FRET signal under a given set of hybridization conditions is an indicator of the absence of a single base-pairing mismatch between the first recognition sequence and the first site of a target allelic variant of the SNP.

Detecting

The detectable signal produced by the interaction between the first reporter moiety and the second reporter moiety may be detected by any means suitable to the type of signal produced. Suitable means include spectrophotometers, fluorimeters, luminometers, nuclear magnetic resonance (NMR) spectrometers, electron spin resonance (ESR) spectrometers, electron paramagnetic resonance (EPR) spectrometers, cameras, charge-coupled detectors, photodiodes, photodiode arrays, photomultipliers, or other light sensors with filters or wavelength selection filters or devices, light microscopes, fluorescence microscopes, epifluorescence microscopes, confocal microscopes, electron microscopes, near field scanning optical microscopes, far field confocal microscopes, scanning probe microscopes (such as scanning tunneling microscopes and atomic force microscopes), or a combination of these. Where the detection means requires excitation of one or both of the reporter moieties, excitation may be more or less specific (for example, excitation of a fluorophore by a narrow wavelength range or by a broader wavelength range). In some instances, the detection means may be capable of detecting a single unit or single molecule of a nucleic acid probe (Böhmer and Enderlein (2003), J. Opt. Soc. Am. B. 20:554-559; Single Molecule Detection in Solution: Methods and Applications, C. Zander, J. Enderlein, and R. A. Keller (editors), Wiley-VCH, Berlin and New York, 2002; Böhmer et al. (2002), Chem. Phys. Lett., 353:439-445; Nie and Zare (1997), Ann. Rev. Biophys. Biomol. Struct., 26:567-596). The detection means may be adapted to detect a signal in different assay formats, for example, single-use chambers (such as tubes or cuvettes), flow-through chambers, microtiter plates, microarrays, spots on a hybridization slide or chip, beads, optical fibers, and the like. The detection means may form part of a larger apparatus (which may be suited to high-throughput screening), such as a microplate reader, a liquid chromatograph, an electrophoretic capillary apparatus, a sheath-flow apparatus (such as a flow cytometer), or a video apparatus.

II. A Second Method for Detecting a Single Nucleotide Polymorphism

The present invention provides a second method for detecting a single nucleotide polymorphism in a sample. The method can include the steps of: a) providing at least one sample suspected of containing a single nucleotide polymorphism; b) providing at least one nucleic acid probe, said at least one nucleic acid probe including: (i) a first recognition sequence that is complementary to a first site of a target allelic variant of said single nucleotide polymorphism, wherein said first site of a target allelic variant of said single nucleotide polymorphism includes a nucleotide at the polymorphic locus of said single nucleotide polymorphism; (ii) a second recognition sequence that is complementary to a second site of said target allelic variant of said single nucleotide polymorphism; (iii) a linking element that links said first and second recognition sequences, that is not complementary to either said recognition sequence; and (iv) a first reporter moiety, located on said first recognition sequence, and a second reporter moiety, wherein said first reporter moiety and said second reporter moiety are capable of interacting to produce a detectable signal, and a change in the spatial arrangement of said first reporter moiety relative to said second reporter moiety results in a change in said detectable signal; c) providing at least one accessory molecule; d) contacting said at least one nucleic acid probe with said at least one accessory molecule; e) contacting said at least one nucleic acid probe and said at least one accessory molecule with said at least one sample; f) incubating said at least one sample under hybridizing conditions with said at least one nucleic acid probe and said at least one accessory molecule for a period of time sufficient to permit hybridization between said at least one nucleic acid probe and said target allelic variant of said single nucleotide polymorphism present in said at least one sample, wherein said hybridization changes said spatial arrangement of said first reporter moiety relative to said second reporter moiety; and relative said change in said spatial arrangement of said first reporter moiety relative to said second reporter moiety is different when there is a single base-pairing mismatch between said at least one nucleic acid probe and said target allelic variant of said single nucleotide polymorphism present in said at least one sample than when there is no single base-pairing mismatch; and g) detecting said change in said detectable signal, wherein relative said change in said detectable signal under said hybridization conditions is an indicator of the presence or absence of a single base-pairing mismatch between said at least one nucleic acid probe and said target allelic variant of said single nucleotide polymorphism present in said at least one sample. Preferably, the presence or absence of a given target allelic variant of said single nucleotide polymorphism is detected in the at least one sample.

The second method for detecting a single nucleotide polymorphism (SNP) in a sample is similar to the first method as described above under “A first method for detecting a single nucleotide polymorphism”. More specifically, the single nucleotide polymorphism, sample, nucleic acid probe and its component first and second recognition sequences and linking element, the first and second reporter moieties (and the detectable signal produced by their interaction), and detecting steps are generally as described above under “A first method for detecting a single nucleotide polymorphism”. The second method differs from the first primarily in that the second method includes the additional step of providing an accessory molecule, which is contacted and incubated with the nucleic acid probe and sample. This and associated differences are more fully described as follows.

Accessory Molecule

The accessory molecule useful in the second method of the invention may include a deoxyribonucleic acid, a ribonucleic acid, a nucleic acid mimic (such as, but not limited to, a peptide nucleic acid), a polypeptide, a polymer, or a combination thereof. Nucleic acid mimics are artificial molecules that are structurally and functionally analogous to naturally occurring nucleic acids (deoxyribonucleic acids and ribonucleic acids). Nucleic acid mimics used in the method of the invention include bases that are analogous to the nucleotides found in naturally occurring nucleic acids, and that are capable of complementary base pairing with the nucleotides in a naturally occurring nucleic acid. A non-limiting example of a nucleic acid mimic is a peptide nucleic acid (PNA), which contains purine and pyrimidine bases, and which has an aminoethylglycine backbone in place of the sugar-phosphate backbone of a naturally occurring nucleic acid. The accessory molecule can be of any size or length suitable to a particular application. The accessory molecule can be linear or branched (including multiply branched) or circular.

The accessory molecule of the second method of the invention may be made by any technique suitable to the composition of the particular accessory molecule, as described above under the subheading “Nucleic acid probe” under the heading “A first method for detecting a single nucleotide polymorphism”. For example, an accessory molecule may include only a nucleic acid (DNA or RNA) or only a nucleic acid mimic, and such an accessory molecule may be made by any suitable DNA, RNA, or nucleic acid mimic synthesis method. The accessory molecule may be a hybrid or chimera, preferably including a nucleic acid (DNA or RNA or both) or a nucleic acid mimic (such as, but not limited to, a peptide nucleic acid) or both; the accessory molecule may further include a polypeptide, a polymer (such as polymeric plastics, silicones, fluorocarbons, polysaccharides, and the like), or a combination thereof. An accessory molecule that is such a hybrid or chimera may be manufactured by a combination of methods, including synthetic, semi-synthetic, enzymatic, recombinant, biological, or a combination thereof.

The mode of interaction between the accessory molecule and the nucleic acid probe is determined by the physical composition of the accessory molecule and the nucleic acid probe. For example, a nucleic acid sequence or nucleic acid mimic sequence of the accessory molecule may be capable of complementary base-pairing with a nucleic acid sequence or nucleic acid mimic sequence of the linking element of the nucleic acid probe; in such a case, the direction of any such nucleic acid or nucleic acid mimic segment that is included in the accessory molecule is anti-parallel to (that is, runs in the opposite direction from) the nucleic acid or nucleic acid mimic sequence of the linking element of the nucleic acid probe. Where the accessory molecule is intended to complementary base-pair with a nucleic acid sequence or nucleic acid mimic sequence of the linking element of the nucleic acid probe, the accessory molecule preferably includes a nucleic acid or nucleic acid mimic sequence of at least about 4 bases, or at least about 8 bases, or at least about 15 bases, and can include a sequence of up to about 60 bases, or up to about 120 bases, or up to about 300 bases. In another example, the accessory molecule may include a polypeptide (such as a zinc-binding polypeptide domain) that is capable of binding the nucleic acid probe. In another example, an accessory molecule may be labelled with avidin and thus may bind a nucleic acid probe that is labelled with biotin. In another example, the accessory molecule may first optionally bind to the linking element of the nucleic acid probe by complementary base-pairing, followed by photo-activated cross-linking of the accessory molecule to the nucleic acid probe. In other words, the accessory molecule may associate with the nucleic acid probe by any suitable interaction or interactions, covalent or non-covalent, not limited solely to base-pairing, that permit the accessory molecule to function as intended.

The accessory molecule can serve one or more functions. One function may be where the accessory molecule helps to maintain a spatial arrangement (in terms of distance or angle) between the first reporter moiety and the second reporter moiety of the nucleic acid probe that is different when the nucleic acid probe is hybridized to the SNP than when the nucleic acid probe is not hybridized to the SNP. For example, in the case where the two reporter moieties are members of a FRET pair located on the nucleic acid probe, and where the linking element of the nucleic acid probe can complementary base-pair with a sequence of the accessory molecule, the accessory molecule, when hybridized to the nucleic acid probe, can maintain the two reporter moieties at a distance large enough to prevent significant intramolecular FRET from occurring, and thus minimizing false positive signals thus caused. Another function may be where the accessory molecule enhances the hybridization between the nucleic acid probe and the target allelic variant of the SNP of interest present in the sample. For example, the accessory molecule may limit the range of internal motions of the nucleic acid probe (thus improving or enhancing the nucleic acid probe's ability to hybridize correctly to target allelic variant of the SNP), or limit the range of locations on the intended target (for example, a strand of DNA that contains the target allelic variant of the SNP) with which the nucleic acid probe can interact, thus improving or enhancing the stringency of the hybridization. Another function may be where the accessory molecule serves to tether the nucleic acid probe to a solid surface or to a molecular structure. For example, the accessory molecule can bind the nucleic acid probe (and thus the SNP, when the SNP is hybridized to the nucleic acid probe), to the surface of microbeads, magnetic particles, a microarray, or the surfaces of a chamber.

The second method of the invention includes the step of contacting at least one nucleic acid probe with at least one accessory molecule. The nucleic acid probe and accessory molecule may both or either be in liquid solution (for example, in liquid aqueous solution), in liquid suspension (for example, in liquid aqueous suspension, colloidal suspension, or a suspension of liposomes, micelles, or lipid complexes), or attached, directly or indirectly, to a solid substrate (such as, but not limited to, the sides of a chamber such as a well, a cuvette, or a capillary, or the surface of chips, slides, films, membranes, meshes, gels, matrices, grids, beads, microbeads, magnetic beads, fibers, particulates, nanoparticles, conductors, semiconductors, or a microarray) or to a molecular structure (such as, but not limited to, dendrimers, polymers, polypeptides, proteins, glycoproteins, carbohydrates, nucleic acids, nucleic acid mimics, nucleic acid complexes, lipid films or membranes, ceramics, metals or metal oxides, or combinations thereof). Affixing the accessory molecule to a solid surface increases the effective concentration of the accessory molecule in the region close to the solid surface, and may confer additional advantages (such as reducing the amount of reagents needed, increasing the number of assays that can be performed in a given space, and defining a discrete location to be monitored for a detectable signal). Preferably, affixing the accessory molecule to a solid surface does not significantly interfere with the ability of the accessory molecule to interact with the probe or with the sample. Most preferably, affixing the accessory molecule to a solid surface enhances the rate or efficiency of the hybridization.

The ratio of the accessory molecule to the nucleic acid probe need not be equal. A single accessory molecule may be used with a single nucleic acid probe, or with more than one nucleic acid probe. One example is a single accessory molecule that includes a multiplicity of repeating subunits (for example, along the length of a linear accessory molecule or located on branches of a branched or multiply branched accessory molecule), each of which associates with a single unit of a nucleic acid probe. Such a construct would permit a single accessory molecule to, for example, tether several units of a nucleic acid probe to a solid surface or a molecular structure, thus increasing the effective concentration of the nucleic acid probe at that discrete location. Where more than one nucleic acid probe is used with a single accessory molecule, the nucleic acid probes may be more than one unit of a single type of nucleic acid probe, or may be different types of nucleic acid probes. The accessory molecule, or the accessory molecule complexed with one or more nucleic acid probes, may in some instances be reusable, for example, when a previous sample is removed by washing or by heating.

The second method of the invention includes the step of contacting said at least one nucleic acid probe and said at least one accessory molecule with said at least one sample. As in the first method, the ratio of the nucleic acid probe to the target allelic variant of the SNP of interest need not be equal. Thus, one or more nucleic acid probes (associated with at least one accessory molecule and differing in their first recognition sequences) may be, individually and separately, contacted and incubated with a sample that may contain one or more allelic variants of the SNP. Alternatively, one or more nucleic acid probes (associated with at least one accessory molecule and differing in their first recognition sequence and in the detectable signal produced upon hybridization) may be severally contacted and incubated with a sample that may contain one or more allelic variants of the SNP.

The second method of the invention includes the step of incubating said at least one sample under hybridizing conditions with said at least one nucleic acid probe and said at least one accessory molecule for a period of time sufficient to permit hybridization between said at least one nucleic acid probe and said target allelic variant of said single nucleotide polymorphism present in said at least one sample. When the nucleic acid probe is fully hybridized with the target allelic variant of the SNP, this hybridization preferably results in a change in the spatial arrangement of the first reporter moiety relative to the second reporter moiety, and thus changing the detectable signal that is a result of the interaction of the two reporter moieties. More preferably, under a given set of hybridization conditions, the relative change in the spatial arrangement of the first reporter moiety relative to the second reporter moiety, and thus, the relative change in the detectable signal that is a result of the interaction of the two reporter moieties, is different when there is a single base-pairing mismatch between the nucleic acid probe and the target allelic variant of the SNP, than when there is no single base-pairing mismatch between the nucleic acid probe and the target allelic variant of the SNP. Most preferably, under a given set of hybridization conditions, the relative change in the spatial arrangement of the first reporter moiety relative to the second reporter moiety, and thus, the relative change in the detectable signal that is a result of the interaction of the two reporter moieties, is different when there is a single base-pairing mismatch between the first recognition sequence of the nucleic acid probe and the first site of a target allelic variant of the SNP, than when there is no single base-pairing mismatch between the first recognition sequence of the nucleic acid probe and the first site of a target allelic variant of the SNP.

III. A Third Method for Detecting a Single Nucleotide Polymorphism

The present invention provides a third method for detecting a single nucleotide polymorphism in a sample. The method can include the steps of: a) providing at least one sample suspected of containing a single nucleotide polymorphism; b) providing at least one nucleic acid probe, said at least one nucleic acid probe including: (i) a first recognition sequence that is complementary to a first site of a target allelic variant of said single nucleotide polymorphism, wherein said first site of a target allelic variant of said single nucleotide polymorphism includes a nucleotide at the polymorphic locus of said single nucleotide polymorphism; (ii) a second recognition sequence that is complementary to a second site of said target allelic variant of said single nucleotide polymorphism; (iii) a linking element that links said first and second recognition sequences, that is not complementary to either said recognition sequence; and (iv) a first reporter moiety, located on said first recognition sequence; c) providing at least one accessory molecule, said at least one accessory molecule including a second reporter moiety, wherein said first reporter moiety and said second reporter moiety are capable of interacting to produce a detectable signal; and a change in the spatial arrangement of said first reporter moiety relative to said second reporter moiety results in a change in said detectable signal; d) contacting said at least one nucleic acid probe with said at least one accessory molecule; e) contacting said at least one nucleic acid probe and said at least one accessory molecule with said at least one sample; f) incubating said at least one sample under hybridizing conditions with said at least one nucleic acid probe and said at least one accessory molecule for a period of time sufficient to permit hybridization between said at least one nucleic acid probe and said target allelic variant of said single nucleotide polymorphism present in said at least one sample, wherein said hybridization changes said spatial arrangement of said first reporter moiety relative to said second reporter moiety; and relative said change in said spatial arrangement of said first reporter moiety relative to said second reporter moiety is different when there is a single base-pairing mismatch between said at least one nucleic acid probe and said target allelic variant of said single nucleotide polymorphism present in said at least one sample than when there is no single base-pairing mismatch; and g) detecting said change in said detectable signal, wherein relative said change in said detectable signal under said hybridization conditions is an indicator of the presence or absence of a single base-pairing mismatch between said at least one nucleic acid probe and said target allelic variant of said single nucleotide polymorphism present in said at least one sample.

The third method for detecting a single nucleotide polymorphism (SNP) in a sample is similar to the second method as described above under “A second method for detecting a single nucleotide polymorphism”. More specifically, the single nucleotide polymorphism, sample, nucleic acid probe and its component first and second recognition sequences and linking element, the detectable signal produced by the interaction between the first and second reporter moieties, and the contacting, incubating, and detecting steps are generally as described above under “A second method for detecting a single nucleotide polymorphism”. The third method differs from the first primarily in that in the third method, the first reporter moiety is located on the first recognition sequence of the nucleic acid probe (as in the first and second methods for detecting an SNP), and the second reporter moiety is located not on the nucleic acid probe but on the accessory molecule. This and associated differences are more fully described as follows.

Accessory Molecule

The accessory molecule useful in the third method of the present invention is structurally similar to the accessory molucule of the second method of the invention, as described above under the subheading “Accessory molecule”, under the heading “A second method for detecting a single nucleotide polymorphism”; however, the accessory molecule of the third method of the invention further includes a second reporter moiety that is capable of interacting with the first reporter moiety (located on the nucleic acid probe) to produce a detectable signal, which may be any signal that is convenient or desirable to detect. The accessory molecule useful in the third method of the invention may include a deoxyribonucleic acid, a ribonucleic acid, a nucleic acid mimic (such as, but not limited to, a peptide nucleic acid), a polypeptide, a polymer, or a combination thereof. The accessory molecule can be of any size or length suitable to a particular application, can be linear or branched (including multiply branched) or circular, and may associate with the nucleic acid probe by any suitable interaction or interactions, not limited solely to base-pairing, that permit the accessory molecule to function as intended.

In the third method of the present invention, the second reporter moiety (located on the accessory molecule) interacts with the first reporter moiety (located on the nucleic acid probe) to produce a detectable signal. Suitable signals include those described above under the subheading “Reporter moieties of the nucleic acid probe”, under the heading “A first method for detecting a single nucleotide polymorphism”. The interaction between the first reporter moiety of the nucleic acid probe and the second reporter moiety of the accessory molecule may result in a detectable signal even when the probe is not hybridized to the target allelic variant of the SNP of interest. Alternatively, one or both of the reporter moieties may individually be capable of producing a detectable signal, preferably a detectable signal that is different from that produced by the interaction of the two reporter moieties, and most preferably a detectable signal that is different from that produced by the interaction of the two reporter moieties when the nucleic acid probe is hybridized to the target allelic variant of the SNP of interest. For example, where the two reporter moieties are two different fluorophores that make up a FRET pair, either or both of the fluorophores may be detected prior to hybridization. In this and analogous cases, it is thus possible to interrogate a system containing the nucleic acid probe and accessory molecule and detect and optionally quantify the amounts of the probe, the accessory molecule, or both the probe and accessory molecule, that are present in the system. A change in the spatial arrangement of the first reporter moiety of the nucleic acid probe relative to the second reporter moiety of the accessory molecule preferably results in a change in the detectable signal produced by the interaction of the two reporter moieties. The change in spatial arrangement may be in terms of the distance between the two reporter moieties, or in terms of an angle.

The second reporter moiety may be located anywhere on the accessory molecule. In some cases, where the accessory molecule includes a nucleic acid sequence or nucleic acid mimic sequence, the second reporter moiety may interrupt the base sequence of the accessory molecule. The second reporter moiety may be a reporter moiety covalently or non-covalently bonded to a location on the accessory molecule. For example, in an accessory molecule that includes a nucleic acid or nucleic acid mimic sequence the second reporter moiety may be a fluorophore covalently or non-covalently bonded to a base of, or elsewhere on, the accessory molecule's nucleic acid or nucleic acid mimic sequence. Alternatively, the second reporter moiety may be a reporter moiety that may be considered an integral or structural part of the accessory molecule. For example, in an accessory molecule that includes a polypeptide, the second reporter moiety may be an amino acid of that polypeptide that is isotopically enriched in a magnetic nucleus (such as ¹⁵N, ¹³C, or ³¹P), which may be detected by heteronuclear magnetic resonance spectroscopy.

The accessory molecule of the third method of the invention may be made by any technique suitable to the composition of the particular accessory molecule, as described above under the subheading “Nucleic acid probe” under the heading “A first method for detecting a single nucleotide polymorphism”. For example, an accessory molecule may include only a nucleic acid (DNA or RNA) or only a nucleic acid mimic, and such an accessory molecule may be made by any suitable DNA, RNA, or nucleic acid mimic synthesis method. The accessory molecule may be a hybrid or chimera, preferably including a nucleic acid (DNA or RNA or both) or a nucleic acid mimic (such as, but not limited to, a peptide nucleic acid) or both; the accessory molecule may further include a polypeptide, a polymer (such as polymeric plastics, silicones, fluorocarbons, polysaccharides, and the like), or a combination thereof. An accessory molecule that is such a hybrid or chimera may be manufactured by a combination of methods, including synthetic, semi-synthetic, enzymatic, recombinant, biological, or a combination thereof.

The methods used to affix the second reporter moiety to the accessory molecule of the third method of the invention depend on the nature of the second reporter moiety and the nature of the accessory molecule, as described above under the subheading “Reporter moieties of the nucleic acid probe” under the heading “A first method for detecting a single nucleotide polymorphism”. Such methods include, for example, covalent cross-linking as well as non-covalent linking methods, isotopic enrichment, or inclusion of a spin label or a heavy atom. Where desired, for example when increased flexibility is needed, a reporter moiety may be affixed using a spacer arm.

The accessory molecule of the third method of the invention may associate with the nucleic acid probe by any suitable interaction or interactions, covalent or non-covalent, not limited solely to base-pairing, that permit the accessory molecule to function as intended. The functions of the accessory molecule of the third method of the invention include the functions of the accessory molecule of the second method of the invention, as described above under “A second method for detecting a single nucleotide polymorphism”. In addition to these functions, the accessory molecule of the third invention serves to bear the second reporter moiety and is thus directly involved in the production of the detectable signal caused by the interaction between the first and second reporter moieties. When the nucleic acid probe is fully hybridized with the target allelic variant of the SNP, this hybridization preferably results in a change in the spatial arrangement of the first reporter moiety relative to the second reporter moiety, and thus changing the detectable signal that is a result of the interaction of the two reporter moieties. More preferably, under a given set of hybridization conditions, the relative change in the spatial arrangement of the first reporter moiety relative to the second reporter moiety, and thus, the relative change in the detectable signal that is a result of the interaction of the two reporter moieties, is different when there is a single base-pairing mismatch between the nucleic acid probe and the target allelic variant of the SNP, than when there is no single base-pairing mismatch between the nucleic acid probe and the target allelic variant of the SNP. Most preferably, under a given set of hybridization conditions, the relative change in the spatial arrangement of the first reporter moiety relative to the second reporter moiety, and thus, the relative change in the detectable signal that is a result of the interaction of the two reporter moieties, is different when there is a single base-pairing mismatch between the first recognition sequence of the nucleic acid probe and the first site of a target allelic variant of the SNP, than when there is no single base-pairing mismatch between the first recognition sequence of the nucleic acid probe and the first site of a target allelic variant of the SNP.

IV. A Nucleic Acid Probe Useful in the First and Second Methods for Detecting a Single Nucleotide Polymorphism

The present invention provides nucleic acid probes useful in the first and second methods for detecting a single nucleotide polymorphism in a sample. A nucleic acid probe useful in the first and second methods of the invention includes a first recognition sequence, a second recognition sequence, a linking element, and a first reporter moiety and a second reporter moiety, such as are described in detail above under the subheadings “Nucleic acid probe”, “First recognition sequence of the nucleic acid probe”, “Second recognition sequence of the nucleic acid probe”, “Linking element of the nucleic acid probe”, and “Reporter moieties of the nucleic acid probe”, all under the heading “A first method for detecting a single nucleotide polymorphism”.

V. A Nucleic Acid Probe Useful in the Third Method for Detecting a Single Nucleotide Polymorphism

The present invention provides nucleic acid probes useful in the third method for detecting a single nucleotide polymorphism in a sample. A nucleic acid probe useful in the third method of the invention includes a first recognition sequence, a second recognition sequence, a linking element, and a first reporter moiety, such as are described in detail above under the subheadings “Nucleic acid probe”, “First recognition sequence of the nucleic acid probe”, “Second recognition sequence of the nucleic acid probe”, “Linking element of the nucleic acid probe”, and “Reporter moieties of the nucleic acid probe”, all under the heading “A first method for detecting a single nucleotide polymorphism”. The first reporter moiety of the nucleic acid probe of the third method of the invention is capable of interacting with a second reporter moiety (located on an accessory molecule of the third method of the invention) to produce a detectable signal, as described above under “A third method for detecting a single nucleotide polymorphism”.

### EXAMPLES

### Example 1

The following example describes the hybridization of a nucleic acid probe to two DNA strands to form a DNA double crossover structure. Unless otherwise noted, all DNA sequences are given in the 5′ to 3′ direction.

Fluorescence Resonance Energy Transfer (FRET)

Fluorescence Resonance Energy Transfer (FRET) is a strongly distance-dependent interaction between a donor fluorophore and an acceptor fluorophore, where excitation energy is transferred from the donor to the acceptor without emission of a photon (R. P. Haugland, “Handbook of Fluorescent Probes and Research Products”, 9^{th} edition, J. Gregory (editor), Molecular Probes, Inc., Eugene, Oreg., USA, 2002, pp 25-26). For FRET to occur, the fluorescence emission spectrum of the donor must overlap the absorption spectrum of the acceptor (FIGURE?), the donor and acceptor transition dipole moments must be approximately parallel, and the donor and acceptor fluorophores must be within a relatively small distance (generally, less than 100 Angstroms) of each other. When the donor and acceptor fluorophores are different, FRET can be observed by detecting the appearance of increased fluorescence by the acceptor or quenching of fluorescence by the donor. When the donor and acceptor fluorophores are the same, FRET can be observed by detecting fluorescence depolarization.

An example of a FRET pair of fluorophores is fluorescein and tetramethylrhodamine. Fluorescein has an excitation maximum wavelength of 494 nanometers, an emission maximum wavelength of 522 nanometers, and an extinction coefficient of 75,000 at 494 nanometers. Tetramethylrhodamine has an excitation maximum wavelength of 556 nanometers, an emission maximum wavelength of 580 nanometers, and an extinction coefficient of 89,000 at 556 nanometers.

The distance at which resonance energy transfer efficiency is 50% is termed the Förster distance or Förster radius, and can be calculated for a given pair of fluorophores by Equation 3:

$${R}_{o} = {[ 8.8 \times {10}^{23} \cdot {κ}^{2} \cdot {n}^{- 4} \cdot {QY}_{D} \cdot J ( λ ) ]}^{\frac{1}{6}} Ångstrøms \qquad ( Equation 3 )$$

where k² is the dipole orientation factor, QY_{D} is the fluorescence quantum yield of the donor in the absence of the acceptor, n is the refractive index, and J(λ) is the spectral overlap between the donor and acceptor. The orientation factor varies between zero and four, but it assumes a numerical value of ⅔ in the Förster equation provided that both fluorophores can participate in unrestricted isotropic motion (dos Remedios and Moens (1995) J. Struct. Biol., 115:175-185). Fluorescein and tetramethylrhodamine molecules have two transition dipole moments, one for the S₀⇄S₁ transition and one for the S₀⇄S₂ transition. The S₀⇄S₂ transitions are relatively much smaller for both molecules, whereas the S₀⇄S₁ transitions have very large magnitudes and account for the two molecules' large extinction coefficients in the visible region as well as their large fluorescence quantum yields (Packard et al. (2000) Prog. Biophys. Mol. Biol., 74:1-35). The Forster distance for fluorescein and tetramethylrhodamine is 5.5 nanometers.

FRET efficiency, or E, can be calculated from Equation 4:

$$E = 1 - \frac{{I}_{DA}}{{I}_{D}} \qquad ( Equation 4 )$$

where I_{DA} is the intensity of the fluorescein peak in the presence of the acceptor, and ID is the intensity of the fluorescein peak in the absence of the acceptor (Andrews and Demidov, “Resonance Energy Transfer”, John Wiley & Sons, Ltd., New York, N.Y., 1999). In the case of fluorescein and tetramethylrhodamine, FRET efficiency should be greater than 0.5 when the fluorophores are at distances less than 5.5 nm, and it should be less than 0.5 when the fluorophores are at distances greater than 5.5 nm.

FRET resonance energy transfer efficiency is dependent on the inverse sixth power of the distance between the donor and the acceptor, and thus FRET is a sensitive measurement of the intermolecular separation between the pair. The distance between the fluorophores can be calculated by Equation 5:

$$E = \frac{{R}_{o}^{6}}{{R}_{o}^{6} + {R}^{6}} \qquad ( Equation 5 )$$

where E is FRET efficiency, R₀ is the Forster distance, and R is the distance between the two fluorophores.

Interactions Between a Nucleic Acid Probe, a Target DNA Strand, and an Accessory Molecule

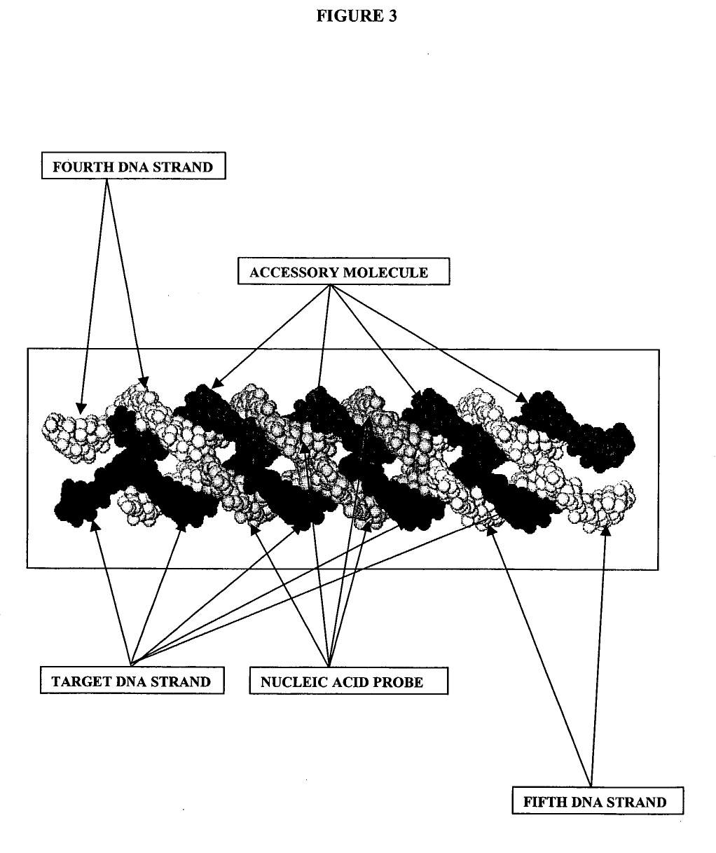

Strands of DNA can, under specific conditions, become linked together to produce a two-dimensional crystal lattice (Winfree et al. (1998) Nature, 394:539-544; Seeman (1998) Ann. Rev. Biophys. Biomol. Struct., 27:225-248). These lattices, also known as nanoarrays, are composed of two repeating units that are often called building blocks, or Block A and Block B. There are several types of these units, but the DAE (double-crossover, antiparallel, even spacing) units were chosen for the synthesis of nanoarrays due to their topology (Cooperativity of DNA Object Self-Assembly, Ava Caudill Dykes, Thesis submitted to the Graduate College of Marshall University in partial fulfillment of the requirements for the degree of Master of Science, Marshall University, Huntington, W. Va., USA, 2001, 72 pp.). The unit examined by this study was the Block A unit, which consists of five strands of DNA as represented in FIG. 3. The annealing processes of a self-assembling model system representing three of the five strands of Block A were examined using fluorescence resonance energy transfer.

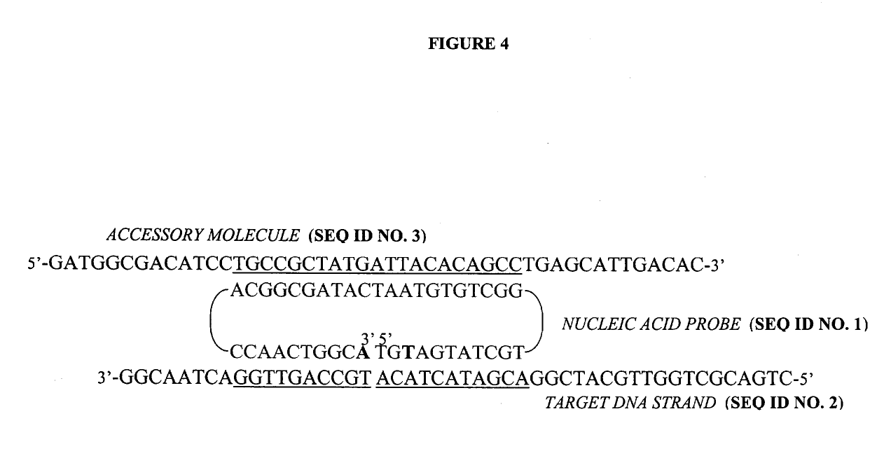

Three different DNA strands were used in this FRET study of a self-assembling DNA double-crossover structure (FIG. 4): (i) a nucleic acid probe having the sequence TGTAGTATCGTGGCTGTGTAATCATAGCGGCACCAACTGGCA (SEQ ID NO. 1); (ii) a target DNA strand having the sequence CTGACGCTGGTTGCATCGGACGATACTACATGCCAGTTGGACTAACGG (SEQ ID NO. 2); and (iii) an accessory molecule consisting of a DNA strand having the sequence GATGGCGACATCCTGCCGCTATGATTACACAGCCTGAGCATTGACAC (SEQ ID NO. 3).

The nucleic acid probe was an oligonucleotide of 42 nucleotides with the sequence TGTAGTATCGTGGCTGTGTAATCATAGCGGCACCAACTGGCA (SEQ ID NO. 1) and including: (a) a first recognition sequence made up of the 11 terminal nucleotides at the 5′ terminus with the sequence TGTAGTATCGT (SEQ ID NO. 4); (b) a second recognition sequence made up of the 10 terminal nucleotides at the 3′ terminus with the sequence CCAACTGGCA (SEQ ID NO. 5); (c) a linking element made up of the intervening 21 nucleotides with the sequence GGCTGTGTAATCATAGCGGCA (SEQ ID NO. 6); (d) a first reporter moiety (a fluorescein molecule attached to the thymine located 3 nucleotides from the 5′ terminus of SEQ ID NO. 1); and (e) a second reporter moiety (a tetramethylrhodamine molecule, attached to the 3′ terminal adenosine of SEQ ID NO. 1). The first recognition sequence (SEQ ID NO. 4) of this nucleic acid probe was complementary to a first target region consisting of the internal sequence ACGATACTACA (SEQ ID NO. 7) located at positions 20 through 30 of the target DNA strand (SEQ ID NO. 2). The second recognition sequence (SEQ ID NO. 5) of this nucleic acid probe was complementary to a second target region consisting of the internal sequence TGCCAGTTGG (SEQ ID NO. 8) located at positions 31 through 40 of the target DNA strand (SEQ ID NO. 2). The linking element (SEQ ID NO. 6) of this nucleic acid probe was complementary to a region consisting of the internal accessory molecule sequence TGCCGCTATGATTACACAGCC (SEQ ID NO. 9) located at positions 14 through 34 of the accessory molecule DNA strand (SEQ ID NO. 3). The linking element (SEQ ID NO. 6) of the nucleic acid probe (SEQ ID NO. 1) was designed to not include a sequence or sequences that are significantly complementary to a sequence or sequences of either or both of the first recognition sequence (SEQ ID NO. 4) and the second recognition sequence (SEQ ID NO. 5) of the nucleic acid probe, and to not include an internal significantly complementary sequence.

Under certain hybridization conditions, the nucleic acid probe, the target DNA strand, and the accessory molecule DNA strand can interact by Watson-Crick nucleotide base pairing and are believed to form a DNA double-crossover structure (for example, as schematically depicted in FIGS. 2C through 2F). In this double-crossover structure, the first recognition sequence (SEQ ID NO. 4) hybridizes to the first target region (SEQ ID NO. 7), the second recognition sequence (SEQ ID NO. 5) hybridizes to the second target region (SEQ ID NO. 8), thus binding the nucleic acid probe to the target DNA strand. In this double-crossover structure, the linking element (SEQ ID NO. 6) hybridizes to the internal accessory molecule sequence SEQ ID NO. 9, thus binding the nucleic acid probe also to the accessory molecule DNA strand.

The two reporter moieties in this example of a nucleic acid probe are capable of interacting to produce a signal through fluorescence resonance energy transfer (FRET), with fluorescein serving as the donor and tetramethylrhodamine as the acceptor, respectively. When self assembling into the hybridized DNA double-crossover structure, the nucleic acid probe changes its configuration, resulting in a change in the spatial arrangement of the first reporter moiety (fluorescein) relative to the second reporter moiety (tetramethylrhodamine), such that the fluorescein and tetramethylrhodamine reporter moieties are brought into closer proximity with each other and FRET can occur. Ideally, minimal FRET efficiency is observed when the probe is unhybridized (for example, free in solution) and maximum FRET efficiency is observed when the hybridized DNA double-crossover structure is completely formed.

General Experimental Conditions

The three DNA reagents (the nucleic acid probe (SEQ ID NO. 1), the target DNA strand (SEQ ID NO. 2), and the accessory molecule DNA strand (SEQ ID NO. 3)) were synthesized by MWG Biotech, Inc. (High Point, N.C., USA) or by Integrated DNA Technologies, Inc (Coralville, Iowa, USA). Fluorescent labeling of the nucleic acid probe was performed with fluorescein-dt (a modified base wherein fluorescein is attached to position 5 of the thymine ring by a six-carbon spacer arm, permitting insertion of fluorescein at an internal position in a nucleotide sequence) and carboxytetramethylrhodamine (TAMRA™): the fluorescein label was attached to the thymine located 3 nucleotides from the 5′ terminus of the nucleic acid probe (SEQ ID NO. 1), and the tetramethylrhodamine label to the 3′ terminal adenosine of the nucleic acid probe (SEQ ID NO. 1). Each dry DNA reagent was individually dissolved in N-2-hydroxyethylpiperazine-N′-2-ethanesulfonic acid (HEPES) buffer, pH 7, at a concentration of 100 micromoles per liter, and stored in a −20 degrees Celsius freezer. This buffer exhibits relatively small changes in pH due to temperature extremes, thus stabilizing the DNA reagents during frozen storage and during annealing experiments. When needed, samples of the DNA reagents were thawed, diluted as necessary with HEPES buffer, and mixed using a Fisher Vortex Genie 2 (Fisher Scientific catalogue number 12-812, manufactured by Scientific Industries, Bohemia, N.Y., USA).

Temperature-dependent fluorescence experiments were conducted using the DNA reagents individually (single strand experiments) or in combination (double strand experiments). For each experiment, samples of the DNA reagents were individually diluted to a final concentration of 0.4 micromoles per liter for each DNA reagent. Thus, the final total DNA concentration for the single strand experiments was 0.4 micromoles per liter, and for the double strand experiments, 0.8 micromoles per liter. Experiments were performed in a final total volume of 2 milliliters. DNA dilutions were performed in disposable, acrylic, 3.5-milliliter fluorimeter cuvettes (Spectrocell, Inc., Oreland, Pa., USA). The final DNA solutions were mixed again on the Fisher Vortex Genie 2, and then were examined using a Spex Fluorolog III fluorimeter (Jobin Yvon, Inc., Edison, N.J., USA) operated with a scan range of 498-648 nanometers, an excitation wavelength of 480 nanometers, an integration time of 0.1 second, scanning increments of 1 nanometer, and with excitation and emission slit widths of 5 nanometers.

For each temperature-dependent fluorescence experiment, an initial scan of the DNA solution at room temperature was taken, and then an annealing process was run, wherein the DNA solution was heated to an initial temperature of 90 degrees Celsius followed by gradual cooling over a period of about 2 hours to a final temperature of 20 degrees Celsius. Temperature-dependent scans were taken at 10-degree intervals with a tolerance of 0.2 degrees Celsius and an equilibration time of 1 minute. After the scan at 20 degrees Celsius, the DNA solution was allowed to return to room temperture and a final scan was taken. Thus, each experiment consisted of ten fluorometric scans. The spectra produced were split into single files and converted into a Microsoft Excel compatible format for further analysis.

Concentration Experiments

The nucleic acid probe (SEQ ID NO. 1) used in these experiments was synthesized by MWG Biotech, Inc. (High Point, N.C., USA), and the target DNA strand (SEQ ID NO. 2) and the accessory molecule DNA strand (SEQ ID NO. 3) were synthesized by Integrated DNA Technologies, Inc (Coralville, Iowa, USA). Stock solutions were thawed and mixed on a Fisher Vortex Genie 2. Accessory molecule DNA strand (9.6 microliters of stock solution), nucleic acid probe (8 microliters of stock solution), target DNA strand (8 microliters of stock solution), and 1975 microliters of HEPES buffer were added to a fluorimeter cuvette to give a final solution containing 0.48 micromoles per liter of accessory molecule DNA strand, 0.4 micromoles per liter of nucleic acid probe, and 0.4 micromoles per liter of target DNA strand, or a final total DNA concentration of 1.28 micromoles per liter. These concentrations are consistent with guidelines for DNA nanoarray synthesis studies (Winfree et al. (1998), Nature, 394:539-544). The mixed DNA solution was scanned at room temperature in a SPEX Fluorolog m fluorimeter. A series of ten three-fold dilutions of the mixed DNA solution were made with HEPES buffer in fluorimeter cuvettes, and these diluted samples (0.43, 0.14, 0.047, 0.016, 5.3×10⁻³, 1.8×10⁻³, 5.9×10⁻⁴, 2.0×10⁻⁴, 6.5×10⁻⁵, and 2.2×10⁻⁵ micromoles per liter, respectively) were also scanned at room temperature. Acceptable signal-to-noise ratios for detection were observed in the spectra of samples with total DNA concentrations of 0.43 micromoles per liter or greater. In addition, the ratio of fluorescein fluorescence intensity relative to tetramethylrhodamine fluorescence intensity was examined, and at lower concentrations a contribution to the signal in the area of tetramethylrhodamine fluorescence emission was observed as the result of the Raman band for water, a result of Raman scattering, which appears at 567 nanometers when water is excited at 480 nanometers. It may be predicted that a limit to this particular detection system is the Raman band for water, a relatively low-intensity signal (about 3 orders of magnitude less than that of tetramethylrhodamine emission observed for the sample with a total DNA concentration of 1.28 micromoles per liter). Where the water Raman signal is relatively large, it may interfere with accurate measurements of tetramethylrhodamine fluorescence emission at 580 nanometers.

Temperature-Dependent Fluorescence Experiments

To investigate the behaviour of the three-strand self-assembling DNA double-crossover structure, two independent sets of temperature-dependent fluorescence experiments were conducted with the nucleic acid probe (SEQ ID NO. 1), the target DNA strand (SEQ ID NO. 2), and the accessory molecule DNA strand (SEQ ID NO. 3). All DNA used in the first set of experiments was synthesized by MWG Biotech, Inc. (High Point, N.C., USA). All DNA used in the second set of experiments was synthesized by Integrated DNA Technologies, Inc (Coralville, Iowa, USA). Unless otherwise noted, all additional experimental conditions were as given above in “General experimental conditions”.

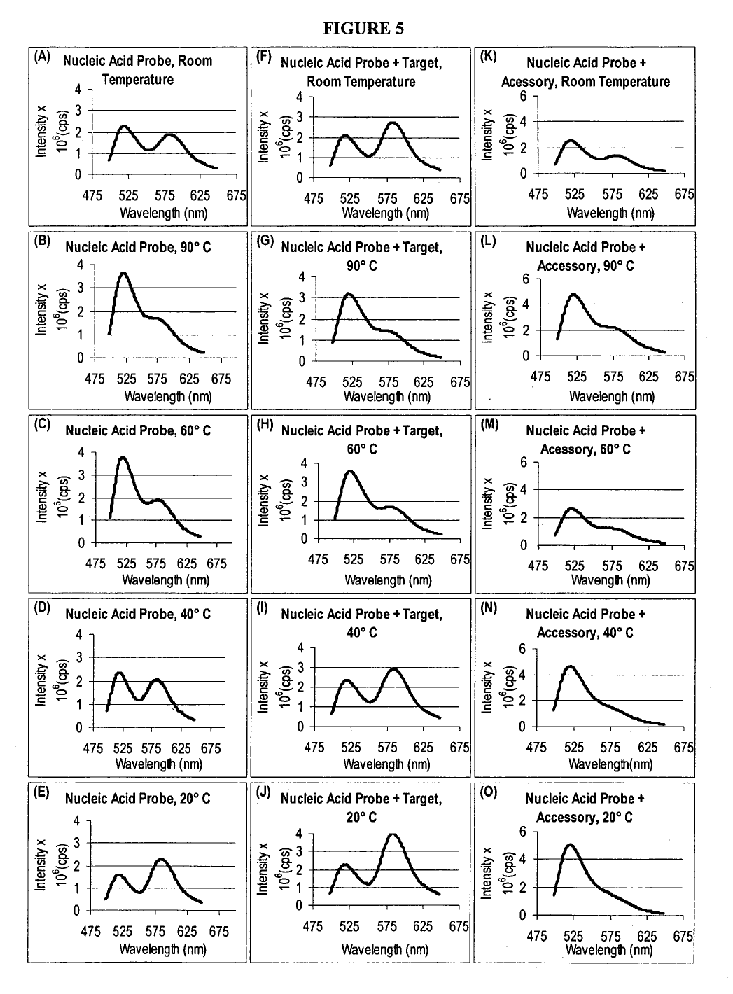

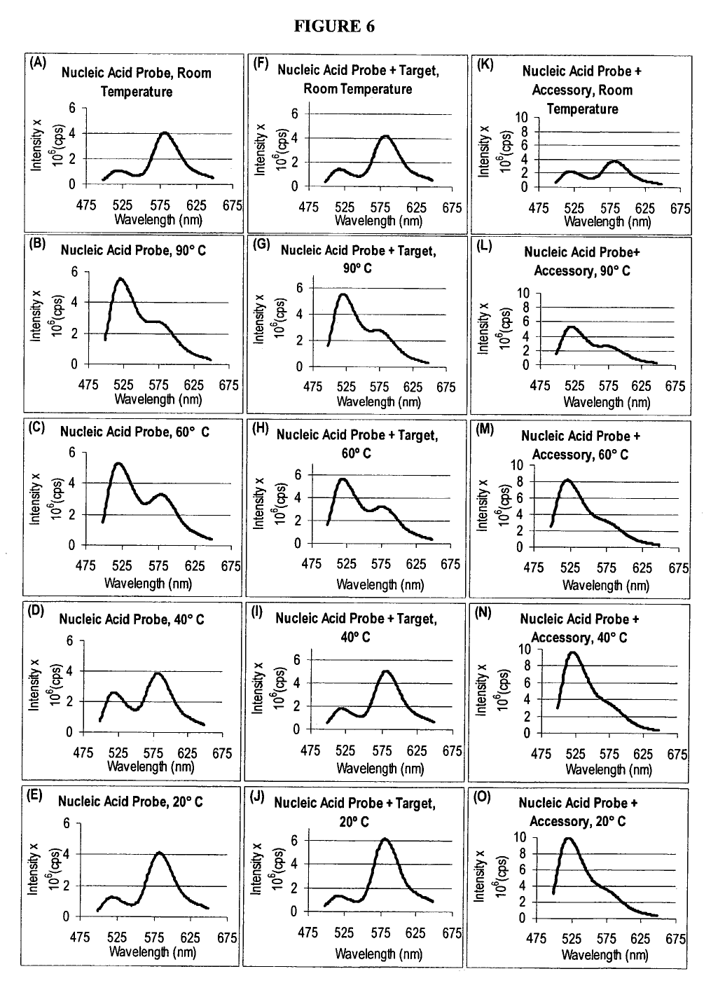

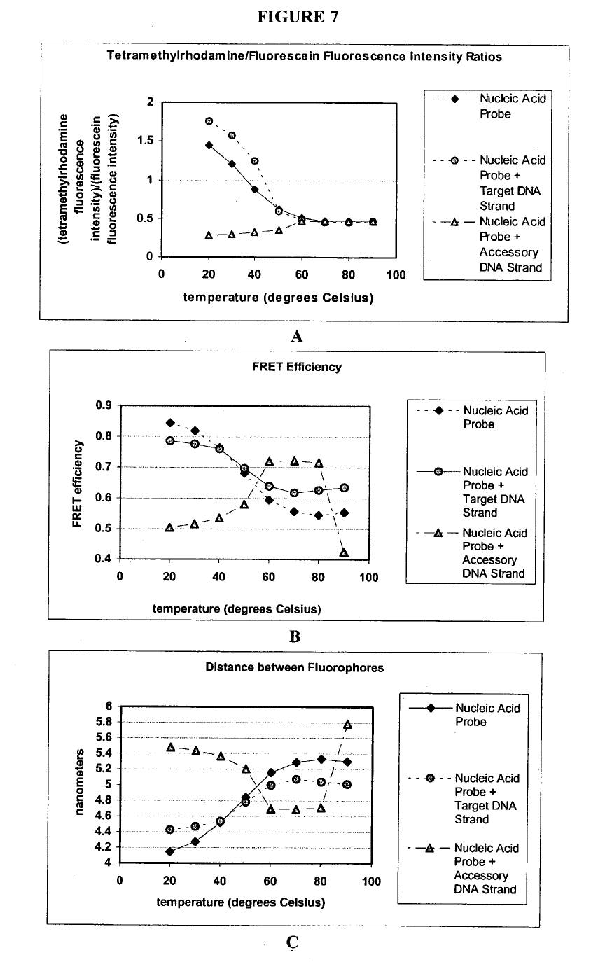

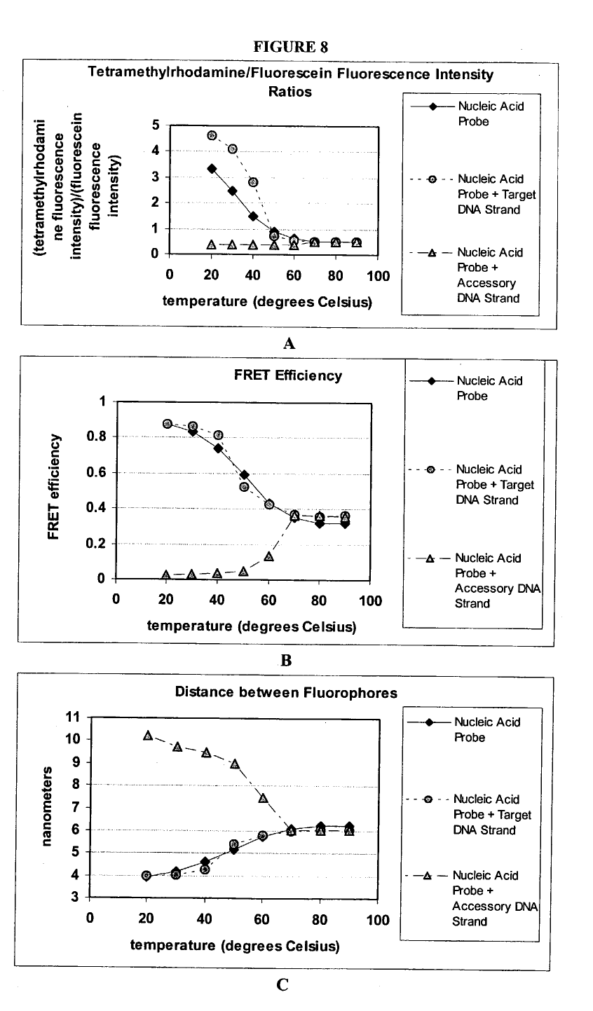

Representative fluorescence spectra from the first set of experiments are shown in FIG. 5, and from the second set of experiments in FIG. 6. The collected spectral data were used to calculate the ratio of tetramethylrhodamine intensity to fluorescein intensity, the FRET efficiency, and the distance between the two fluorophores. These results are given in Table 1. Temperature-dependent plots of these calculated values are shown for the first set of experiments in FIG. 7 and for the second set of experiments in FIG. 8.

TABLE 1

| | First set of experiments | | | Second set of experiments | | |
|---|---|---|---|---|---|---|
| | | | Nucleic | | | Nucleic |
| | | Nucleic | acid | | Nucleic | acid |
| | | acid | probe + | | acid | probe + |
| Temper- | | probe + | accessory | | probe + | accessory |
| ature | Nucleic | target | molecule | Nucleic | target | molecule |
| (degrees | acid | DNA | DNA | acid | DNA | DNA |
| Celsius) | probe | strand | strand | probe | strand | strand |
| | Tetramethylrhodamine/Fluorescein | | | | | |
| | Fluorescence Intensity Ratio | | | | | |
| 20 | 1.444 | 1.760 | 0.288 | 3.338 | 4.626 | 0.376 |
| 30 | 1.200 | 1.580 | 0.291 | 2.476 | 4.116 | 0.373 |
| 40 | 0.875 | 1.242 | 0.322 | 1.501 | 2.814 | 0.377 |
| 50 | 0.623 | 0.599 | 0.349 | 0.897 | 0.736 | 0.378 |
| 60 | 0.504 | 0.475 | 0.464 | 0.621 | 0.573 | 0.403 |
| 70 | 0.471 | 0.449 | 0.468 | 0.525 | 0.523 | 0.511 |
| 80 | 0.465 | 0.450 | 0.465 | 0.505 | 0.505 | 0.502 |
| 90 | 0.468 | 0.462 | 0.469 | 0.500 | 0.502 | 0.502 |
| | FRET Efficiency | | | | | |
| 20 | 0.845 | 0.786 | 0.505 | 0.879 | 0.875 | 0.024 |
| 30 | 0.819 | 0.776 | 0.516 | 0.836 | 0.866 | 0.031 |
| 40 | 0.764 | 0.761 | 0.536 | 0.740 | 0.816 | 0.037 |
| 50 | 0.682 | 0.698 | 0.581 | 0.593 | 0.526 | 0.051 |
| 60 | 0.595 | 0.640 | 0.721 | 0.432 | 0.426 | 0.138 |
| 70 | 0.558 | 0.618 | 0.722 | 0.351 | 0.37 | 0.364 |
| 80 | 0.546 | 0.628 | 0.716 | 0.323 | 0.359 | 0.362 |
| 90 | 0.554 | 0.636 | 0.425 | 0.320 | 0.363 | 0.359 |
| | Distance between Fluorophores | | | | | |
| 20 | 4.147 | 4.428 | 5.482 | 3.954 | 3.977 | 10.207 |
| 30 | 4.276 | 4.469 | 5.440 | 4.191 | 4.032 | 9.746 |
| 40 | 4.521 | 4.536 | 5.368 | 4.619 | 4.290 | 9.456 |
| 50 | 4.844 | 4.784 | 5.208 | 5.167 | 5.404 | 8.965 |
| 60 | 5.160 | 4.997 | 4.696 | 5.755 | 5.780 | 7.465 |
| 70 | 5.290 | 5.076 | 4.690 | 6.093 | 6.009 | 6.038 |
| 80 | 5.334 | 5.042 | 4.713 | 6.221 | 6.059 | 6.045 |
| 90 | 5.303 | 5.012 | 5.785 | 6.237 | 6.040 | 6.057 |

Representative fluorescence spectra of the nucleic acid probe (SEQ ID NO. 1) from the first set of experiments are shown in FIG. 5. These show that fluorescence emission of both the fluorescein (emission maximum at 522 nanometers) and of the tetramethylrhodamine (emission maximum at 580 nanometers) reporter moieties was observed at the initial room temperature scan (FIG. 5A), indicating that at least some of the fluorescein reporter moieties were within FRET distance of at least some of the rhodamine reporter moieties. The observed FRET transfer could be intramolecular or intermolecular. Self-complementary segments within the nucleic acid probe sequence possibly exist and could have resulted in two or more strands interacting or binding to each other in various intramolecular or intermolecular configurations where a fluorescein reporter moiety is brought within Förster distance of a tetramethylrhodamine reporter moiety. For example, tetramethylrhodamine on one molecule of the nucleic acid probe could have accepted FRET from fluorescein located on a second molecule of the nucleic acid probe, or a single molecule of the nucleic acid probe might have adopted a hairpin configuration in which intramolecular FRET occurred.

Upon heating the nucleic acid probe to 90 degrees Celsius, the tetramethylrhodamine emission substantially decreased whereas the fluorescein emission substantially increased, both observations indicating that FRET had decreased (FIG. 5B). As the solution was cooled, the ratio of tetramethylrhodamine intensity to fluorescein intensity progressively increased from 0.47 to 1.4 (FIGS. 5C-5E, and FIG. 7A), FRET efficiency progressively increased from 0.55 to 0.84 (FIG. 7B), and the distance between the two fluorophores decreased from 5.3 nanometers to 4.1 nanometers (FIG. 7C).

Representative fluorescence spectra of the nucleic acid probe (SEQ ID NO. 1) and target DNA strand (SEQ ID NO. 2) from the first set of experiments are shown in FIG. 5. In this double-strand experiment, some FRET was observed at room temperature (FIG. 5F). Upon heating to 90 degrees Celsius, FRET decreased (FIG. 5G). As the solution was cooled, the ratio of tetramethylrhodamine intensity to fluorescein intensity progressively increased from 0.46 to 1.8 (FIGS. 5H-5J, and FIG. 7A), FRET efficiency progressively increased from 0.64 to 0.79 (FIG. 7B), and the distance between the two fluorophores decreased from 5.0 nanometers to 4.4 nanometers (FIG. 7C). As the solution was cooled, the ratios of tetramethylrhodamine intensity to fluorescein intensity for the nucleic acid probe and target DNA strand mixture was greater than those for the nucleic acid probe alone at a given temperature of 40 degrees Celsius or lower (FIG. 7A). These observations support the occurrence of the expected association of the nucleic acid probe and the target DNA strand as depicted in FIG. 4, where the first recognition sequence (SEQ ID NO. 4) of the nucleic acid probe hybridizes to the first target region (SEQ ID NO. 7) of the target DNA strand, and the second recognition sequence (SEQ ID NO. 5) of the nucleic acid probe hybridizes to the second target region (SEQ ID NO. 8) of the target DNA strand. The target DNA strand is believed to bring the nucleic acid probe's fluorophores within the Forster distance more effectively than seen for the nucleic acid probe alone.

Another double-strand experiment was conducted using the nucleic acid probe (SEQ ID NO. 1) and the accessory molecule DNA strand (SEQ ID NO. 3). Representative fluorescence spectra from the first set of experiments are shown in FIG. 5. The expected association of the nucleic acid probe and the accessory molecule DNA strand is depicted in FIG. 4, where the linking element (SEQ ID NO. 6) of the nucleic acid probe hybridizes to the internal accessory molecule sequence SEQ ID NO. 9 of the accessory molecule DNA strand. In this double-stranded experiment, a small amount of FRET was observed at room temperature (FIG. 5K) that was less than seen for the nucleic acid probe alone (FIG. 5A). Upon heating to 90 degrees Celsius, FRET decreased (FIG. 5L). As the solution was cooled, the ratio of tetramethylrhodamine intensity to fluorescein intensity progressively decreased from 0.47 to 0.29 (FIGS. 5M-5O, and FIG. 7A), in sharp contrast to the observations for the nucleic acid probe alone or the nucleic acid probe and target DNA strand. FRET efficiency decreased overall from 0.72 at 80 degrees Celsius to 0.51 at 20 degrees Celsius (FIG. 7B), and the distance between the two fluorophores increased overall from 4.7 nanometers at 80 degrees Celsius to 5.5 nanometers at 20 degrees Celsius (FIG. 7C). These observations indicated that the accessory molecule DNA strand did bind the nucleic acid probe in the predicted configuration, decreasing the ability of the two reporter moieties to interact in a manner that causes FRET, and thus decreasing the amount of false positive or background signal (FIG. 5K).

A second set of experiments were performed using DNA from a different manufacturer (Integrated DNA Technologies, Coralville, Iowa, USA). The dry DNA was dissolved in double distilled, autoclaved water to give about 50 micromoles per liter stock solutions based on the manufacturer's concentrations predictions, and these stock solutions were stored in a refrigerator. The absorbance at 260 nanometers of each stock solution was measured with a Spectronic Genesys 5 spectrophotometer (catalogue number 336008, Thermo Spectronic, Rochester, N.Y., USA), and the true concentrations calculated to be 52.815 micromoles per liter for the nucleic acid probe (SEQ ID NO. 1), 55.994 micromoles per liter for the target DNA strand (SEQ ID NO. 2), and 52.386 micromoles per liter for the accessory molecule DNA strand (SEQ ID NO. 3). Dilutions were made with HEPES buffer in semi-micro (1.5 milliliter), disposable, methacrylate fluorimeter cuvettes (catalogue number 14-385-938, Fisher Scientific, USA).

Representative fluorescence spectra from the second set of experiments are shown in FIG. 6. The calculated ratio of tetramethylrhodamine intensity to fluorescein intensity, the FRET efficiency, and the distance between the two fluorophores are given in Table 1. Temperature-dependent plots of these calculated values are shown in FIG. 8.

Temperature-dependent fluorescent spectra were collected for the nucleic acid probe (SEQ ID NO. 1) at a concentration of 0.4225 micromoles per liter. Representative fluorescence spectra of the nucleic acid probe (SEQ ID NO. 1) from the second set of experiments are shown in FIG. 6. At room temperature (FIG. 6A), the amount of FRET observed in this experiment was greater than that seen in the first set of experiments, but as the solution was heated and then cooled, the spectral behaviour was similar to that seen in the first set of experiments. Upon heating the nucleic acid probe to 90 degrees Celsius, the tetramethylrhodamine emission substantially decreased whereas the fluorescein emission substantially increased, both observations indicating that FRET had decreased (FIG. 6B). As the solution was cooled, the ratio of tetramethylrhodamine intensity to fluorescein intensity progressively increased from 0.50 to 3.3 (FIGS. 6C-6E, and FIG. 8A), FRET efficiency progressively increased from 0.32 to 0.88 (FIG. 8B), and the distance between the two fluorophores decreased from 6.2 nanometers to 4.0 nanometers (FIG. 8C), suggesting either intermolecular or intramolecular FRET was occurring as the strands annealed.

Representative fluorescence spectra of the nucleic acid probe (SEQ ID NO. 1) and target DNA strand (SEQ ID NO. 2) from the second set of experiments are shown in FIG. 6. The spectral behaviour was again similar to that seen in the first set of experiments. In this double-strand experiment, some FRET was again observed at room temperature (FIG. 6F). Upon heating to 90 degrees Celsius, FRET decreased (FIG. 6G). As the solution was cooled, the ratio of tetramethylrhodamine intensity to fluorescein intensity progressively increased from 0.50 to 4.6 (FIGS. 6H-6J, and FIG. 8A), FRET efficiency progressively increased from 0.36 to 0.87 (FIG. 8B), and the distance between the two fluorophores decreased from 6.0 nanometers to 4.0 nanometers (FIG. 8C). The FRET efficiency observed in the second set of experiments was relatively greater at room temperature prior to heating and at temperatures of 40 degrees Celsius or lower after cooling than in the first set of experiments. As the solution was cooled, the ratios of tetramethylrhodamine intensity to fluorescein intensity for the nucleic acid probe and target DNA strand mixture was greater than those for the nucleic acid probe alone at a given temperature of 40 degrees Celsius or lower (FIG. 8A). These observations again support the occurrence of the expected association of the nucleic acid probe and the target DNA strand as depicted in FIG. 4, where the first recognition sequence (SEQ ID NO. 4) of the nucleic acid probe hybridizes to the first target region (SEQ ID NO. 7) of the target DNA strand, and the second recognition sequence (SEQ ID NO. 5) of the nucleic acid probe hybridizes to the second target region (SEQ ID NO. 8) of the target DNA strand. The target DNA strand is believed to bring the nucleic acid probe's fluorophores within the Forster distance more effectively than seen for the nucleic acid probe alone.

A second set of double-strand experiments was conducted using the nucleic acid probe (SEQ ID NO. 1) and the accessory molecule DNA strand (SEQ ID NO. 3). Representative fluorescence spectra from the second set of experiments are shown in FIG. 6. In this double-strand experiment, the amount of FRET observed at room temperature (FIG. 6K) was again greater than that seen in the first set of experiments, but again was lower for the nucleic acid probe and accessory molecule (FIG. 6K) than for the nucleic acid probe alone (FIG. 6A). Upon heating to 90 degrees Celsius, FRET decreased (FIG. 6L). As the solution was cooled, the ratio of tetramethylrhodamine intensity to fluorescein intensity progressively decreased from 0.50 to 0.38 (FIGS. 6M-6O, and FIG. 8A), in sharp contrast to the observations for the nucleic acid probe alone or the nucleic acid probe and target DNA strand. FRET efficiency decreased from 0.36 to 0.02 (FIG. 8B), and the distance between the two fluorophores increased overall from 6.1 nanometers to 10.2 nanometers (FIG. 8C). As had been seen in the first set of experiments, these observations again indicated that the accessory molecule DNA strand did bind the nucleic acid probe in the predicted configuration, decreasing the ability of the two reporter moieties to interact in a manner that causes FRET in the nucleic acid probe in the absence of the target DNA strand, and thus decreasing the amount of false positive or background signal (FIG. 6K).

The annealing between the nucleic acid probe and the target DNA strand, or between the nucleic acid probe and the accessory molecule DNA strand, was increased in the second set of experiments relative to the first set, possibly due to an improved stoichiometry between reactants in the solutions used in the second set of experiments.

### Example 2

The following example describes hybridization of a nucleic acid probe to a target DNA strand and to an accessory molecule strand. General experimental conditions are as described above in Example 1. Unless otherwise noted, all DNA sequences are given in the 5′ to 3′ direction.

A triple-strand experiment uses a DNA solution that includes the nucleic acid probe (SEQ ID NO. 1), the target DNA strand (SEQ ID NO. 2), and the accessory molecule DNA strand (SEQ ID NO. 3), each at a concentration of 0.4 micromoles per liter for a total DNA concentration of about 1.2 micromoles per liter. The order of addition of concentrated DNA stock solutions to the diluted experimental mixture is the nucleic acid probe, followed by the accessory molecule DNA strand, and finally the target DNA strand. The nucleic acid probe is expected to hybridize first to the accessory molecule DNA strand, which binds the nucleic acid probe in the predicted configuration, decreasing the ability of the two reporter moieties to interact in a manner that causes FRET in the nucleic acid probe in the absence of the target DNA strand. Upon addition of the target DNA strand, the three strands interact by Watson-Crick nucleotide base pairing and are believed to form a DNA double-crossover structure, wherein the first recognition sequence (SEQ ID NO. 4) hybridizes to the first target region (SEQ ID NO. 7), the second recognition sequence (SEQ ID NO. 5) hybridizes to the second target region (SEQ ID NO. 8), and the linking element (SEQ ID NO. 6) hybridizes to the internal accessory molecule sequence SEQ ID NO. 9, thus binding the nucleic acid probe to both the target DNA strand and to the accessory molecule DNA strand.

At room temperature, some FRET is observed in the triple-strand solution's fluorescence spectrum, as is true for the fluorescence spectra for both the single-strand experiments and double-strand experiments (see Example 1). The amount of FRET observed at room temperature is relatively lower than in that observed in the single-strand experiment of Experiment 1, again indicating that the linking element (SEQ ID NO. 6) of the nucleic acid probe hybridizes to the internal accessory molecule sequence SEQ ID NO. 9, and thus the accessory molecule DNA strand may help to maintain the two reporter moieties at a distance large enough to prevent significant intramolecular FRET from occurring, and thus helps to minimize false positive signals. When the triple-strand mixture is heated to 90 degrees Celsius, the amount of FRET decreases, and then increases progressively as the solution is cooled to 20 degrees Celsius. As the triple-strand mixture is cooled, the ratios of tetramethylrhodamine intensity to fluorescein intensity are relatively greater than those observed for the nucleic acid probe alone, or for either the nucleic acid probe and accessory molecule or the nucleic acid probe and target DNA strand experiments of Example 1 at a given temperature of 40 degrees Celsius or lower, indicating that in the triple-strand experiment, the accessory molecule DNA strand enhances the overall hybridization between the nucleic acid probe and the target DNA strand. This enhancement may be due to the accessory molecule DNA strand limiting the range of internal motions of the nucleic acid probe or limiting the range of locations on the target DNA strand with which the nucleic acid probe can interact. Preferably, the accessory molecule reduces the background or false positive noise in such a way as to increase the difference in signal between the nucleic acid probe complexed to the accessory molecule, and the nucleic acid probe complexed to the accessory molecule and hybridized to the target.

### Example 3

The following example describes the experiments in which hybridization of a nucleic acid probe to four different samples of DNA, differing only in a single nucleotide polymorphism (SNP), yields observable differences in signals. This example demonstrates the sensitivity of the probe structure to single base mismatch between probe and target, and hence, to SNPs. Unless otherwise noted, all DNA sequences are given in the 5′ to 3′ direction.

Hybridization of a Nucleic Acid Probe to Different Target Allelic Variants of a Single Nucleotide Polymorphism

Strands of DNA can, under specific conditions, become linked together through Watson-Crick base pairing. The hybridization process between a nucleic acid probe and different target allelic variants of a single nucleotide polymorphism (SNP) was examined using fluorescence resonance energy transfer. These experiments demonstrated the sensitivity of the probe to a mismatch between the first recognition sequence of the nucleic acid probe and a first site of a target allelic variant of an SNP.

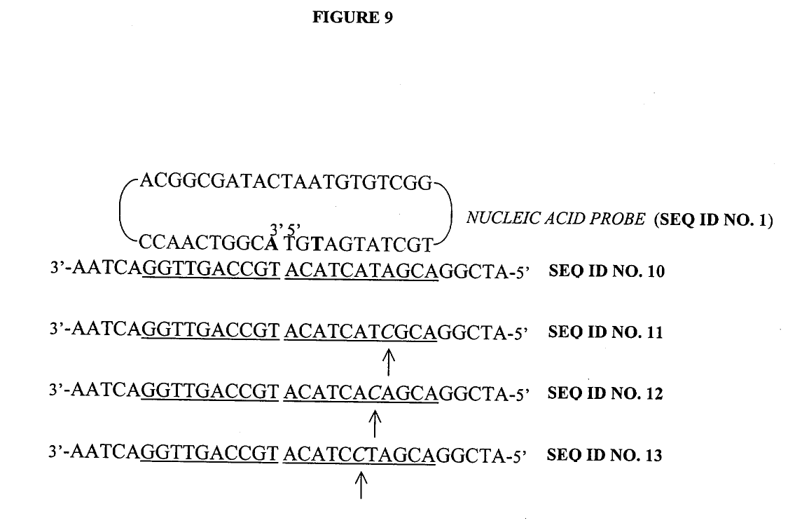

Five DNA constructs were used in this FRET study of a self-assembling DNA double crossover duplex (FIG. 9): (i) a nucleic acid probe identical to that used in Example 1; and (ii) four different target DNA strands (SEQ ID NO. 10, SEQ ID NO. 11, SEQ ID NO. 12, and SEQ ID NO. 13). The first target DNA strand (SEQ ID NO. 10) is capable of base-pairing with no mismatches with the nucleic acid probe (SEQ ID NO. 1), and represents a target allelic variant of an SNP that base-pairs perfectly with the nucleic acid probe (SEQ ID NO. 1). The second, third, and fourth target DNA strands (SEQ ID NO. 11, SEQ ID NO. 12, and SEQ ID NO. 13) are each capable of base-pairing with a single base-pairing mismatch with the nucleic acid probe (SEQ ID NO. 1), and represent three different target allelic variants of an SNP. The mismatched base is located at a different locus in each of the second, third, and fourth target DNA strands (SEQ ID NO. 11, SEQ ID NO. 12, and SEQ ID NO. 13).

The nucleic acid probe was an oligonucleotide of 42 nucleotides with the sequence TGTAGTATCGTGGCTGTGTAATCATAGCGGCACCAACTGGCA (SEQ ID NO. 1) and including: (a) a first recognition sequence made up of the 11 terminal nucleotides at the 5′ terminus with the sequence TGTAGTATCGT (SEQ ID NO. 4); (b) a second recognition sequence made up of the ten terminal nucleotides at the 3′ terminus with the sequence CCAACTGGCA (SEQ ID NO. 5); (c) a linking element made up of the intervening 21 nucleotides with the sequence GGCTGTGTAATCATAGCGGCA (SEQ ID NO. 6); (d) and a first reporter moiety (a fluorescein molecule attached to the thymine located 3 nucleotides from the 5′ terminus of SEQ ID NO. 1) and a second reporter moiety (a tetramethylrhodamine molecule, attached to the 3′ terminal adenosine of SEQ ID NO. 1).

The first target DNA strand was an oligonucleotide of 31 nucleotides with the sequence ATCGGACGATACTACATGCCAGTTGGACTAA (SEQ ID NO. 10), and the two internal sequences: (a) ACGATACTACA (SEQ ID NO. 7), which represents a first site of a target allelic variant of an SNP, and is capable of base-pairing with no mismatch with the fluorescein-labelled first recognition sequence (SEQ ID NO. 4) of the nucleic acid probe (SEQ ID NO. 1), and (b) TGCCAGTTGG (SEQ ID NO. 8), which represents a second site of a target allelic variant of an SNP, and which is capable of base-pairing with no mismatch with the tetramethylrhodamine-labelled second recognition sequence (SEQ ID NO. 5) of the nucleic acid probe (SEQ ID NO. 1).

The second target DNA strand was an oligonucleotide of 31 nucleotides with the sequence ATCGGACGCTACTACATGCCAGTTGGACTAA (SEQ ID NO. 11), and the two internal sequences: (a) ACGCTACTACA (SEQ ID NO. 14), which represents a first site of a target allelic variant of an SNP, and is capable of base-pairing with a single base-pairing mismatch with the fluorescein-labelled first recognition sequence (SEQ ID NO. 4) of the nucleic acid probe (SEQ ID NO. 1), and (b) TGCCAGTTGG (SEQ ID NO. 8), which represents a second site of a target allelic variant of an SNP, and which is capable of base-pairing with no mismatch with the tetramethylrhodamine-labelled second recognition sequence (SEQ ID NO. 5) of the nucleic acid probe (SEQ ID NO. 1). The locus of the single base-pairing mismatch is at the ninth nucleotide (reading in the 5′ to 3′ direction), a cytosine, of SEQ ID NO. 11, which is mismatched to the eighth nucleotide (reading in the 5′ to 3′ direction), a thymine, of the first recognition sequence (SEQ ID NO. 4) of the nucleic acid probe (SEQ ID NO. 1). Thus, this mismatch is five nucleotides distant from the thymine bearing the fluorescein moiety in SEQ ID NO. 1.

The third target DNA strand was an oligonucleotide of 31 nucleotides with the sequence ATCGGACGACACTACATGCCAGTTGGACTAA (SEQ ID NO. 12), and the two internal sequences: (a) ACGACACTACA (SEQ ID NO. 15), which represents a first site of a target allelic variant of an SNP, and is capable of base-pairing with a single base-pairing mismatch with the fluorescein-labelled first recognition sequence (SEQ ID NO. 4) of the nucleic acid probe (SEQ ID NO. 1), and (b) TGCCAGTTGG (SEQ ID NO. 8), which represents a second site of a target allelic variant of an SNP, and which is capable of base-pairing with no mismatch with the tetramethylrhodamine-labelled second recognition sequence (SEQ ID NO. 5) of the nucleic acid probe (SEQ ID NO. 1). The locus of the single base-pairing mismatch is at the tenth nucleotide (reading in the 5′ to 3′ direction), a cytosine, of SEQ ID NO. 11, which is mismatched to the seventh nucleotide (reading in the 5′ to 3′ direction), an adenine, of the first recognition sequence (SEQ ID NO. 4) of the nucleic acid probe (SEQ ID NO. 1). Thus, this mismatch is four nucleotides distant from the thymine bearing the fluorescein moiety in SEQ ID NO. 1.

The fourth target DNA strand was an oligonucleotide of 31 nucleotides with the sequence ATCGGACGATCCTACATGCCAGTTGGACTAA (SEQ ID NO. 13), and the two internal sequences: (a) ACGATCCTACA (SEQ ID NO. 16), which represents a first site of a target allelic variant of an SNP, and is capable of base-pairing with a single base-pairing mismatch with the fluorescein-labelled first recognition sequence (SEQ ID NO. 4) of the nucleic acid probe (SEQ ID NO. 1), and (b) TGCCAGTTGG (SEQ ID NO. 8), which represents a second site of a target allelic variant of an SNP, and which is capable of base-pairing with no mismatch with the tetramethylrhodamine-labelled second recognition sequence (SEQ ID NO. 5) of the nucleic acid probe (SEQ ID NO. 1). The locus of the single base-pairing mismatch is at the eleventh nucleotide (reading in the 5′ to 3′ direction), a cytosine, of SEQ ID NO. 11, which is mismatched to the sixth nucleotide (reading in the 5′ to 3′ direction), a thymine, of the first recognition sequence (SEQ ID NO. 4) of the nucleic acid probe (SEQ ID NO. 1). Thus, this mismatch is three nucleotides distant from the thymine bearing the fluorescein moiety in SEQ ID NO. 1.

The linking element (SEQ ID NO. 6) of the nucleic acid probe (SEQ ID NO. 1) was designed to not include a sequence or sequences that are significantly complementary to a sequence or sequences of either or both of the first recognition sequence (SEQ ID NO. 4) and the second recognition sequence (SEQ ID NO. 5) of the nucleic acid probe, to not include an internal significantly complementary sequence, and to not include a sequence that is significantly complementary to any part of the four target DNA strands.

Under appropriate hybridization conditions, the first target DNA strand (SEQ ID NO. 10) may be expected to interact by Watson-Crick base-pairing with the nucleic acid probe (SEQ ID NO. 1) to form a complex, wherein the first recognition sequence (SEQ ID NO. 4) of the nucleic acid probe hybridizes to the representative first site of a target allelic variant of an SNP (SEQ ID NO. 7) and the second recognition sequence (SEQ ID NO. 5) of the nucleic acid probe hybridizes to the representative second site of a target allelic variant of an SNP (SEQ ID NO. 8), respectively, of the first target DNA strand. The linking element (SEQ ID NO. 6) of the nucleic acid probe may be expected to remain significantly unhybridized. Upon hybridization, the nucleic acid probe changes its configuration whereby the 5′ terminus and the 3′ terminus of the nucleic acid probe are brought into near proximity with each other. This results in a change in the spatial arrangement of the first reporter moiety (fluorescein) relative to the second reporter moiety (tetramethylrhodamine), such that the fluorescein and tetramethylrhodamine reporter moieties are brought into closer proximity with each other and FRET can occur. Ideally, minimal FRET efficiency is observed when the probe is unhybridized (for example, free in solution) and maximum FRET efficiency is observed when the hybridized DNA complex is completely formed. In an analgous manner, the second, third, and fourth target DNA strands (SEQ ID NO. 11, SEQ ID NO. 12, and SEQ ID NO. 13) may be expected to individually interact by Watson-Crick base-pairing with the nucleic acid probe (SEQ ID NO. 1) to form similar hybridized structures that each contain a single base-pairing mismatch.

General Experimental Conditions

The general experimental conditions were the same as those given above in Example 1, unless otherwise noted. All DNA used were High Purity Salt Free (HPSF®) DNA strands, produced and purified by MWG Biotech, Inc. (High Point, N.C., USA). For each hybridization and fluorescence experiment, samples of the DNA reagents were individually diluted to a final concentration of 0.4 micromoles per liter for each DNA reagent, or a final total DNA concentration of 0.8 micromoles per liter. As in example 1, the fluorescent behaviour of each of the four nucleic acid probe-target DNA mixtures was observed at room temperature and over a heating-cooling cycle.

Temperature-Dependentfluorescence Experiments

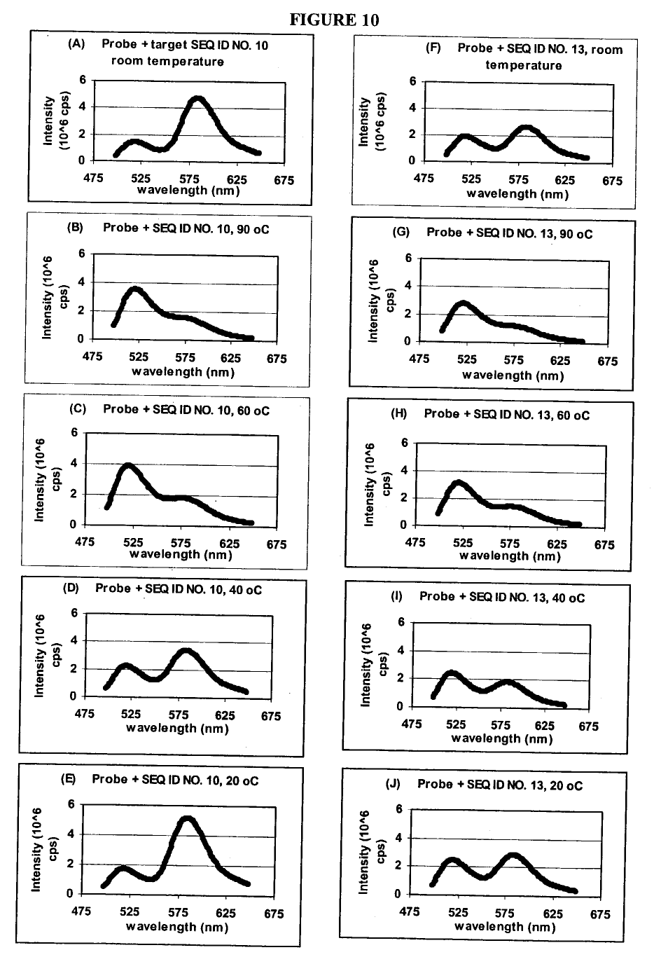

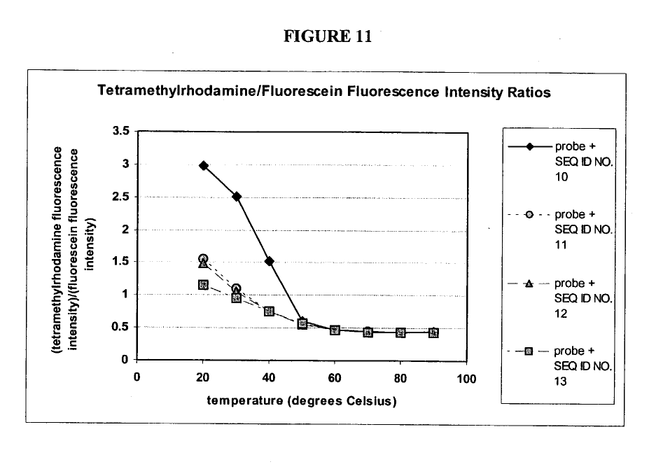

Representative fluorescence spectra of the nucleic acid probe (SEQ ID NO. 1) and the first target DNA strand (SEQ ID NO. 10), and of the nucleic acid probe (SEQ ID NO. 1) and the fourth target DNA strand (SEQ ID NO. 13), are shown in FIG. 10. The collected spectral data were used to calculate the ratio of tetramethylrhodamine intensity to fluorescein intensity. These ratios are given in Table 2 and depicted in FIG. 11.

TABLE 2
Tetramethylrhodamine/Fluorescein Fluorescence Intensity Ratio

| | |
|---|---|
| | Target DNA strand hybridized to |
| Temperature | nucleic acid probe (SEQ ID NO. 1) |

| (degrees | SEQ ID | SEQ ID | SEQ ID | SEQ ID |
|---|---|---|---|---|
| Celsius) | NO. 10 | NO. 11 | NO. 12 | NO. 13 |
| 20 | 2.977 | 1.55 | 1.488 | 1.163 |
| 30 | 2.516 | 1.098 | 1.049 | 0.962 |
| 40 | 1.517 | 0.756 | 0.748 | 0.754 |
| 50 | 0.608 | 0.574 | 0.561 | 0.573 |
| 60 | 0.475 | 0.475 | 0.464 | 0.469 |
| 70 | 0.444 | 0.444 | 0.438 | 0.442 |
| 80 | 0.442 | 0.437 | 0.434 | 0.434 |
| 90 | 0.444 | 0.44 | 0.441 | 0.438 |

At the initial room temperature scan, fluorescence emission of both the fluorescein (emission maximum at 522 nanometers) and of the tetramethylrhodamine (emission maximum at 580 nanometers) reporter moieties was observed for each of the nucleic acid probe-target DNA mixtures, indicating that at least some of the fluorescein reporter moieties were within FRET distance of at least some of the rhodamine reporter moieties. At room temperature, the ratio of tetramethylrhodamine intensity to fluorescein intensity was greater in the case of the perfectly base-paired nucleic acid probe (SEQ ID NO. 1) and the first target DNA strand (SEQ ID NO. 10), than in the case of the nucleic acid probe (SEQ ID NO. 1) and the second, third, or fourth target DNA strands (SEQ ID NO. 11, SEQ ID NO. 12, or SEQ ID NO. 13, respectively), where in each instance a single base-pairing mismatch would be present upon hybridization.

In each of the four combinations of nucleic acid probe and target DNA, upon heating the mixture to 90 degrees Celsius, the tetramethylrhodamine emission substantially decreased whereas the fluorescein emission substantially increased, indicating that in all four cases, FRET efficiency had decreased and that the relative distance between the two reporter moieties had increased. As each mixture was cooled to 20 degrees Celsius, the ratio of tetramethylrhodamine intensity to fluorescein intensity generally progressively increased, indicating an increase in FRET efficiency and a decrease in the relative distance between the two reporter moieties. However, a substantial difference in FRET behaviour was clearly observed as the mixtures were cooled from 40 degrees and lower. This is shown in FIG. 11, which depicts the temperature-dependent ratio of tetramethylrhodamine intensity to fluorescein intensity. In the case of the nucleic acid probe (SEQ ID NO. 1) and the first target DNA strand (SEQ ID NO. 10), perfect Watson-Crick base pairing is possible when the two DNA strands are fully hybridized. In the case of the nucleic acid probe (SEQ ID NO. 1) and the second, third, or fourth target DNA strands (SEQ ID NO. 11, SEQ ID NO. 12, or SEQ ID NO. 13, respectively), a single base-pairing mismatch is present in the base-pairing of the first recognition sequence (SEQ ID NO. 4) of the nucleic acid probe and each representative first site of a target allelic variant of an SNP (SEQ ID NO. 14, SEQ ID NO. 15, or SEQ ID NO. 16, respectively). In each of the latter three cases, a single base-pairing mismatch was observed to cause a surprisingly large decrease in FRET efficiency, relative to the case where there is no mismatch. From these observations, it can also be predicted that the melting temperature (Tₘ) is lower for cases where there is a single base-pairing mismatch between the first recognition sequence of the nucleic acid probe and a representative first site of a target allelic variant of an SNP, than when there is no mismatch. Thus, under a given set of hybridization conditions, the relative change in the spatial arrangement of the first reporter moiety (fluorescein) relative to the second reporter moiety (tetramethylrhodamine) is different when there is a single base-pairing mismatch between the nucleic acid probe and a representative target allelic variant of an SNP, than when there is no single base-pairing mismatch. Furthermore, under a given set of hybridization conditions, the relative change in a detectable signal (in this case, FRET), may be taken as an indicator of the presence or absence of a single base-pairing mismatch between the nucleic acid probe and a representative target allelic variant of an SNP.

In addition, at a given temperature below 40 degrees Celsius, FRET efficiency was lower in the case of the nucleic acid probe (SEQ ID NO. 1) and the fourth target DNA strand (SEQ ID NO. 13), than in the case of the nucleic acid probe and the second or third target DNA strands (SEQ ID NO. 14 or SEQ ID NO. 15, respectively). In other words, FRET efficiency was also observed to decrease as the position of the single base-pairing mismatch moved closer to the attachment site of the first reporter moiety (the fluorescein).

### Example 4

This example describes the use of a method sensitive to physical dimensions, atomic force microscopy, to detect the hybridization of a nucleic acid probe to a single molecule of a target DNA strand. Unless otherwise noted, all DNA sequences are given in the 5′ to 3′ direction.

Atomic Force Microscopy

Atomic force microscopy (AFM) makes use of an atomic force microscope, a scanning probe microscope, to study surface properties of materials from the atomic to the micron level. In this type of microscopy the sample surface is scanned in a rastering pattern. While scanning, the surface is probed with a tiny tip, about 2 micrometers long, which is attached to the free end of a cantilever, measuring between 100 and 200 micrometers long. Repulsive and attractive forces between the tip and the sample surface can cause the cantilever to bend or deflect. Several forces can cause cantilever deflection, although van der Waals forces provide the dominant interaction. During scanning, a laser spot is positioned on the reflective end of the cantilever. Light from the cantilever is directed by a mirror onto a split photo-diode divided into quadrants. By measuring the difference in signals between these quadrants as the cantilever bends, fluctuations of the cantilever position can be measured. Surface position is controlled through the use of piezoelectric scanners. The piezoelectric information, along with the cantilever deflection signal, are used to generate a topographical map of the sample surface.

The previous examples demonstrated that the nucleic acid probe binds to the first and to the second site of a target allelic variant of a SNP of interest and allow discrimination between allelic variants of an SNP. These examples used a method (fluorescence spectroscopy) that measured the average behavior of many molecules. Atomic force microscopy was investigated as a method to study a single molecule, representing a DNA molecule containing an SNP of interest.

Experimental Procedure

Ten-fold strength Tris-acetate-EDTA-magnesium buffer (10×TAE buffer with Mg²⁺) contains Tris base (400 millimoles per liter), glacial acetic acid (400 millimoles per liter), ethylenediaminetetraacetate (free acid) (10 millimole per liter), magnesium acetate (125 millimoles per liter), and sodium acetate (30 millimoles per liter). Single-strength Tris-acetate-EDTA-magnesium buffer (1×TAE buffer with Mg²⁺) is diluted from 10×TAE buffer with Mg²⁺, with pH adjusted to 7.8 with acetic acid.

Rolling circle amplification (RCA) (Liu et al. (1996), J. Am. Chem. Soc., 118:1587-1594) was used to prepare the target DNA strands. The DNA sequence GCTGCTGTCCGATGCGGTCACTGGTTAGTCCATGATGCACGGTAGCGCCGTTAGTCC AACTGGCATGTAGTATCGTCCGATGCAACCAGCGTCAG (SEQ ID NO. 17) was circularized and served as a template, using the primer TCGGACAGCAGCCTGACGCTGGTT (SEQ ID NO. 18) to begin the rolling circle amplification at its annealing site on the circularized template, according to the published method (Liu et al. (1996), J. Am. Chem. Soc., 118:1587-1594). The resulting RCA product consisted of long strands of DNA of varying lengths, each strand containing a variable number of repeating units of 95 nucleotides, joined end-to-end, of the target DNA sequence TCGGACAGCAGCCTGACGCTGGTTGCATCGGACGATACTACATGCCAGTTGGACTAA CGGCGCTACCGTGCATCATGGACTAACCAGTGACCGCA (SEQ ID NO. 19).

The nucleic acid probe (SEQ ID NO. 1) used in this experiment was identical to that used above in Examples 1, 2, and 3, and was manufactured by Integrated DNA Technologies, Inc (Coralville, Iowa, USA). The first recognition sequence (SEQ ID NO. 4) of this nucleic acid probe was complementary to a first target region consisting of the internal sequence ACGATACTACA (SEQ ID NO. 7) located at positions 32 through 42 of the repeating target DNA sequence (SEQ ID NO. 19). The second recognition sequence (SEQ ID NO. 5) of this nucleic acid probe was complementary to a second target region consisting of the internal sequence TGCCAGTTGG (SEQ ID NO. 8) located at positions 43 through 52 of the repeating target DNA sequence (SEQ ID NO. 19).

The crude RCA product was separated from the reaction mixture by ethanol precipitation, then resuspended in 100 microliters water. Hybridization to the nucleic acid probe (SEQ ID NO. 1) was carried out as follows: 2 microliters of the RCA product, 0.5 microliters nucleic acid probe stock solution (52.8 micromoles per liter, see Example 1), 1 microliters 10×TAE buffer with Mg²⁺, and 6.5 microliters of water were mixed and incubated at room temperature for 15-30 minutes.

All steps in sample preparation for AFM were performed in a humid chamber. A sample of the hybridization reaction mixture was applied to a freshly cleaved mica disk (VI mica from Structure Probe, Inc., West Chester, Pa., USA), approximately 1 centimeter in diameter, as follows: The freshly cleaved mica was pretreated for 1 minute with 3 microliters of 10×TAE buffer with Mg²⁺. The mica was rinsed three times with 100 microliters of distilled water, the water allowed to drain off, and the final rinse wicked away with a Kimwipe™ tissue. The hybridization reaction mixture was added as 2, 4, 6, and 8 microliter droplets, to achieve a variety of surface coverage densities, and allowed to rest on the mica for 3 minutes. A 2 microliter droplet of absolute ethanol was added to each hybridization reaction mixture droplet to precipitate the DNA onto the mica. The entire surface was rinsed immediately three times with 100 microliters of distilled water as described above. Finally, the mica was dried under a steady, light jet of argon gas.

The sample was imaged using a ThermoMicroscopes Explorer scan head and analyzed with the SPMLab software package also from ThermoMicroscopes (Sunnyvale, Calif., USA). Images were acquired in non-contact mode. A variety of set points (ranging from 30 to 70% of free oscillation amplitude) and feedback parameters were used in response to imaging conditions which changed over time.

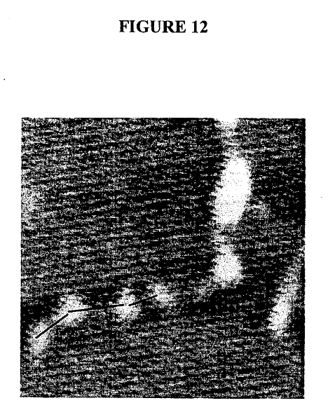

A representative, high magnification AFM micrograph is depicted in FIG. 12. Brighter portions of the image represent raised or elevated locations in the sample surface that are approximately 0.7 nanometers higher than the darkest features in the image. These bright image portions were reproducible, and were attributed to individual nucleic acid probe molecules, bound to a single-stranded long RCA strand that is not visible in the image. Measurement lines, connecting the centers of each bright image portion, were overlaid on the image (FIG. 12). The lengths of these line segments, reading from left to right, were 38, 56, and 28 nanometers, respectively. The distance between attachment sites of the nucleic acid probe to its repetitive target sequence was calculated to be 32 nanometers in a completely double stranded, linear, target DNA structure. However, the majority of the RCA product was expected to be single stranded, even taking into account the portions of the RCA product that are hybridized to the nucleic acid probe, and thus the binding locations were not believed to be necessarily separated by this calculated 32 nanometer repeat distance. The three values for separation obtained in this experiment (38, 56 and 28 nanometers) were interpreted to represent one repeat distance, two repeat distances (where one set of binding sites was “skipped”, or not bound, by a nucleic acid probe molecule), and one repeat distance.

The nucleic acid probe in this example can be labelled with one or more fluorescent molecules, which can be one of the reporter moieties, both reporter moieties (for example, a FRET pair), or an independent label or labels. An AFM sample consisting of such a fluorescently labelled nucleic acid probe, hybridized to its RCA target strand, can be illuminated with light at an appropriate excitation wavelength, and the resulting emission detected. The resulting bright locations imaged by AFM are thus unambiguously identified as individual nucleic acid probe molecules, each bound to a pair of target regions in the RCA strand.

These results demonstrate that a single molecule of a nucleic acid probe of the invention, hybridized to its target, can be observed. Thus the limit of detection of a target (such as a single nucleotide polymorphism), using a nucleic acid probe of the invention, is a single molecule. Hybridization of the nucleic acid probe to its target results in a detectable signal, such as a FRET signal (where the probe includes a FRET pair as the first and second reporter moieties), or a structural or configurational change in the nucleic acid probe that is detectable by methods sensitive to physical dimensions, such as an elevated structure detected by AFM (where unmodified bases in the first and second recognition sequences of the probe may be considered to be the first and second reporter moieties). Thus, in this experiment, the RCA product represented a target molecule (analogous to a DNA molecule with a single nucleotide polymorphism or SNP) containing a first and second target region (analogous to a first and second site of a target allelic variant of a single nucleotide polymorphism) that hybridize, respectively, to a first and second recognition sequence of a nucleic acid probe of the present invention; the detectable signal was a structural or configurational change in the nucleic acid probe observed as a bright or elevated structure detected by AFM. The RCA product, or a similarly constructed, long molecule containing repeating nucleic acid or nucleic acid mimic sections, could alternatively serve as an accessory molecule of the invention. In this case, a single, long accessory molecule could either tether a multiplicity of nucleic acid probe molecules of the same type (each capable of binding the same set of target regions, such as a first and second site of a target allelic variant of an SNP) or the single accessory molecule could tether a multiplicity of nucleic acid probe molecules, each capable of binding to a different target molecule (that is to say, capable of binding to a different set of first and second target regions, such as different first and second sites of target allelic variants of an SNP).

### Example 5

The following describes examples of different systems that employ the methods and probes of the present invention, and examples of applications of these systems.

Two-Strand Systems

These systems employ the first method of detecting a single nucleotide polymorphism (SNP) as described in the Detailed Description of the Invention. The nucleic acid probe may exist in any of a continuum of configurations or structures, with one extreme being an open or generally linear configuration that is not base-paired to the target allelic variant of the SNP of interest, and the opposite extreme being a closed, circular or looped configuration, where the first and the second recognition sites of the nucleic acid probe are base-paired to the first and the second sites, respectively, of the target allelic variant of the SNP, and where the linking element of the nucleic acid probe is not base-paired to the target allelic variant of the SNP and thus forms the “open” portion of the circular or looped structure (FIGS. 1 and 2). The configuration of structure assumed by the nucleic acid probe when fully hybridized to the target allelic variant of the SNP results in a change in the spatial arrangement of the first reporter moiety relative to the second reporter moiety, and thus changes the detectable signal that is a result of the interaction of the two reporter moieties.

Hybridization between the nucleic acid probe and the SNP is differential, that is to say, the base-pairing between the nucleic acid probe and the SNP (and thus the resulting change in detectable signal) is different when there is no single-base mismatch between the nucleic acid probe and the allelic variant of the SNP than when there is a single-base mismatch. This differential hybridization may be displayed in the form of a difference in hybridization binding strength for the case where there is no single-base mismatch between the nucleic acid probe and the allelic variant of the SNP than when there is a single-base mismatch. Thus, for example, at a given temperature (preferably near or below the annealing temperature or melting temperature of the hybridized structure with no single-base mismatch between the nucleic acid probe and the allelic variant of the SNP), when all other hybridization conditions are the same, the hybridization binding strength is greater for the more perfectly base-paired structure. By “annealing temperature” is meant the temperature (usually determined during cooling of a system) at which half of the strands of a complementary pair of nucleic acid strands are paired. By “melting temperature” is meant the temperature (usually determined during heating of a system) at which half of the strands of a complementary pair of nucleic acid strands are unpaired. An assay system can therefore be poised at a given temperature, or the temperature of the system can be increased or decreased, and the behavior of the detectable signal produced by the system can be compared to equivalent experiments for systems where there is no single-base mismatch between the nucleic acid probe and the allelic variant of the SNP and where there is a known single-base mismatch. For example, it would be expected that the hybridization binding strength is greater for the more perfectly base-paired structure at higher temperatures, where the strands of a structure with a single-base mismatch would be less perfectly base-paired and have weaker interactions between the strands, possibly allowing the strands to dissociate. In such a case, if a detectable signal is observable only when the nucleic acid probe is hybridized to the SNP, then the detectable signal would be observable at higher temperatures for the more perfectly base-paired structure (such as where there is no single-base mismatch between the nucleic acid probe and the allelic variant of the SNP) than for a less perfectly base-paired structure (such as where there is a single-base mismatch between the nucleic acid probe and the allelic variant of the SNP). In other cases, a detectable signal may be observable for and characteristic of the unhybridized nucleic acid probe, and this detectable signal changes upon hybridization. In such cases, it is possible to estimate or quantitate the amounts of unhybridized and hybridized nucleic acid probe present in a system by observation or measurement of the detectable signals that are characteristic, respectively, of the unhybridized and hybridized nucleic acid probe. Systems can be designed to be specific for one or more specific target allelic variants of a given SNP. Assays and kits using the methods of the invention may be designed to include positive or negative controls (for example, to verify that the reaction components of the method function as intended), and may further include washing steps (for example, to decrease background noise). Three-Strand Systems

These systems employ the second and third methods of detecting a single nucleotide polymorphism (SNP) as described in the Detailed Description of the Invention. In the systems employing the second method, both the first and the second reporter moieties are located on the nucleic acid probe. In the systems employing the third method, the first reporter moiety is located on the nucleic acid probe and the second reporter moiety is located on the accessory molecule. In systems employing either the second or the third method, the nucleic acid probe may again exist in any of a continuum of configurations or structures from an open or generally linear configuration that is not base-paired to the target allelic variant of the SNP of interest, and the opposite extreme being a closed, circular or looped configuration, where the first and the second recognition sites of the nucleic acid probe are base-paired to the first and the second sites, respectively, of the target allelic variant of the SNP. The hybridization behaviour of the nucleic acid probe to the SNP is similar to that when using the first method of detecting an SNP, but systems employing either the second or the third method additionally use an accessory molecule. The linking element of the nucleic acid probe may be capable of complementary base-pairing with a sequence of the accessory molecule.

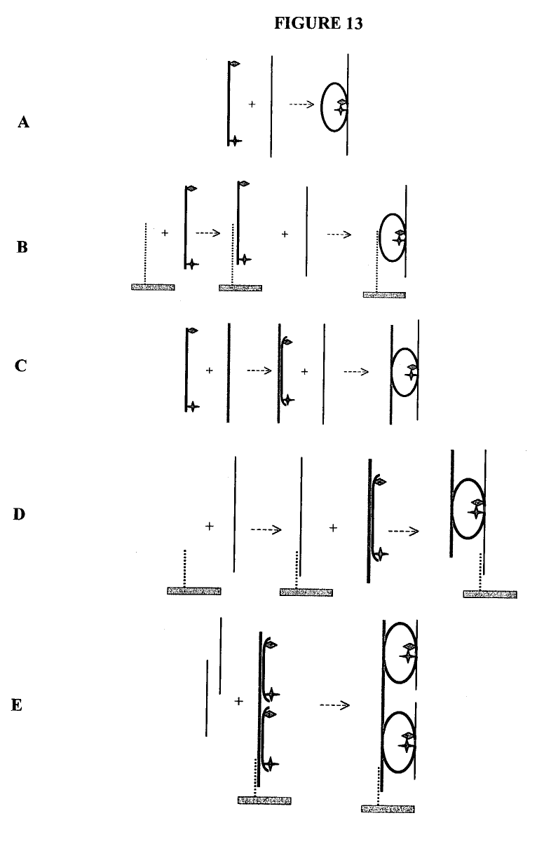

The accessory molecule can serve one or more functions. For example, the accessory molecule can limit the freedom of the nucleic acid probe to acquire configurations that result in false positive signals, a potential weakness of a two-strand system where under certain conditions (such as at sufficiently low temperatures), it may be possible for a false positive signal to result from interstrand or intrastrand interactions of the nucleic acid probe alone. Another potential function is for the accessory molecule to limit the range of internal motions of the nucleic acid probe, thus improving or enhancing its ability to hybridize correctly to the intended target (i.e., the target allelic variant of the SNP). Another potential function is for the accessory molecule to limit the range of locations on the intended target (for example, a strand of DNA that contains the target allelic variant of the SNP) with which the nucleic acid probe can interact, thus improving or enhancing the stringency of the hybridization. Yet another potential function is for the accessory molecule to serve as a mechanism to bind the nucleic acid probe, directly or indirectly, to a specific location, such as to a solid surface. For example, the accessory molecule can bind the nucleic acid probe (and thus the SNP, when the SNP is hybridized to the nucleic acid probe), to a molecular structure or to the surface of microbeads, magnetic particles, a microarray, or the surfaces of a chamber. Depictions of these three-strand systems are shown in FIG. 13.

As in the case for the two-strand systems, in a three-strand system, if a detectable signal is observable only when the nucleic acid probe/accessory molecule complex is hybridized to the SNP, then the detectable signal would be observable at higher temperatures for the more perfectly base-paired structure (such as where there is no single-base mismatch between the nucleic acid probe and the allelic variant of the SNP) than for a less perfectly base-paired structure (such as where there is a single-base mismatch between the nucleic acid probe and the allelic variant of the SNP). In other cases, a detectable signal may be observable for and characteristic of the unhybridized nucleic acid probe/accessory molecule complex, and this detectable signal changes upon hybridization. In such cases, it is possible to estimate or quantitate the amounts of unhybridized and hybridized nucleic acid probe/accessory molecule complex present in a system by observation or measurement of the detectable signals that are characteristic, respectively, of the unhybridized and hybridized nucleic acid probe/accessory molecule complex. Systems can be designed to be specific for one or more specific target allelic variants of a given SNP. Assays and kits using the methods of the invention may be designed to include positive or negative controls (for example, to verify that the reaction components of the method function as intended), and may further include washing steps (for example, to decrease background noise).

Applications

The different systems employing methods and probes of the present invention may be applied to various assay formats. Non-limiting examples of these are given below, where for purposes of illustration the two reporter moieties are members of a FRET pair, located on the nucleic acid probe.

1. A two-strand assay performed with all components in solution phase. The sample that may contain an SNP of interest is contacted with the nucleic acid probe. Under appropriate hybridization conditions, the nucleic acid probe hybridizes to the SNP and the resulting signal is detected. See FIG. 13A.

2. A two-strand assay performed with at least one component on a solid substrate. In one possible assay, the sample suspected of containing the SNP of interest is bound, directly or indirectly, to a solid substrate. For example, a capture DNA strand is affixed to the surface of a solid substrate (for example, microbeads, magnetic particles, surfaces of a microtiter well, a flow-through chamber, or a microarray chip). The SNP in solution contacts the capture DNA strand and is bound, thus immobilizing the SNP onto the solid substrate. The nucleic acid probe is contacted with the SNP/capture DNA strand complex, and under appropriate hybridization conditions, the nucleic acid probe hybridizes to the SNP and the resulting signal is detected. Alternatively, the nucleic acid probe is affixed, directly or indirectly, to the surface of a solid substrate, and the SNP in solution is allowed to contact and hybridize to the nucleic acid probe. See FIG. 13B. Preferably, the detectable signal is proportional to the concentration of the target allelic variant of the SNP that matches the nucleic acid probe.

2. A three-strand assay performed with all components in solution phase. One ore more nucleic acid probes is contacted with the accessory molecule, and the linking element of the nucleic acid probe base-pairs with a sequence of the accessory molecule. The resulting two-strand “capture device” (the nucleic acid probe/accessory molecule complex) is contacted with the sample containing an SNP of interest. Under appropriate hybridization conditions, each nucleic acid probe hybridizes to its target allelic variant of the SNP and the resulting signal is detected. See FIG. 13C. A suitable signal could also be generated in a parallel case where the first reporter moiety is located on the nucleic acid probe and the second reporter moiety is located on the accessory molecule.

3. Three-strand assays performed on a solid substrate. Immobilizing one or more components on a solid substrate may be advantageous, for example, in permitting lower concentrations of reagents to be used, in permitting the re-use of reagents (such as the accessory molecule, the nucleic acid probe, or both), and in localizing an assay in a discrete area and thus allowing multiple assays to be run in a small area (such as in an array).

In one possible assay, a capture DNA strand is affixed to the surface of a solid substrate (for example, microbeads, magnetic particles, surfaces of a microtiter well, a flow-through chamber, or a microarray chip). The SNP in solution contacts the capture DNA strand and allowed to bind, thus immobilizing the SNP indirectly onto the solid substrate. A complex including the nucleic acid probe hybridized to an accessory molecule is contacted with the SNP/capture DNA strand complex, and under appropriate hybridization conditions, the nucleic acid probe/accessory molecule complex hybridizes to the SNP and the resulting signal detected. See FIG. 13D. Preferably, the detectable signal is proportional to the concentration of the target allelic variant of the SNP that matches the nucleic acid probe.

In another possible assay, the accessory molecule is affixed to the surface of a solid substrate, either directly or indirectly (for example, through binding to an intermediate molecule). The nucleic acid probe is contacted with the immobilized accessory molecule, and the linking element of the nucleic acid probe base-pairs with a sequence of the accessory molecule. The resulting immobilized two-strand “capture device” (the nucleic acid probe/accessory molecule complex) is contacted with the sample containing an SNP of interest. Under appropriate hybridization conditions, the nucleic acid probe hybridizes to the SNP and the resulting signal detected. A suitable signal could also be generated in a parallel case where the first reporter moiety is located on the nucleic acid probe and the second reporter moiety is located on the accessory molecule.

4. Combinations. A variety of alternative combinations of nucleic acid probe, accessory molecule, and sample are envisioned. In the simplest combinations, a single nucleic acid probe (with or without a single accessory molecule) is used to detect the presence of a single target allelic variant of an SNP of interest. For example, a single nucleic acid probe, designed to hybridize with no mismatch to a single particular target allelic variant of an SNP, is contacted and incubated under a given set of hybridization conditions with a sample suspected of containing the SNP, and the resulting detectable signal may indicate the presence or absence of that single target allelic variant of an SNP, for example as the presence of a perfect match between the probe and an SNP present in the sample, or as the absence of a perfect match (which may be a single base-pairing mismatch in an SNP present in the sample) between the probe and the sample. In other alternatives, two or more nucleic acid probes may be used to analyze a sample for one or more target allelic variants of an SNP of interest. Multiple probes (of one type or of more than one type) on a single accessory molecule may be used to analyze a sample for one or more target allelic variants of an SNP of interest. See FIG. 13E. In assays using the third method of the invention, a single type or multiple types of nucleic acid probe may be combined with different accessory molecules in analyzing a sample for one or more target allelic variants of an SNP of interest.

### Examples

I. An example of a two-strand assay distinguishing between two possible allelic variants of an SNP (and the three possible genotypes for this SNP) follows.

The objective of the following assay is to detect in an individual patient the presence of a wild type or mutant SNP at the locus associated with the majority of human Hereditary Hemochromatosis (HH) patients in the United States. There are three possible cases: a) homozygous wild type SNP at this locus on each of the DNA strands representing the gene; b) heterozygous mutant SNP, that is to say, a mutant SNP on one of the two strands of DNA representing the gene; and c) homozygous mutant SNP, that is to say, a mutant SNP on both of the two strands of DNA representing the gene. The majority of the population, which does not harbor or transmit this genetic disease, have the “normal” or homozygous wild type genotype. Individuals who have the heterozygous mutant SNP genotype do not usually develop symptoms of this disease, but are considered carriers who can pass the disease to their children. Patients who have the homozygous mutant SNP genotype do not always display symptoms of the disease because it is a treatable disease, or because they lose iron often (particularly through bleeding, such as, in women, during menstruation) and show minimal effects from the disease; however these patients should be made aware of the potential risk for death from the disease.

A buccal swab DNA sample is taken from an individual to be screened for human Hereditary Hemochromatosis (HH). The portion of the DNA containing the SNP of interest is amplified, for example, by symmetrical PCR amplification (to give equal quantities of each of the amplified complementary strands), or, preferably, by asymmetrical PCR (to give larger quantities of the target strand intended for hybridization in the assay, than of its complement). In some cases, unamplified DNA may be used. If necessary, the sample may be further processed as described above under the subheading “Sample”, under the heading “A first method for detecting a single nucleotide polymorphism”.

The assay system includes an interrogation means, a solid substrate, at least one FRET-based nucleic acid probe, and a detection means. Where one nucleic acid probe is used in this assay, the single probe preferably is capable of differentially binding to, and thus discriminating between, the wild type and mutant allelic variants of the HH SNP. Where two nucleic acid probes are used in this assay, the first probe is designed to include a first recognition sequence that is complementary to a first site of the wild type allelic variant of the HH SNP and to provide the greatest FRET signal for the wild type HH SNP, and the second probe is designed to include a first recognition sequence that is complementary to a first site of the mutant allelic variant of the HH SNP and to provide the greatest FRET signal for the mutant HH SNP.

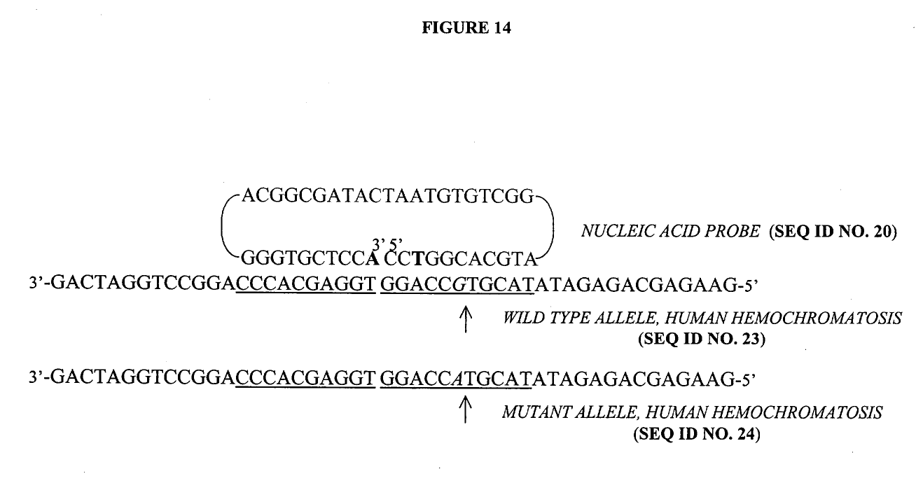

In a specific example, a single nucleic acid probe, designed to perfectly complement the wild type allelic variant of the HH SNP, and target DNA strands that represented models of the wild type and mutant allelic variants of the HH SNP, were designed (FIG. 14). All DNA used in these experiments was synthesized by Integrated DNA Technologies (Coralville, Iowa, USA).

The nucleic acid probe contained 42 nucleotides and had the sequence CCTGGCACGTAGGCTGTGTAATCATAGCGGCAGGGTGCTCCA (SEQ ID NO. 20) and contained (a) a first recognition sequence made up of the 11 terminal nucleotides at the 5′ terminus with the sequence CCTGGCACGTA (SEQ ID NO. 21); (b) a second recognition sequence made up of the 10 terminal nucleotides at the 3′ terminus with the sequence GGGTGCTCCA (SEQ ID NO. 22); (c) a linking element made up of the intervening 21 nucleotides with the sequence GGCTGTGTAATCATAGCGGCA (SEQ ID NO. 6); (d) a first reporter moiety (a fluorescein molecule attached to the thymine located 3 nucleotides from the 5′ terminus of SEQ ID NO. 20); and (e) a second reporter moiety (a tetramethylrhodamine molecule, attached to the 3′ terminal adenosine of SEQ ID NO. 20).

A model of the wild type allelic variant of the human Hereditary Hemochromatosis SNP consisted of 48 nucleotides and had the sequence GAAGAGCAGAGATATACGTGCCAGGTGGAGCACCCAGGCCTGGATCAG (SEQ ID NO. 23). A model of the mutant allelic variant of the human Hereditary Hemochromatosis SNP consisted of the 48 nucleotides and had the sequence GAAGAGCAGAGATATACGTACCAGGTGGAGCACCCAGGCCTGGATCAG (SEQ ID NO. 24). These two sequences are identical except for the single polymorphic locus at position 20, which is a guanine in the wild type HH SNP and is an adenine in the mutant HH SNP.

The first recognition sequence (SEQ ID NO. 21) of the nucleic acid probe (SEQ ID NO. 20) was complementary to a first site of the wild type allelic variant of the HH SNP that consisted of the internal sequence TACGTGCCAGG (SEQ ID NO. 25) located at positions 15 through 25 of SEQ ID NO. 23 and contained the polymorphic locus at position 20. The second recognition sequence (SEQ ID NO. 22) of this nucleic acid probe was complementary to a second site of the wild type allelic variant of the HH SNP that consisted of the internal sequence TGGAGCACCC (SEQ ID NO. 26) located at positions 31 through 40 of SEQ ID NO. 23. In this example, under a given set of hybridization conditions, the single nucleic acid probe (SEQ ID NO. 20) hybridizes more perfectly with its exact complement (the wild type allelic variant of the HH SNP) than with a sequence containing a single base-pairing mismatch (the mutant allelic variant of the HH SNP). Thus, the measured FRET signal, caused by the relative change in the spatial arrangement of the first reporter moiety (fluorescein) relative to the second reporter moiety (tetramethylrhodamine), is larger when the probe hybridizes to the wild type allelic variant of the HH SNP than when the probe hybridizes to the mutant allelic variant of the HH SNP. As had also been seen in Example 3 above, a single base-pairing mismatch causes a surprisingly large decrease in FRET efficiency, relative to the case where there is no mismatch. Therefore, under a given set of hybridization conditions, the relative change in a detectable signal (in this case, FRET), may be taken as an indicator of the presence or absence of a single base-pairing mismatch between the nucleic acid probe and a representative target allelic variant of an SNP.

In the simplest case where two nucleic acid probes are used, the nature and location of the first and second reporter moieties that form the members of the FRET pair are identical in each of the two probes. Alternatively, as in the approach described below (under the subheading “Dual nucleic acid probes, single DNA sample”), the nature or the location or both of the first and second reporter moieties that form the members of the FRET pair may be different in the nucleic acid probe specific for the wild type SNP than in the nucleic acid probe specific for the mutant SNP. In either case, for each nucleic acid probe, the nature and location of the first and second reporter moieties that form the members of the FRET pair are preferably selected to maximize the difference between the signals obtained when the probe is hybridized and when the probe is not hybridized. Also preferably, in the approach described below (under the subheading “Single nucleic acid probe, single DNA sample”), where a single nucleic acid probe is used to distinguish between the wild type and the mutant SNP, the nature and location of the first and second reporter moieties that form the members of the FRET pair are selected to maximize the difference between the signals obtained when the probe is hybridized to the wild type SNP and when the probe is hybridized to the mutant SNP. The two probes each also include a second recognition sequence that is complementary to a second site of the target allelic variant of the Human Hereditary Hemochromatosis SNP, wherein this second site can be identical in the wild type and the mutant SNP. The two probes each also include a linking element, and a first and a second reporter moiety, identical in the two probes. The interrogation means includes a blue-light emitting diode or similar means of exciting the donor member of the FRET pair. The detection means includes a sensor such as at least one light sensing photodiode that is sensitive to a wavelength or wavelenghts emitted by the FRET pair, and is optionally equipped with appropriate filters. The whole assay system may be integrated into a microplate reader format or other high throughput format.

The assay includes a solid substrate (preferably glass or other silica-based material, or a polymeric plastic), such as a slide or chip or a microplate well. Preferably, the solid substrate is designed so that the sample containing the SNP of interest is localized to a small discrete area or areas on the solid substrate, in order to concentrate the detectable signal in that area. Multiple copies of a capture DNA (or PNA) strand are affixed to a small, discrete area on the solid substrate (such as a round spot on a slide or chip, or on the bottom or other surfaces of microplate wells) to the solid substrate. The capture DNA (or PNA) strand includes a DNA sequence that is complementary to a region of the amplified DNA sample, and that is other than the first site and the second site of the SNP intended for hybridization by the nucleic acid probe. Preferably, the solid substrate is treated to bind minimal or no DNA (or PNA) in areas other than those discrete areas wherein the capture DNA (or PNA) strands are immobilized.

The sample containing the DNA is contacted with the solid substrate, then contacted with the nucleic acid probe, allowed to hybridize, and the detectable signal observed. Non-limiting examples of different approaches for testing a sample immobilized on the solid substrate follow.

A. Single nucleic acid probe, single DNA sample. The immobilized sample is contacted with the nucleic acid probe with a first recognition sequence that is complementary to a first site of the wild type allelic variant of the human Hereditary Hemochromatosis SNP. The observed detectable signal under appropriate hybridization conditions is compared to the known values of the signal observed for the same nucleic acid probe when hybridized to reference samples of the homozygous wild type, heterozygous mutant, and homozygous mutant SNP. When hybridized to the nucleic acid probe that is specific for the wild type SNP, the homozygous wild type reference sample gives a high FRET signal, the heterozygous mutant reference sample gives an intermediate FRET signal, and the homozygous mutant reference sample gives a low or no FRET signal. Alternatively, this type of assay could be designed to be specific for the mutant SNP. This type of assay preferably includes normalization of the observed FRET signal to account for the amount of sample DNA immobilized to the solid substrate, and preferably also includes accurate controls to verify that each of the different components of the system function as designed.

B. Dual nucleic acid probes, duplicate DNA samples. Two nucleic acid probes are used in this approach for a differential assay. Two identical solid substrates are prepared and identically contacted with the sample DNA solution. The first immobilized sample DNA is contacted with the nucleic acid probe specific for the wild type SNP, and the second immobilized sample DNA is contacted with the nucleic acid probe specific for the mutant SNP. The observed detectable signal under appropriate hybridization conditions is compared to the known values of the signal observed from the two types of probe hybridized individually to reference samples of the homozygous wild type, heterozygous mutant, and homozygous mutant HH SNP. The nucleic acid probe specific for the wild type SNP gives a maximal FRET signal when hybridized to the homozygous wild type SNP, an intermediate FRET signal when hybridized to the heterozygous mutant SNP, and a minimal FRET signal when hybridized to the homozygous mutant SNP. The nucleic acid probe specific for the mutant SNP gives a minimal FRET signal when hybridized to the homozygous wild type SNP, an intermediate FRET signal when hybridized to the heterozygous mutant SNP, and a maximal FRET signal when hybridized to the homozygous mutant SNP. These reference values are used in the evaluation of the two signals obtained for each sample DNA. Optimal hybridization conditions and designs of the two nucleic acid probes can be determined by theoretical design and by experimentation, in order to determine the relative magnitude of the observed signal for an individual DNA sample.

C. Dual nucleic acid probes, single DNA sample. In this approach, two nucleic acid probes are used. The nature or the location or both of the first and second reporter moieties that form the members of the FRET pair are different in the nucleic acid probe specific for the wild type SNP than in the nucleic acid probe specific for the mutant SNP. Thus, the FRET signal from the probe specific for the wild type SNP is different (for example, has a different donor emission maximum wavelength) from that from the probe specific for the mutant SNP. The two probes are contacted simultaneously with a single immobilized DNA sample under hybridization conditions that preferably result in maximal binding of each probe to its perfect complement (that is to say, either the wild type or the mutant SNP); under such conditions, hybridization of the wild type probe to the wild type SNP results in a FRET signal that is much greater than that produced by hybridization of the wild type probe to the mutant SNP, and hybridization of the mutant probe to the mutant SNP results in a FRET signal that is much greater than that produced by hybridization of the mutant probe to the wild type SNP. The intensity and type of signal is used to distinguish between the three possible genotypes of this SNP. In a DNA sample containing the homozygous wild type SNP, the observed signal is primarily caused by the FRET pair of the wild type probe. In a DNA sample containing the homozygous mutant SNP, the observed signal is primarily caused by the FRET pair of the mutant probe. In a DNA sample containing the heterozygous mutant SNP, the observed signal is a combination of FRET signals from the wild type probe and the mutant probe.

II. An example of a three-strand assay distinguishing between two possible allelic variants of an SNP (and the three possible genotypes for this SNP) follows.

The objective of the following assay is to detect in an individual patient the presence of a wild type or mutant SNP at the locus associated with the majority of human Hereditary Hemochromatosis (HH) patients in the United States. There are three possible cases: a) homozygous wild type SNP at this locus on each of the DNA strands representing the gene; b) heterozygous mutant SNP, that is to say, a mutant SNP on one of the two strands of DNA representing the gene; and c) homozygous mutant SNP, that is to say, a mutant SNP on both of the two strands of DNA representing the gene. The majority of the population, which does not harbor or transmit this genetic disease, have the “normal” or homozygous wild type genotype. Individuals who have the heterozygous mutant SNP genotype do not usually develop symptoms of this disease, but are considered carriers who can pass the disease to their children. Patients who have the homozygous mutant SNP genotype do not always display symptoms of the disease because it is a treatable disease, or because they lose iron often (particularly through bleeding, such as, in women, during menstruation) and show minimal effects from the disease; however these patients should be made aware of the potential risk for death from the disease.

A buccal swab DNA sample is taken from an individual to be screened for human Hereditary Hemochromatosis (HH). The portion of the DNA containing the SNP of interest is amplified, for example, by symmetrical PCR amplification (to give equal quantities of each of the amplified complementary strands), or, preferably, by asymmetrical PCR (to give larger quantities of the target strand intended for hybridization in the assay, than of its complement). In some cases, unamplified DNA may be used. If necessary, the sample may be further processed as described above under the subheading “Sample”, under the heading “A first method for detecting a single nucleotide polymorphism”.

The assay system includes an interrogation means, a solid substrate, at least one FRET-based nucleic acid probe (of one or more types), at least one accessory molecule, and a detection means. Two nucleic acid probes may be used in this assay, wherein the first probe is designed to include a first recognition sequence that is complementary to a first site of the wild type allelic variant of the HH SNP and to provide the greatest FRET signal for the wild type HH SNP, and the second probe is designed to include a first recognition sequence that is complementary to a first site of the mutant allelic variant of the HH SNP and to provide the greatest FRET signal for the mutant HH SNP. In the simplest case, for each nucleic acid probe, the nature and location of the first and second reporter moieties that form the members of the FRET pair are identical. Alternatively, as in the approach described below (under the subheading “Differentially labelled, dual nucleic acid probe/accessory molecule complexes, duplicate or identical DNA samples”), the nature or the location or both of the first and second reporter moieties that form the members of the FRET pair may be different in the nucleic acid probe specific for the wild type SNP than in the nucleic acid probe specific for the mutant SNP. In either case, for each nucleic acid probe, the nature and location of the first and second reporter moieties that form the members of the FRET pair are preferably selected to maximize the difference between the signals obtained when the probe is hybridized and when the probe is not hybridized. Also preferably, in the approach described below (under the subheading “Single nucleic acid probe/accessory molecule complex, single DNA sample”), where a single nucleic acid probe is used to distinguish between the wild type and the mutant SNP, the nature and location of the first and second reporter moieties that form the members of the FRET pair are selected to maximize the difference between the signals obtained when the probe is hybridized to the wild type SNP and when the probe is hybridized to the mutant SNP. The two probes each also include a second recognition sequence that is complementary to a second site of the target allelic variant of the HH SNP, wherein this second site can be identical in the wild type and the mutant SNP. The two probes each also include a linking element, and a first and a second reporter moiety, identical in the two probes. The accessory molecule is designed to interact with the nucleic acid probe or probes in order to serve at least one of the functions of an accessory molecule such as described above under the subheading “Accessory molecule”, under the heading “A second method for detecting a single nucleotide polymorphism”. The interrogation means includes a blue-light emitting diode or similar means of exciting the donor member of the FRET pair. The detection means includes a sensor such as at least one light sensing photodiode that is sensitive to a wavelength or wavelenghts emitted by the FRET pair, and is optionally equipped with appropriate filters. The whole assay system may be integrated into a microplate reader format or other high throughput format.

The assay includes a solid substrate (preferably glass or other silica-based material, or a polymeric plastic), such as a slide or chip or a microplate well. Preferably, the solid substrate is designed so that the nucleic acid probe/accessory molecule complex is localized to a small discrete area or areas on the solid substrate, such as a spot or spots on a slide or chip, in order to concentrate the detectable signal in that area.

The nucleic acid probe/accessory molecule complex may be immobilized to the solid substrate in different ways. For example, multiple copies of a capture DNA (or PNA) strand are affixed to a small, discrete area on the solid substrate (such as a round spot on a slide or chip, or on the bottom or other surfaces of microplate wells) to the solid substrate. Preferably, the solid substrate is treated to bind minimal or no DNA (or PNA) in areas other than those discrete areas wherein the capture DNA (or PNA) strands are immobilized. The capture DNA (or PNA) strand can include a DNA (or PNA) sequence that is complementary to a sequence of the accessory molecule that is not complementary to the linking element of the nucleic acid probe, leaving that sequence of the accessory molecule that is complementary to the linking element available to bind the probe. In this case, the length of the portion of the accessory molecule necessary to extend the nucleic acid probe away from the surface of the solid subtrate is relatively minimized, and this procedure may be preferred for economic or kinetic reasons. In one alternative, the accessory molecule may include a nucleic acid sequence or a nucleic acid mimic (such as a peptide nucleic acid) sequence which is complementary to the linking element of the nucleic acid probe, and thus the accessory molecule can serve the purpose of the capture DNA (or PNA) strand. Preferably, the result of affixing the nucleic acid probe/accessory molecule complex to the solid surface is a nucleic acid probe that provides a maximal differential signal upon hybridization, under conditions constrained partially by the accessory molecule, to the SNP of interest, wherein the signal differentiates between the possible allelic variants of that SNP. In some cases, it may be desirable to determine the relative concentration of the nucleic acid probe/accessory molecule complex associated with each spot prior to contact with the DNA sample, for example, by interrogating one or both reporter moieties and detecting the resulting signal or signals due to the individual reporter moiety. Such a measurement allows the final result to be normalized to the actual number of immobilized probe molecules, and can account for variance in the actual number of nucleic acid probe molecules in a spot (resulting from manufacturing irregularities).

The sample containing the DNA is contacted with the nucleic acid probe/accessory molecule complex affixed to the solid substrate, allowed to hybridize, and the detectable signal observed. Non-limiting examples of different approaches for testing a sample immobilized on the solid substrate follow.

A. Single nucleic acid probe/accessory molecule complex, single DNA sample. The sample is contacted with the immobilized nucleic acid probe/accessory molecule complex; the nucleic acid probe used contains a first recognition sequence that is complementary to a first site of the wild type allelic variant of the HH SNP. The observed detectable signal under appropriate hybridization conditions is compared to the known values of the signal observed for the same nucleic acid probe when hybridized to reference samples of the homozygous wild type, heterozygous mutant, and homozygous mutant HH SNP. When hybridized to the nucleic acid probe that is specific for the wild type SNP, the homozygous wild type reference sample gives a high FRET signal, the heterozygous mutant reference sample gives an intermediate FRET signal, and the homozygous mutant reference sample gives a low or no FRET signal. Alternatively, this type of assay could be designed to be specific for the mutant SNP. This type of assay preferably includes normalization of the observed FRET signal to account for the amount of sample DNA immobilized to the solid substrate, and preferably also includes accurate controls to verify that each of the different components of the system function as designed.

B. Dual nucleic acid probe/accessory molecule complexes, duplicate or identical DNA samples. Two nucleic acid probes are used in this approach for a differential assay, one probe being specific for the wild type SNP and the other probe being specific for the mutant SNP. Each probe is individually complexed with the accessory molecule and each complex separately immobilized on the solid substrate. Preferably, the two nucleic acid probe/accessory molecule complexes are immobilized individually on the same solid substrate (for example, as adjacent but separate spots on a slide or chip), allowing the two complexes to be exposed to the DNA sample under essentially identical conditions. Each immobilized nucleic acid probe/accessory molecule complex is then contacted, separately or simultaneously, with duplicate DNA samples (for example, with duplicate aliquots of a solution containing the DNA sample) or with an identical DNA sample (for example, the entire chip or slide, bearing individual spots of each immobilized nucleic acid probe/accessory molecule complex, is exposed to a single aliquot of a solution containing the DNA sample). The observed detectable signal from each of the two nucleic acid probe/accessory molecule complexes under appropriate hybridization conditions is compared to known values of the signal observed from the two types of nucleic acid probe/accessory molecule complexes hybridized individually to reference samples of the homozygous wild type, heterozygous mutant, and homozygous mutant SNP. The nucleic acid probe/accessory molecule complex specific for the wild type SNP gives a maximal FRET signal when hybridized to the homozygous wild type SNP, an intermediate FRET signal when hybridized to the heterozygous mutant SNP, and a minimal FRET signal when hybridized to the homozygous mutant SNP. The nucleic acid probe/accessory molecule complex specific for the mutant SNP gives a minimal FRET signal when hybridized to the homozygous wild type SNP, an intermediate FRET signal when hybridized to the heterozygous mutant SNP, and a maximal FRET signal when hybridized to the homozygous mutant SNP. These reference values are used in the evaluation of the two signals obtained for each sample DNA. Optimal hybridization conditions and designs of the two nucleic acid probe/accessory molecule complexes can be determined by theoretical design and by experimentation, in order to determine the relative magnitude of the observed signal for an individual DNA sample.

C. Differentially labelled, dual nucleic acid probe/accessory molecule complexes, duplicate or identical DNA samples. This approach is similar to the immediately preceding approach, “Dual nucleic acid probe/accessory molecule complexes, duplicate or identical DNA samples”. Two nucleic acid probes are again used in this approach for a differential assay, one probe being specific for the wild type SNP and the other probe being specific for the mutant SNP. The nature or the location or both of the first and second reporter moieties that form the members of the FRET pair are different in the nucleic acid probe specific for the wild type SNP than in the nucleic acid probe specific for the mutant SNP. Preferably, under the same hybridization conditions, the two probes produce identifiably different signals upon hybridization to their respective targets. For example, the FRET pair for the nucleic acid probe specific for the wild type SNP may have a different donor emission maximum wavelength than that of the FRET pair for the nucleic acid probe specific for the mutant SNP. The two probes are complexed with the accessory molecule, separately or together, and immobilized on the solid substrate. Thus, the two probes can be immobilized on the substrate as individual nucleic acid probe/accessory molecule complexes (in separate spots, or in the same spot containing both nucleic acid probe/accessory molecule complexes), or in a combination complex (a single accessory molecule complexed with both nucleic acid probes). Preferably, the two nucleic acid probe/accessory molecule complexes are immobilized on the same solid substrate (for example, as adjacent but separate spots on a slide or chip, or in a single spot containing both nucleic acid probe/accessory molecule complexes, or in a single spot containing a combination complex), allowing the two probes to be exposed to the DNA sample under essentially identical conditions. The immobilized probe complex or complexes are contacted, separately or simultaneously, with duplicate DNA samples (for example, separate spots, each containing one of the two immobilized probe complexes, are contacted with duplicate aliquots of a solution containing the DNA sample), or with an identical DNA sample (for example, the entire chip or slide, bearing spots of each immobilized probe complex or complexes, is exposed to a single aliquot of a solution containing the DNA sample). The observed detectable signal from each of the two differentially labelled nucleic acid probe/accessory molecule complexes under appropriate hybridization conditions is compared to known values of the signal observed from the two types of nucleic acid probe/accessory molecule complexes hybridized individually to reference samples of the homozygous wild type, heterozygous mutant, and homozygous mutant SNP. The nucleic acid probe/accessory molecule complex specific for the wild type SNP gives a maximal FRET signal when hybridized to the homozygous wild type SNP, an intermediate FRET signal when hybridized to the heterozygous mutant SNP, and a minimal FRET signal when hybridized to the homozygous mutant SNP. The nucleic acid probe/accessory molecule complex specific for the mutant SNP gives a minimal FRET signal when hybridized to the homozygous wild type SNP, an intermediate FRET signal when hybridized to the heterozygous mutant SNP, and a maximal FRET signal when hybridized to the homozygous mutant SNP. These reference values are used in the evaluation of the two differential signals obtained for each sample DNA. Optimal hybridization conditions and designs of the two nucleic acid probe/accessory molecule complexes can be determined by theoretical design and by experimentation, in order to determine the relative magnitude of the observed signal for an individual DNA sample.

All publications, including patent documents and scientific articles, referred to in this application and the bibliography and attachments are incorporated by reference in their entirety for all purposes to the same extent as if each individual publication were individually incorporated by reference.

All headings are for the convenience of the reader and should not be used to limit the meaning of the text that follows the heading, unless so specified.

What is claimed is:

1. A method for detecting a single nucleotide polymorphism in a sample, comprising:
a) providing at least one sample suspected of containing a single nucleotide polymorphism;
b) providing at least one nucleic acid probe, said at least one nucleic acid probe comprising:
(i) a first recognition sequence that is complementary to a first site of a target allelic variant of said single nucleotide polymorphism, wherein said first site of a target allelic variant of said single nucleotide polymorphism comprises a nucleotide at the polymorphic locus of said single nucleotide polymorphism;
(ii) a second recognition sequence that is complementary to a second site of said target allelic variant of said single nucleotide polymorphism;
(iii) a linking element that links said first and second recognition sequences, that is not complementary to either said recognition sequence; and
(iv) a first reporter moiety, located on said first recognition sequence, and a second reporter moiety, wherein
said first reporter moiety and said second reporter moiety are capable of interacting to produce a detectable signal; and
a change in the spatial arrangement of said first reporter moiety relative to said second reporter moiety results in a change in said detectable signal;
c) contacting said at least one sample with said at least one nucleic acid probe;
d) incubating said at least one sample under hybridizing conditions with said at least one nucleic acid probe for a period of time sufficient to permit hybridization between said at least one nucleic acid probe and said target allelic variant of said single nucleotide polymorphism present in said at least one sample, wherein
said hybridization changes said spatial arrangement of said first reporter moiety relative to said second reporter moiety; and
relative said change in said spatial arrangement of said first reporter moiety relative to said second reporter moiety is different when there is a single base-pairing mismatch between said at least one nucleic acid probe and said target allelic variant of said single nucleotide polymorphism present in said at least one sample than when there is no single base-pairing mismatch; and
e) detecting said change in said detectable signal, wherein relative said change in said detectable signal under said hybridization conditions is an indicator of the presence or absence of a single base mismatch between said at least one nucleic acid probe and said target allelic variant of said single nucleotide polymorphism present in said at least one sample.

2. The method of claim 1, wherein said first recognition sequence comprises between about 4 and about 30 bases.

3. The method of claim 1, wherein said first recognition sequence comprises between about 4 and about 15 bases.

4. The method of claim 1, wherein said second recognition sequence comprises between about 4 and about 150 bases.

5. The method of claim 1, wherein said linking element comprises from between about 4 bases to about 300 bases.

6. The method of claim 1, wherein said second reporter moiety is located on said second recognition sequence.

7. The method of claim 1, wherein said second reporter moiety is located on said linking element.

8. The method of claim 6, wherein the location of said first reporter moiety is within about 15 bases from a first terminus of said first recognition sequence of said at least one nucleic acid probe.

9. The method of claim 6, wherein the location of said second reporter moiety is within about 75 bases from a second terminus of said second recognition sequence of said at least one nucleic acid probe.

10. The method of claim 7, wherein the location of said first reporter moiety is within about 15 bases from a first terminus of said first recognition sequence of said at least one nucleic acid probe.

11. The method of claim 1, wherein said detectable signal comprises resonance energy transfer selected from the group consisting of fluorescence resonance energy transfer, luminescence resonance energy transfer, and phosphorescence resonance energy transfer.

12. The method of claim 1, wherein said detectable signal comprises a signal selected from the group consisting of a nuclear magnetic resonance signal, an electron spin resonance signal, an electron paramagnetic resonance signal, an electromagnetic radiation signal, or a change in the physical dimensions of the nucleic acid probe structure.

13. The method of claim 1, wherein said detectable signal comprises an enzymatic reaction.

14. The method of claim 1, wherein said at least one nucleic acid probe comprises a deoxyribonucleic acid, a ribonucleic acid, a nucleic acid mimic, a peptide nucleic acid, a polypeptide, a polymer, or a combination thereof.

15. A method for detecting a single nucleotide polymorphism in a sample, comprising:
a) providing at least one sample suspected of containing a single nucleotide polymorphism;
b) providing at least one nucleic acid probe, said at least one nucleic acid probe comprising:
(i) a first recognition sequence that is complementary to a first site of a target allelic variant of said single nucleotide polymorphism, wherein said first site of a target allelic variant of said single nucleotide polymorphism comprises a nucleotide at the polymorphic locus of said single nucleotide polymorphism;
(ii) a second recognition sequence that is complementary to a second site of said target allelic variant of said single nucleotide polymorphism;
(iii) a linking element that links said first and second recognition sequences, that is not complementary to either said recognition sequence; and
(iv) a first reporter moiety, located on said first recognition sequence, and a second reporter moiety, wherein
said first reporter moiety and said second reporter moiety are capable of interacting to produce a detectable signal; and
a change in the spatial arrangement of said first reporter moiety relative to said second reporter moiety results in a change in said detectable signal;
c) providing at least one accessory molecule;
d) contacting said at least one nucleic acid probe with said at least one accessory molecule;
e) contacting said at least one nucleic acid probe and said at least one accessory molecule with said at least one sample;
f) incubating said at least one sample under hybridizing conditions with said at least one nucleic acid probe and said at least one accessory molecule for a period of time sufficient to permit hybridization between said at least one nucleic acid probe and said target allelic variant of said single nucleotide polymorphism present in said at least one sample, wherein
said hybridization changes said spatial arrangement of said first reporter moiety relative to said second reporter moiety; and
relative said change in said spatial arrangement of said first reporter moiety relative to said second reporter moiety is different when there is a single base-pairing mismatch between said at least one nucleic acid probe and said target allelic variant of said single nucleotide polymorphism present in said at least one sample than when there is no single base-pairing mismatch; and
g) detecting said change in said detectable signal, wherein relative said change in said detectable signal under said hybridization conditions is an indicator of the presence or absence of a single base-pairing mismatch between said at least one nucleic acid probe and said target allelic variant of said single nucleotide polymorphism present in said at least one sample.

16. The method of claim 15, wherein said at least one accessory molecule comprises a deoxyribonucleic acid, a ribonucleic acid, a nucleic acid mimic, a peptide nucleic acid, a polypeptide, a polymer, or a combination thereof.

17. The method of claim 15, wherein said first recognition sequence comprises between about 4 and about 30 bases.

18. The method of claim 15, wherein said first recognition sequence comprises between about 4 and about 15 bases.

19. The method of claim 15, wherein said second recognition sequence comprises between about 4 and about 150 bases.

20. The method of claim 15, wherein said linking element comprises from between about 4 bases to about 300 bases.

21. The method of claim 15, wherein said second reporter moiety is located on said second recognition sequence.

22. The method of claim 15, wherein said second reporter moiety is located on said linking element.

23. The method of claim 21, wherein the location of said first reporter moiety is within about 15 bases from a first terminus of said first recognition sequence of said at least one nucleic acid probe.

24. The method of claim 21, wherein the location of said second reporter moiety is within about 75 bases from a second terminus of said second recognition sequence of said at least one nucleic acid probe.

25. The method of claim 22, wherein the location of said first reporter moiety is within about 15 bases from a first terminus of said first recognition sequence of said at least one nucleic acid probe.

26. The method of claim 15, wherein said detectable signal comprises energy transfer selected from the group consisting of fluorescence resonance energy transfer, luminescence resonance energy transfer, and phosphorescence resonance energy transfer.

27. The method of claim 15, wherein said detectable signal is a signal selected from the group consisting a nuclear magnetic resonance signal, an electron spin resonance signal, an electron paramagnetic resonance signal, and an electromagnetic radiation signal, or a change in the physical dimensions of the nucleic acid probe structure.

28. The method of claim 15, wherein said detectable signal comprises an enzymatic reaction.

29. The method of claim 15, wherein said at least one nucleic acid probe comprises a deoxyribonucleic acid, a ribonucleic acid, a nucleic acid mimic, a peptide nucleic acid, a polypeptide, a polymer, or a combination thereof.

30. The method of claim 15, wherein said at least one accessory molecule helps to maintain a spatial arrangement between said first reporter moiety and said second reporter moiety that is different when said at least one nucleic acid probe is hybridized to said target allelic variant of said single nucleotide polymorphism present in said at least one sample than when not hybridized.

31. The method of claim 15, wherein said at least one accessory molecule enhances the hybridization between said at least one nucleic acid probe and said target allelic variant of said single nucleotide polymorphism present in said at least one sample.

32. The method of claim 15, wherein said at least one accessory molecule serves to tether said at least one nucleic acid probe to a solid surface.

33. A method for detecting a single nucleotide polymorphism in a sample, comprising:
a) providing at least one sample suspected of containing a single nucleotide polymorphism;
b) providing at least one nucleic acid probe, said at least one nucleic acid probe comprising:
(i) a first recognition sequence that is complementary to a first site of a target allelic variant of said single nucleotide polymorphism, wherein said first site of a target allelic variant of said single nucleotide polymorphism comprises a nucleotide at the polymorphic locus of said single nucleotide polymorphism;
(ii) a second recognition sequence that is complementary to a second site of said target allelic variant of said single nucleotide polymorphism;
(iii) a linking element that links said first and second recognition sequences, that is not complementary to either said recognition sequence; and
(iv) a first reporter moiety, located on said first recognition sequence;
c) providing at least one accessory molecule, said at least one accessory molecule comprising a second reporter moiety, wherein
said first reporter moiety and said second reporter moiety are capable of interacting to produce a detectable signal; and
a change in the spatial arrangement of said first reporter moiety relative to said second reporter moiety results in a change in said detectable signal;
d) contacting said at least one nucleic acid probe with said at least one accessory molecule;
e) contacting said at least one nucleic acid probe and said at least one accessory molecule with said at least one sample;
f) incubating said at least one sample under hybridizing conditions with said at least one nucleic acid probe and said at least one accessory molecule for a period of time sufficient to permit hybridization between said at least one nucleic acid probe and said target allelic variant of said single nucleotide polymorphism present in said at least one sample, wherein
said hybridization changes said spatial arrangement of said first reporter moiety relative to said second reporter moiety; and
relative said change in said spatial arrangement of said first reporter moiety relative to said second reporter moiety is different when there is a single base-pairing mismatch between said at least one nucleic acid probe and said target allelic variant of said single nucleotide polymorphism present in said at least one sample than when there is no single base-pairing mismatch; and
g) detecting said change in said detectable signal, wherein relative said change in said detectable signal under said hybridization conditions is an indicator of the presence or absence of a single base-pairing mismatch between said at least one nucleic acid probe and said target allelic variant of said single nucleotide polymorphism present in said at least one sample.

34. The method of claim 33, wherein said at least one accessory molecule comprises a deoxyribonucleic acid, a ribonucleic acid, a nucleic acid mimic, a peptide nucleic acid, a polypeptide, a polymer, or a combination thereof.

35. The method of claim 33, wherein said first recognition sequence comprises between about 4 and about 30 bases.

36. The method of claim 33, wherein said first recognition sequence comprises between about 4 and about 15 bases.

37. The method of claim 33, wherein said second recognition sequence comprises between about 4 and about 150 bases.

38. The method of claim 33, wherein said linking element comprises from between about 4 bases to about 300 bases.

39. The method of claim 33, wherein the location of said first reporter moiety is within about 15 bases from a terminus of said first recognition sequence of said at least one nucleic acid probe.

40. The method of claim 33, wherein said detectable signal comprises energy transfer selected from the group consisting of fluorescence resonance energy transfer, luminescence resonance energy transfer, and phosphorescence resonance energy transfer.

41. The method of claim 33, wherein said detectable signal comprises a signal selected from the group consisting of a nuclear magnetic resonance signal, an electron spin resonance signal, an electron paramagnetic resonance signal, and an electromagnetic radiation signal, or a change in the physical dimensions of the nucleic acid probe structure.

42. The method of claim 33, wherein said detectable signal comprises an enzymatic reaction.

43. The method of claim 3, wherein said at least one nucleic acid probe comprises a deoxyribonucleic acid, a ribonucleic acid, a nucleic acid mimic, a peptide nucleic acid, a polypeptide, a polymer, or a combination thereof.

44. The method of claim 33, wherein said at least one accessory molecule helps to maintain a spatial arrangement between said first reporter moiety and said second reporter moiety that is different when said at least one nucleic acid probe is hybridized to said target allelic variant of said single nucleotide polymorphism present in said at least one sample than when not hybridized.

45. The method of claim 33, wherein said at least one accessory molecule enhances the hybridization between said at least one nucleic acid probe and said target allelic variant of said single nucleotide polymorphism present in said at least one sample.

46. The method of claim 33, wherein said at least one accessory molecule serves to tether said at least one nucleic acid probe to a solid surface.

47. A nucleic acid probe for detecting a single nucleotide polymorphism in a nucleic acid sample sequence, comprising:
(a) a first recognition sequence that is complementary to a first site of a target allelic variant of said single nucleotide polymorphism, wherein said first site of a target allelic variant of said single nucleotide polymorphism comprises a nucleotide at the polymorphic locus of said single nucleotide polymorphism;
(b) a second recognition sequence that is complementary to a second site of said target allelic variant of said single nucleotide polymorphism;
(c) a linking element that links said first and second recognition sequences, that is not complementary to either said recognition sequence; and
(d) a first reporter moiety, located on said first recognition sequence, and a second reporter moiety, wherein
said first reporter moiety and said second reporter moiety are capable of interacting to produce a detectable signal; and
a change in the spatial arrangement of said first reporter moiety relative to said second reporter moiety results in a change in said detectable signal.

48. A nucleic acid probe for detecting a single nucleotide polymorphism in a nucleic acid sample sequence, comprising:
(a) a first recognition sequence that is complementary to a first site of a target allelic variant of said single nucleotide polymorphism, wherein said first site of a target allelic variant of said single nucleotide polymorphism comprises a nucleotide at the polymorphic locus of said single nucleotide polymorphism;
(b) a second recognition sequence that is complementary to a second site of said target allelic variant of said single nucleotide polymorphism;
(c) a linking element that links said first and second recognition sequences, that is not complementary to either said recognition sequence; and
(d) a first reporter moiety, located on said first recognition sequence, wherein
said first reporter moiety and a second reporter moiety that is located on an accessory molecule are capable of interacting to produce a detectable signal; and
a change in the spatial arrangement of said first reporter moiety relative to said second reporter moiety results in a change in said detectable signal.


SEQUENCE LISTING (26 sequences)

SEQ ID NO: 1 - DNA, 42 bp, artificial sequence
  synthetic construct
tgtagtatcgtggctgtgtaatcatagcggcaccaactggca

SEQ ID NO: 2 - DNA, 48 bp, artificial sequence
  synthetic construct
ctgacgctggttgcatcggacgatactacatgccagttggactaacgg

SEQ ID NO: 3 - DNA, 47 bp, artificial sequence
  synthetic construct
gatggcgacatcctgccgctatgattacacagcctgagcattgacac

SEQ ID NO: 4 - DNA, 11 bp, artificial sequence
  synthetic construct
tgtagtatcgt

SEQ ID NO: 5 - DNA, 10 bp, artificial sequence
  synthetic construct
ccaactggca

SEQ ID NO: 6 - DNA, 21 bp, artificial sequence
  synthetic construct
ggctgtgtaatcatagcggca

SEQ ID NO: 7 - DNA, 11 bp, artificial sequence
  synthetic construct
acgatactaca

SEQ ID NO: 8 - DNA, 10 bp, artificial sequence
  synthetic construct
tgccagttgg

SEQ ID NO: 9 - DNA, 21 bp, artificial sequence
  synthetic construct
tgccgctatgattacacagcc

SEQ ID NO: 10 - DNA, 31 bp, artificial sequence
  synthetic construct
atcggacgatactacatgccagttggactaa

SEQ ID NO: 11 - DNA, 31 bp, artificial sequence
  synthetic construct
atcggacgctactacatgccagttggactaa

SEQ ID NO: 12 - DNA, 31 bp, artificial sequence
  synthetic construct
atcggacgacactacatgccagttggactaa

SEQ ID NO: 13 - DNA, 31 bp, artificial sequence
  synthetic construct
atcggacgatcctacatgccagttggactaa

SEQ ID NO: 14 - DNA, 11 bp, artificial sequence
  synthetic construct
acgctactaca

SEQ ID NO: 15 - DNA, 11 bp, artificial sequence
  synthetic construct
acgacactaca

SEQ ID NO: 16 - DNA, 11 bp, artificial sequence
  synthetic construct
acgatcctaca

SEQ ID NO: 17 - DNA, 95 bp, artificial sequence
  synthetic construct
gctgctgtccgatgcggtcactggttagtccatgatgcacggtagcgccgttagtccaactggcatgtagtatcgtccgatgcaaccagcgtcag

SEQ ID NO: 18 - DNA, 24 bp, artificial sequence
  synthetic construct
tcggacagcagcctgacgctggtt

SEQ ID NO: 19 - DNA, 95 bp, artificial sequence
  synthetic construct
tcggacagcagcctgacgctggttgcatcggacgatactacatgccagttggactaacggcgctaccgtgcatcatggactaaccagtgaccgca

SEQ ID NO: 20 - DNA, 42 bp, artificial sequence
  synthetic construct
cctggcacgtaggctgtgtaatcatagcggcagggtgctcca

SEQ ID NO: 21 - DNA, 11 bp, artificial sequence
  synthetic construct
cctggcacgta

SEQ ID NO: 22 - DNA, 10 bp, artificial sequence
  synthetic construct
gggtgctcca

SEQ ID NO: 23 - DNA, 48 bp, artificial sequence
  synthetic construct
gaagagcagagatatacgtgccaggtggagcacccaggcctggatcag

SEQ ID NO: 24 - DNA, 48 bp, artificial sequence
  synthetic construct
gaagagcagagatatacgtaccaggtggagcacccaggcctggatcag

SEQ ID NO: 25 - DNA, 11 bp, artificial sequence
  synthetic construct
tacgtgccagg

SEQ ID NO: 26 - DNA, 10 bp, artificial sequence
  synthetic construct
tggagcaccc